(19) United States
(12) Patent Application Publication
(10) Pub. No.: US 2024/0182904 A1
(43) Pub. Date: Jun. 6, 2024

(54) OLIGONUCLEOTIDES TARGETING S6K1

(71) Applicant: UNIVERSITY OF MASSACHUSETTS, Boston, MA (US)

(72) Inventors: Claudio Punzo, Brookline, MA (US); Anastasia Khvorova, Westborough, MA (US); Dimas Echeverria Moreno, Worcester, MA (US); Annabelle Biscans, Gothenburg (SE); Julia F. Alterman, Westborough, MA (US); Matthew Hassler, Boston, MA (US); Shun-Yun Cheng, Westborough, MA (US); Jillian Caiazzi, Boston, MA (US)

(21) Appl. No.: 18/375,235

(22) Filed: Sep. 29, 2023

Related U.S. Application Data
(60) Provisional application No. 63/412,092, filed on Sep. 30, 2022.

(51) Int. Cl. C12N 15/113; A61K 47/54; A61P 27/02; C12N 15/86

(52) U.S. Cl. CPC C12N 15/1137; A61K 47/549; A61P 27/02; C12N 15/86; C12N 2310/14; C12N 2310/315; C12N 2310/321; C12N 2310/3515; C12N 2750/14122; C12N 2750/14143

(57) ABSTRACT

Provided herein are oligonucleotides (e.g., siRNA) targeting S6K1. Also provided are methods of treating a disease associated with S6K1 expression.

### RELATED APPLICATIONS

The present invention claims the benefit of U.S. Provisional Patent Application Ser. No. 63/412,092, filed Sep. 30, 2022, the content of which is incorporated herein by reference in its entirety for all purposes.

### SEQUENCE LISTING

The instant application contains a Sequence Listing which has been submitted electronically in XML format and is hereby incorporated by reference in its entirety. Said XML file, created on Jan. 23, 2024, is named 744225_UM9-291_ST26.xml and is 147,834 bytes in size.

### BACKGROUND

Ribosomal protein S6 kinase B1 (RPS6KB1), also known as S6K1, is a serine/threonine kinase that phosphorylates the S6 ribosomal protein to stimulate protein synthesis. S6K1 has been implicated in several disease-states, including cancer, obesity, diabetes and insulin resistance, and macular degeneration (such as age-related macular degeneration (AMD)).

Age-related macular degeneration is the leading cause of blindness in the elderly of the industrialized world. Disease generally initiates with the formation of “Drusen,” which are lipoprotein-rich deposits that form between the Bruch's membrane (BrM) and the retinal-pigmented epithelium (RPE) or between the RPE and the photoreceptor (PR) outer segments. Twenty percent of individual with drusen progress to the advanced forms of the disease, which is characterized by geographic atrophy (GA) of the RPE and the underlying PRs or by neovascular pathologies. The only treatment available to date is in regard to the neovascular pathology (also referred to as “wet AMD”), which uses anti-angiogenesis antibodies to inhibit the action of the “vascular endothelial growth factor” (VEGF). There is no treatment to prevent progression from the early disease stages to the advanced stages. Nor is there a treatment available for the advanced form of GA (often referred to as “dry” AMD).

Oligonucleotides such as small interfering RNA (siRNA) molecules have been used to regulate gene expression levels across different organs. Their implementation in the eye, however, has been hampered by low permeability of the siRNA molecule into various cell types, stability of the siRNA and longevity of the knockdown effect. Described herein are oligonucleotides that are effective at silencing S6K1 expression.

### SUMMARY

In one aspect, the disclosure provides an siRNA comprising a sense strand and an antisense strand, wherein the antisense strand comprises a sequence substantially complementary to a S6K1 nucleic acid sequence of any one of SEQ ID NOs: 1-6 (i.e., the antisense strand comprises a sequence substantially complementary to a S6K1 nucleic acid sequence of SEQ ID NO: 1, SEQ ID NO: 2, SEQ ID NO: 3, SEQ ID NO: 4, SEQ ID NO: 5, or SEQ ID NO: 6).

In certain embodiments, the antisense strand comprises a sequence substantially complementary to a S6K1 nucleic acid sequence of any one of SEQ ID NOs: 7-12. In certain embodiments, the antisense strand comprises a sequence substantially complementary to a S6K1 nucleic acid sequence of SEQ ID NO: 7. In certain embodiments, the antisense strand comprises a sequence substantially complementary to a S6K1 nucleic acid sequence of SEQ ID NO: 8. In certain embodiments, the antisense strand comprises a sequence substantially complementary to a S6K1 nucleic acid sequence of SEQ ID NO: 9. In certain embodiments, the antisense strand comprises a sequence substantially complementary to a S6K1 nucleic acid sequence of SEQ ID NO: 10. In certain embodiments, the antisense strand comprises a sequence substantially complementary to a S6K1 nucleic acid sequence of SEQ ID NO: 11. In certain embodiments, the antisense strand comprises a sequence substantially complementary to a S6K1 nucleic acid sequence of SEQ ID NO: 12.

In certain embodiments, the siRNA comprises complementarity to at least 10, 11, 12 or 13 contiguous nucleotides of the S6K1 nucleic acid sequence of any one of SEQ ID NOs: 1-6.

In certain embodiments, the siRNA comprises no more than 3 mismatches with the S6K1 nucleic acid sequence of any one of SEQ ID NOs: 1-6.

In certain embodiments, the siRNA comprises full complementarity to the S6K1 nucleic acid sequence of any one of SEQ ID NOs: 1-6.

In certain embodiments, the antisense strand comprises about 15 nucleotides to 25 nucleotides in length. In certain embodiments, the sense strand comprises about 15 nucleotides to 25 nucleotides in length. In certain embodiments, the antisense strand is 20 nucleotides in length. In certain embodiments, the antisense strand is 21 nucleotides in length. In certain embodiments, the antisense strand is 22 nucleotides in length. In certain embodiments, the sense strand is 15 nucleotides in length. In certain embodiments, the sense strand is 16 nucleotides in length. In certain embodiments, the sense strand is 18 nucleotides in length. In certain embodiments, the sense strand is 20 nucleotides in length.

In certain embodiments, the siRNA of comprises a double-stranded region of 15 base pairs to 20 base pairs. In certain embodiments, the siRNA of comprises a double-stranded region of 15 base pairs. In certain embodiments, the siRNA of comprises a double-stranded region of 16 base pairs. In certain embodiments, the siRNA of comprises a double-stranded region of 18 base pairs. In certain embodiments, the siRNA of comprises a double-stranded region of 20 base pairs.

In certain embodiments, the siRNA comprises at least one blunt-end.

In certain embodiments, the siRNA comprises at least one single stranded nucleotide overhang. In certain embodiments, the siRNA comprises about a 2-nucleotide to 5-nucleotide single stranded nucleotide overhang. In certain embodiments, the siRNA comprises 2-nucleotide single stranded nucleotide overhang. In certain embodiments, the siRNA comprises 5-nucleotide single stranded nucleotide overhang. In certain embodiments, the siRNA comprises naturally occurring nucleotides.

In certain embodiments, the siRNA comprises at least one modified nucleotide. In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified intemucleotide linkage. In certain embodiments, the modified intemucleotide linkage comprises a phosphorothioate intemucleotide linkage. In certain embodiments, the siRNA comprises 4-16 phosphorothioate intemucleotide linkages. In certain embodiments, the siRNA comprises 8-13 phosphorothioate intemucleotide linkages. In certain embodiments, the antisense strand comprises 2-10 phosphorothioate intemucleotide linkages.

In certain embodiments, the siRNA comprises at least 80% chemically modified nucleotides. In certain embodiments, the siRNA is fully chemically modified.

In certain embodiments, the siRNA comprises at least 70% 2′-O-methyl nucleotide modifications. In certain embodiments, the antisense strand comprises at least 70% 2′-O-methyl nucleotide modifications.

In certain embodiments, the antisense strand comprises about 70% to 90% 2′-O-methyl nucleotide modifications. In certain embodiments, the sense strand comprises at least 65% 2′-O-methyl nucleotide modifications. In certain embodiments, the sense strand comprises 100% 2′-O-methyl nucleotide modifications.

In certain embodiments, the sense strand comprises one or more nucleotide mismatches between the antisense strand and the sense strand. In certain embodiments, the one or more nucleotide mismatches are present at positions 2, 6, and 12 from the 5′ end of sense strand. In certain embodiments, the nucleotide mismatches are present at positions 2, 6, and 12 from the 5′ end of the sense strand.

In certain embodiments, the antisense strand comprises a 5′ phosphate, a 5′-alkyl phosphonate, a 5′ alkylene phosphonate, or a 5′ alkenyl phosphonate.

In certain embodiments, the antisense strand comprises a 5′ vinyl phosphonate. In certain embodiments, a functional moiety is linked to the 5′ end and/or 3′ end of the antisense strand. In certain embodiments, a functional moiety is linked to the 5′ end and/or 3′ end of the sense strand. In certain embodiments, a functional moiety is linked to the 3′ end of the sense strand.

In certain embodiments, the functional moiety comprises a hydrophobic moiety.

In certain embodiments, the hydrophobic moiety is selected from the group consisting of fatty acids, steroids, secosteroids, lipids, gangliosides, nucleoside analogs, endocannabinoids, vitamins, and a mixture thereof.

In certain embodiments, the steroid selected from the group consisting of cholesterol and lithocholic acid (LA).

In certain embodiments, the fatty acid selected from the group consisting of Eicosapentaenoic acid (EPA), Docosahexaenoic acid (DHA) and Docosanoic acid (DCA).

In certain embodiments, the vitamin is selected from the group consisting of choline, vitamin A, vitamin E, and derivatives or metabolites thereof.

In certain embodiments, the vitamin is selected from the group consisting of retinoic acid and alphα-tocopheryl succinate.

In certain embodiments, the functional moiety comprises any one of triple amine, retinoic acid, docosahexaenoic acid (DHA), docosanoic acid (DCA), α-tocopheryl succinate, or lithocholic acid.

In certain embodiments, the lithocholic acid is a natural lithocholic acid.

In certain embodiments, the lithocholic acid is an isomeric lithocholic acid.

In certain embodiments, the functional moiety is linked to the antisense strand and/or sense strand by a linker.

In certain embodiments, the linker comprises a divalent or trivalent linker.

In certain embodiments, the divalent or trivalent linker is selected from the group consisting of:

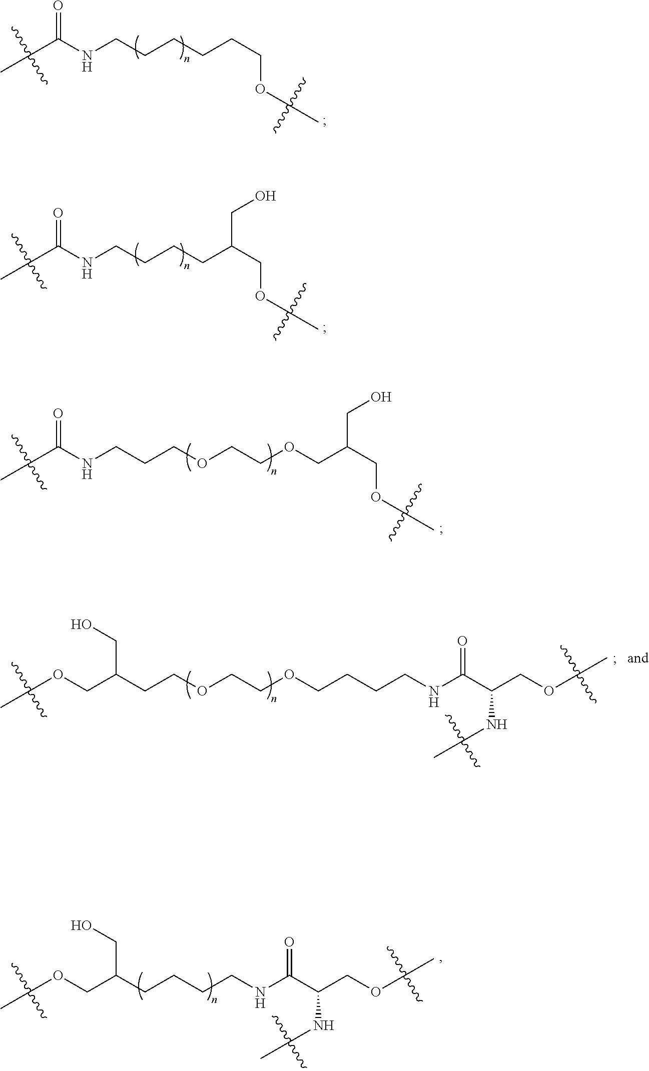

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, when the linker is a trivalent linker, the linker further links a phosphodiester or phosphodiester derivative.

In certain embodiments, the phosphodiester or phosphodiester derivative is selected from the group consisting of:

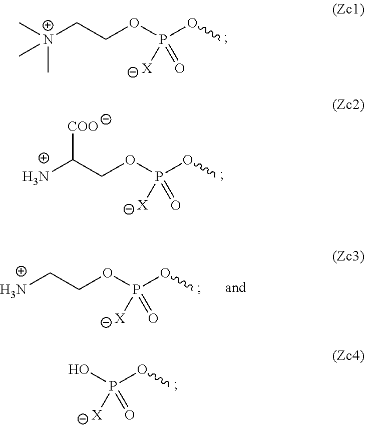

wherein X is O, S or BH₃.

In certain embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In one aspect, the disclosure provides a pharmaceutical composition for inhibiting the expression of S6K1 gene in an organism, comprising the siRNA described above and a pharmaceutically acceptable carrier.

In certain embodiments, the siRNA inhibits the expression of said S6K1 gene by at least 20%. In certain embodiments, the siRNA inhibits the expression of said S6K1 gene by at least 50%.

In one aspect, the disclosure provides a method for inhibiting expression of S6K1 gene in a cell, the method comprising: (a) introducing into the cell the siRNA described above; and (b) maintaining the cell produced in step (a) for a time sufficient to obtain degradation of the mRNA transcript of the S6K1 gene, thereby inhibiting expression of the S6K1 gene in the cell.

In one aspect, the disclosure provides a method of treating or managing a eye disorder comprising administering to a patient in need of such treatment a therapeutically effective amount of the siRNA described above.

In certain embodiments, the siRNA is administered to the eye of the patient.

In certain embodiments, the siRNA is administered by intravitreal injection.

In certain embodiments, the siRNA inhibits the expression of said S6K1 gene by at least 20%. In certain embodiments, the dsRNA inhibits the expression of said S6K1 gene by at least 50%.

In one aspect, the disclosure provides a vector comprising a regulatory sequence operably linked to a nucleotide sequence that encodes a dsRNA substantially complementary to a S6K1 nucleic acid sequence of SEQ ID NOs: 1-6.

In certain embodiments, the dsRNA inhibits the expression of said S6K1 gene by at least 20%. In certain embodiments, the dsRNA inhibits the expression of said S6K1 gene by at least 50%.

In certain embodiments, the dsRNA comprises a sense strand and an antisense strand, wherein the antisense strand comprises a sequence substantially complementary to a S6K1 nucleic acid sequence of SEQ ID NOs: 1-6.

In one aspect, the disclosure provides a cell comprising the vector described above.

In one aspect, the disclosure provides a recombinant adeno-associated virus (rAAV) comprising the vector described above and an AAV capsid.

In one aspect, the disclosure provides a branched RNA compound comprising two or more of the siRNA described above covalently bound to one another.

In certain embodiments, siRNA are covalently bound to one another by way of a linker, spacer, or branching point.

### BRIEF DESCRIPTION OF THE DRAWINGS

FIG. 1 shows retinal cross sections with 12 different siRNA distributions 3 days after injection of 0.3 nanomoles of siRNA (left panels: retinoic acid (RA), Docosahexaenoic acid (DHA), phosphocholine (PC); α-tocopheryl succinate (TS); docosanoic acid (DCA)). To the right: One example per group showing entire retinal cross section with distribution across the entire retina. All siRNAs are labeled with Cy3 and are shown in red. Glutamine synthetase (GS) expression, is shown in green. Nuclear DAPI is shown in blue. To the right: One example per group showing entire retinal cross section with distribution across the entire retina.

FIG. 2 shows the enrichment of siRNA in different retinal cell types arranged by cell type. The bars show relative protein levels of cell type specific markers calibrated to total retinal extracts of an un-injected mouse retina.

FIG. 3 shows the enrichment of siRNA in different retinal cell types arranged by modifications. The bars show relative protein levels of cell type specific markers calibrated to total retinal extracts of an un-injected mouse retina. For each modification (i.e., monomer, dimer, trimer, etc.) each bar, from left to right, represents rhodopsin (rods), cone arrestin (CA)(cones), glutamine synthetase (GS) (muller cells), Vglut2 (ganglion cells), VGAT (amacrine cells), protein kinase C alpha (PKCa) (bipolar cells), and Lim1 (horizontal cells).

FIG. 4 shows examples of siRNA distributions in retinal cross sections 3 days after injection of 0.3 nanomole of siRNA. siRNA is labeled shows with Cy3 and is shown in red. All siRNAs are targeting the Huntington gene. Shown is the non-targeting control (NTC) with the PC-TS modification, the trimer, and the tetramer. Cone segments are highlighted in green labeled by PNA (peanut agglutinin lectin), Müller glia cells are shown in cyan, labeled by Glutamine synthetase (GS) and nuclei are shown in blue labeled by DAPI.

FIG. 5 shows examples of siRNA distributions in retinal cross sections 3 days after injection of 0.3 nanomole of siRNA without GS staining in cyan. Additionally, for the trimer and tetramer only the higher magnification of the outer nuclear layer (ONL) is shown to highlight the distribution of the siRNA in the photoreceptor layer. On half of the panel only the siRNA is shown to better visualize signal.

FIG. 6 shows antibody staining on retinal cross sections for HTT protein two weeks after injection with the Htt-siRNA. First column shows staining in control mice injected with the NTC-siRNA. Second column, expression of HTT protein after knockdown with PC-RA-Htt siRNA. Shown are examples of 2 different mice each injected with ˜0.3 nanomoles of the Htt-siRNA.

FIG. 7 shows the quantification by western blotting of total HTT protein two weeks after injection with the Htt-siRNA. Same experimental setting as in FIG. 6 (different mice of the same injected batch) quantifying total HTT protein remaining from total retinal extracts.

FIG. 8 shows the quantification by bDNA assay to quantify total Htt mRNA levels two weeks after injection with the Htt-siRNA using 0.1 nanomole per injection of stated siRNA modification.

FIG. 9 shows quantification by bDNA assay to quantify total Htt mRNA levels 3 days after injection with the Htt-siRNA using 0.3 nanomoles per injection of stated siRNA modification. Each dot represents 1 retina.

FIG. 10 shows the quantification by bDNA assay to quantify total Htt mRNA levels 100 days after injection with the Htt-siRNA using 0.3 nanomoles per injection of stated siRNA modification. Each dot represents 1 retina.

FIG. 11 shows representative fundus images over time of eyes injected with the Cy3 labeled siRNAs with modifications as indicated. Exposure of fluorescence signal is the same for all 4 siRNA at any given time point, but not over time. This figure complements FIG. 10 showing the fundus images of the mice used in FIG. 10. All mice were injected with 0.3 nanomoles of siRNA intravitreally.

FIG. 12 shows dose escalation study of for HTT-knockdown in retina. Mice were injected with amounts indicated in FIG. 1-60 microgram [note: not nanomoles] of Cy3 labeled Tetramer with Htt-siRNA) in a total volume of 2 microliter. Five mice were injected per amount of siRNA. Tissue was harvested at 2 weeks post-injection to perform quantification by western blotting of remaining HTT protein in retina. Injections with 15-30 microgram correspond roughly to the same knockdown seen with ˜0.3 nanomoles in previous experiments.

FIG. 13 shows fundus images of dose escalation study shown in FIG. 12. Images were taken before euthanasia at 2 weeks post-injection. Regular brightfield fundus image as well as Cy3 image is shown for each concentration.

FIG. 14 shows retinal cross sections of eyes from of dose escalation study shown in FIGS. 12 and 13. Images show Cy3 distribution across entire retinal section, indicating that the siRNA is taken up uniformly across the entire eye.

FIG. 15 shows retinal cross sections of eyes from of dose escalation study shown in FIG. 14 stained with Iba1 (green) to identify Iba1 positive cells that migrate to the outer nuclear layer (ONL) where photoreceptors reside. Half of each panel (dotted line) shows only the Iba1 signal to better visualize the signal Blue shows nuclear DAPI.

FIG. 16 shows retinal cross sections of eyes from of dose escalation study shown in FIG. 14 stained with GFAP (red) to identify reactive gliosis in Müller glia cells. siRNA is not shown as these are sections from the same eyes as shown in FIG. 15. Blue shows nuclear DAPI and green marks cone photoreceptor segment with peanut agglutinin lectin (PNA).

FIG. 17 shows measurements of photoreceptor and retinal function by electroretinography under scotopic (0.01 cd.s/m2-1cd.s/m2) and photopic conditions (3 & 10 flashes). A-waves and b-waves are recorded at several amounts injected.

FIG. 18 shows fluorescence intensity of Tetramer-Htt-Cy3 after intravitreal delivery in pig eye. Delivery of amount of siRNA is shown on top of each panel (100-1500 microgram of Tetramer. Top row shows the Cy3 fluorescence of unfixed tissue right after opening the eye. On the bottom of the figure is a higher magnification of a region from the top panel.

FIG. 19 shows the knockdown of Huntington protein in Swine as measured by western blot analysis from eyes shown in FIG. 18. Knockdown was compared to Huntington protein levels in the NTC that was injected with 250 ug of the Tetramer-siRNA-Cy3. Top figure shows knockdown in bar graphs seen in the four major retinal quadrants (DT: Dorsal-Temporal; DN: Dorsal-Nasal; VT: Temporal-Nasal; VN: Ventral-Nasal). Middle panel shows knockdown on a flat mount cartoon with corresponding values of the regional knockdown shown in the bar graph. Bottom panel: Average knockdown of Huntington protein across the entire retina calculated by averaging the knockdown seen in each quadrant per retina. Data shown represents one biological sample for each amount of siRNA delivered. Error bar in first panel is generated by technical replicates. Error bar in last panel is generated by averaging the 4 data points for each quadrant per retina.

FIG. 20 shows antibody staining for Huntington protein on section of eyes injected with different amount as shown in FIG. 19. Area of section is shown in middle panel of FIG. 19.

FIG. 21 shows antibody staining for GFAP (glial fibrillary acidic protein) and Iba1 (ionized calcium binding adaptor protein 1) (as shown in mouse on FIGS. 15 and 16) expression on retinal section of eyes injected with different amount as shown in FIGS. 18 and 19 to determine dose dependent toxicity. GFAP and Iba1 are both shown in green as indicated to the left of each row. Red staining shown siRNA distribution across the retinal section. Nuclei are marked with nuclear DAPI. Half of each panel shows only the signal of interest (siRNA, GFAP or Iba1) to better visualize the signal.

FIG. 22 shows initial knockdown efficiency in vitro of siRNA duplexes formed from the sense and antisense strands shown in Table 3 and 4.

FIG. 23 shows dose response curves for duplexes 2, 3, 9, and 10 from FIG. 22.

FIG. 24 shows an RNA-Scope in situ hybridization on retinal cross-sections of mice to detect the siRNA-tetramer against S6K1. Top row shows sections from 3 mice injected with the NTC for S6K1 in the tetramer configuration. Middle row shows sections from 3 mice injected with the 3 μg/eye with the siRNA against S6K1 in the tetramer configuration. Last row shows sections from 3 mice injected with the 6 μg/eye with the siRNA against S6K1 in the tetramer configuration. The siRNA was delivered intravitreally and animals were euthanized 2 weeks post injection.

FIG. 25A-FIG. 25B show knockdown of S6K1 in mouse after intravitreal injection of 6 μg of siRNA in the tetramer configuration. FIG. 25A shows S6K1 protein levels as detected by western blot 2-weeks post injection. FIG. 25B shows similar data as first graph at 2 months post-injection. Each dot in the graphs represent one biological sample (retina) from one animal.

FIG. 26 shows the knockdown of S6K1 protein in non-human primate (NHP). Western blot data with retinal protein extracts form the superior-temporal (ST) regions (AKA: dorsal-temporal) of one NHP injected intravitreally with 225 μg of S6K1-tetramer (in 75 μL) and 6 naïve NHP retinas from the same region. First set of bar graphs shows a comparison between the uninjected contralateral eye and the S6K1 siRNA injected one to allow for a direct intra-animal comparison between both eyes. The second bar graph shows a comparison between the 6 naïve NHPs and the S6K1 siRNA injected one. NHP eyes were harvested 1-month post-injection. Shown is also the phosphorylation of ribosomal protein S6, which is a canonical target of S6K1. Similar to the S6K1 knockdown data, intra-animal comparison is shown to the left and comparison with several NHPs is shown to the right.

FIG. 27 shows the knockdown of S6K1 protein on retinal cross section of non-human primate (NHP) after siRNA treatment. Data is generated with the one injected eye and the uninjected contralateral eye. Sections were obtained from the central regions as shown for the pig in FIG. 19. To the left: entire cross section encompassing the fovea. To the right: higher magnification of temporal and nasal regions as well as the fovea. Top row shows uninjected eye and bottom row eye injected intravitreally with 225 μg of S6K1-tetramer (in 75 4).

FIG. 28 shows the reduction in phosphorylated S6 protein (pS6) on retinal cross section of non-human primate (NHP) after siRNA treatment. Data is same as shown in FIG. 27, with the exception that the staining probes for the expression of pS6 (red signal). In each panel green and blue signal have been removed from half the panel (dotted line) to better visualize the knockdown of pS6. Blue shows nuclear DAPI and green shows cones segments marked with peanut agglutinin lectin (PNA).

FIG. 29 shows the expression of inflammatory markers in NHP after siRNA treatment with S6K1 siRNA (75 microliter, 225 μg of siRNA in tetramer configuration). Data is same as shown in FIG. 27 and FIG. 28, with the exception that the staining probes for the expression of Iba1 (red signal, first set) and GFAP (red signal, second set). Untreated contralateral eye is in first row of each set and the treated one in the second row. In each panel green and blue signal have been removed from half the panel (dotted line) to better visualize the Iba1 and GFAP signal. Blue shows nuclear DAPI and green shows cones segments marked with peanut agglutinin lectin (PNA).

### DETAILED DESCRIPTION

The present disclosure relates to oligonucleotide conjugates and branched oligonucleotides that are capable of efficient gene knockdown in the eye. Several different functional moieties and branched oligonucleotides demonstrated eye cell specific delivery upon administration.

The oligonucleotide conjugates and branched oligonucleotides described herein promote simple, efficient, non-toxic delivery of oligonucleotides (e.g., siRNA), and promote potent silencing of therapeutic targets in a range of eye cell types in vivo.

Unless otherwise specified, nomenclature used in connection with cell and tissue culture, molecular biology, immunology, microbiology, genetics and protein and nucleic acid chemistry and hybridization described herein are those well-known and commonly used in the art. Unless otherwise specified, the methods and techniques provided herein are performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

As used herein in the context of oligonucleotide sequences, “A” represents a nucleoside comprising the base adenine (e.g., adenosine or a chemically-modified derivative thereof), “G” represents a nucleoside comprising the base guanine (e.g., guanosine or a chemically-modified derivative thereof), “U” represents a nucleoside comprising the base uracil (e.g., uridine or a chemically-modified derivative thereof), and “C” represents a nucleoside comprising the base adenine (e.g., cytidine or a chemically-modified derivative thereof).

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. The siRNA is a duplex formed by a sense strand and antisense strand which have sufficient complementarity to each other to form said duplex. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” or “chemically modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 August 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C₁-C₆ alkyl, alkenyl, alkynyl, aryl, etc. Other possible modifications include those described in U.S. Pat. Nos. 5,858,988, and 6,291,438. In certain embodiments, the nucleotide analog comprises a 2′-O-methyl modification. In certain embodiments, the nucleotide analog comprises a 2′-fluoro modification.

The phosphate group of the nucleotide may also be modified, e.g., by substituting one or more of the oxygens of the phosphate group with sulfur (e.g., phosphorothioate), or by making other substitutions, which allow the nucleotide to perform its intended function, such as described in, for example, Eckstein, Antisense Nucleic Acid Drug Dev. 2000 April 10(2):117-21, Rusckowski et al. Antisense Nucleic Acid Drug Dev. 2000 October 10(5):333-45, Stein, Antisense Nucleic Acid Drug Dev. 2001 October 11(5): 317-25, Vorobjev et al. Antisense Nucleic Acid Drug Dev. 2001 April 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “oligonucleotide” refers to a short polymer of nucleotides and/or nucleotide analogs. The term “oligonucleotide” includes, but is not limited to, antisense oligonucleotide (ASO), siRNA, and micro-RNA.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g., RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, a “target” refers to a particular nucleic acid sequence (e.g., a gene, an mRNA, a miRNA or the like) that an oligonucleotide conjugate or branched oligonucleotide of the disclosure binds to and/or otherwise effects the expression of. In certain embodiments, the target is expressed in the eye. In certain embodiments, target is expressed in a specific eye cell. In other embodiments, a target is associated with a particular disease or disorder in a subject.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g., mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g., an orthologue or paralogue) of the target gene.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, antisense oligonucleotides, GAPMER molecules, short hairpin RNA (shRNA), and dual-function oligonucleotides, as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and 2,2N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also known in the art as “small temporal RNAs” (“stRNAs”), refers to a small (10-50 nucleotide) RNA, which are genetically encoded (e.g., by viral, mammalian, or plant genomes) and are capable of directing or mediating RNA silencing. An “miRNA disorder” shall refer to a disease or disorder characterized by an aberrant expression or activity of a miRNA.

As used herein, the term “dual functional oligonucleotide” refers to an RNA silencing agent having the formula T-L-μ, wherein T is an mRNA targeting moiety, L is a linking moiety, and μ is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA).

As used herein, the term “linking moiety” or “linking portion” refers to a domain, portion or region of the RNA-silencing agent which covalently joins or links the mRNA.

As used herein, the term “antisense strand” of an RNA silencing agent, e.g., an siRNA, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like, between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g., certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moiety.

As used herein, the terms “sufficient complementarity” or “sufficient degree of complementarity” mean that the RNA silencing agent has a sequence (e.g., in the antisense strand, mRNA targeting moiety or miRNA recruiting moiety), which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

Various methodologies of the instant disclosure include a step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

In one aspect, instead of the RNA silencing agent being an interfering ribonucleic acid, e.g., an siRNA or shRNA as described above, the RNAi agent can encode an interfering ribonucleic acid, e.g., an shRNA, as described above. In other words, the RNAi agent can be a transcriptional template of the interfering ribonucleic acid. Thus, RNAi agents of the present disclosure can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21-23 nucleotides (Brummelkamp et al., 2002; Lee et al., 2002, Supra; Miyagishi et al., 2002; Paddison et al., 2002, supra; Paul et al., 2002, supra; Sui et al., 2002 supra; Yu et al., 2002, supra. More information about shRNA design and use can be found on the internet at the following addresses: katandin.cshl org:9331/RNAi/docs/BseRI-BamHI_Strategy.pdf and katandin.cshl.org:9331/RNAi/docs/Web_version_of_PCR_strategy1.pdf).

Expression constructs of the present disclosure include any construct suitable for use in the appropriate expression system and include, but are not limited to, retroviral vectors, linear expression cassettes, plasmids and viral or virally-derived vectors, as known in the art. Such expression constructs can include one or more inducible promoters, RNA Pol III promoter systems, such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct. (Tuschl, T., 2002, Supra).

Synthetic siRNAs can be delivered into cells by methods known in the art, including cationic liposome transfection and electroporation. To obtain longer term suppression of the target genes (e.g., S6K1 gene) and to facilitate delivery under certain circumstances, one or more siRNA can be expressed within cells from recombinant DNA constructs. Such methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl, T., 2002, supra) capable of expressing functional double-stranded siRNAs; (Bagella et al., 1998; Lee et al., 2002, supra; Miyagishi et al., 2002, supra; Paul et al., 2002, supra; Yu et al., 2002, supra; Sui et al., 2002, supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al., 1998; Lee et al., 2002, supra; Miyagishi et al., 2002, supra; Paul et al., 2002, supra; Yu et al., 2002), supra; Sui et al., 2002, supra). Constructs containing siRNA sequence under the control of T7 promoter also make functional siRNAs when co-transfected into the cells with a vector expressing T7 RNA polymerase (Jacque et al., 2002, supra). A single construct may contain multiple sequences coding for siRNAs, such as multiple regions of the gene encoding S6K1, targeting the same gene or multiple genes, and can be driven, for example, by separate PolIII promoter sites.

Animal cells express a range of noncoding RNAs of approximately 22 nucleotides termed micro-RNA (miRNAs), which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with sequence complementary to the target mRNA, a vector construct that expresses the engineered precursor can be used to produce siRNAs to initiate RNAi against specific mRNA targets in mammalian cells (Zeng et al., 2002, supra). When expressed by DNA vectors containing polymerase III promoters, micro-RNA designed hairpins can silence gene expression (McManus et al., 2002, supra). MicroRNAs targeting polymorphisms may also be useful for blocking translation of mutant proteins, in the absence of siRNA-mediated gene-silencing. Such applications may be useful in situations, for example, where a designed siRNA caused off-target silencing of wild type protein.

Viral-mediated delivery mechanisms can also be used to induce specific silencing of targeted genes through expression of siRNA, for example, by generating recombinant adenoviruses harboring siRNA under RNA Pol II promoter transcription control (Xia et al., 2002, supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., 2002). In adult mice, efficient delivery of siRNA can be accomplished by “high-pressure” delivery technique, a rapid injection (within 5 seconds) of a large volume of siRNA containing solution into animal via the tail vein (Liu et al., 1999, supra; McCaffrey et al., 2002, supra; Lewis et al., 2002. Nanoparticles and liposomes can also be used to deliver siRNA into animals. In certain exemplary embodiments, recombinant adeno-associated viruses (rAAVs) and their associated vectors can be used to deliver one or more siRNAs into cells, e.g., neural cells (e.g., brain cells) (US Patent Applications 2014/0296486, 2010/0186103, 2008/0269149, 2006/0078542 and 2005/0220766).

The nucleic acid compositions of the disclosure include both unmodified siRNAs and modified siRNAs, such as crosslinked siRNA derivatives or derivatives having non-nucleotide moieties linked, for example to their 3′ or 5′ ends. Modifying siRNA derivatives in this way may improve cellular uptake or enhance cellular targeting activities of the resulting siRNA derivative, as compared to the corresponding siRNA, and are useful for tracing the siRNA derivative in the cell, or improving the stability of the siRNA derivative compared to the corresponding siRNA.

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA, which will be targeted by the siRNA generated from the engineered RNA precursor, and will be depleted from the cell or organism, leading to a decrease in the concentration of the protein encoded by that mRNA in the cell or organism. The RNA precursors are typically nucleic acid molecules that individually encode either one strand of a dsRNA or encode the entire nucleotide sequence of an RNA hairpin loop structure.

The nucleic acid compositions of the disclosure can be unconjugated or can be conjugated to another moiety, such as a nanoparticle, to enhance a property of the compositions, e.g., a pharmacokinetic parameter such as absorption, efficacy, bioavailability and/or half-life. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present disclosure can also be labeled using any method known in the art. For instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P or another appropriate isotope.

Moreover, because RNAi is believed to progress via at least one single-stranded RNA intermediate, the skilled artisan will appreciate that ss-siRNAs (e.g., the antisense strand of a ds-siRNA) can also be designed (e.g., for chemical synthesis), generated (e.g., enzymatically generated), or expressed (e.g., from a vector or plasmid) as described herein and utilized according to the claimed methodologies. Moreover, in invertebrates, RNAi can be triggered effectively by long dsRNAs (e.g., dsRNAs about 100-1000 nucleotides in length, such as about 200-500, for example, about 250, 300, 350, 400 or 450 nucleotides in length) acting as effectors of RNAi. (Brondani et al., Proc Natl Acad Sci USA. 2001 Dec. 4; 98(25):14428-33. Epub 2001 Nov. 27.)

### S6K1—Targeting Oligonucleotides

Ribosomal protein S6 kinase B1 (RPS6KB1), also known as S6K1, is a serine/threonine kinase that phosphorylates the S6 ribosomal protein to stimulate protein synthesis. The S6K1 gene is provided in NCBI Reference Sequence NG 029513.1. Described herein are oligonucleotides (e.g., siRNA, antisense oligonucleotides, short hairpin RNA (shRNA)) that target S6K1 mRNA and effectively silence S6K1 expression.

In one aspect, the disclosure provides an siRNA comprising a sense strand and an antisense strand, wherein the antisense strand comprises a sequence substantially complementary to a S6K1 nucleic acid sequence of any one of SEQ ID NOs: 1-6.

In one aspect, the disclosure provides an siRNA comprising a sense strand and an antisense strand, wherein the antisense strand comprises a sequence substantially complementary to a S6K1 nucleic acid sequence of SEQ ID NO: 1.

In one aspect, the disclosure provides an siRNA comprising a sense strand and an antisense strand, wherein the antisense strand comprises a sequence substantially complementary to a S6K1 nucleic acid sequence of SEQ ID NO: 2.

In one aspect, the disclosure provides an siRNA comprising a sense strand and an antisense strand, wherein the antisense strand comprises a sequence substantially complementary to a S6K1 nucleic acid sequence of SEQ ID NO: 3.

In one aspect, the disclosure provides an siRNA comprising a sense strand and an antisense strand, wherein the antisense strand comprises a sequence substantially complementary to a S6K1 nucleic acid sequence of SEQ ID NO: 4.

In one aspect, the disclosure provides an siRNA comprising a sense strand and an antisense strand, wherein the antisense strand comprises a sequence substantially complementary to a S6K1 nucleic acid sequence of SEQ ID NO: 5.

In one aspect, the disclosure provides an siRNA comprising a sense strand and an antisense strand, wherein the antisense strand comprises a sequence substantially complementary to a S6K1 nucleic acid sequence of SEQ ID NO: 6.

In certain embodiments, the antisense strand comprises a sequence substantially complementary to a S6K1 nucleic acid sequence of any one of SEQ ID NOs: 7-12. In certain embodiments, the antisense strand comprises a sequence substantially complementary to a

S6K1 nucleic acid sequence of SEQ ID NO: 7. In certain embodiments, the antisense strand comprises a sequence substantially complementary to a S6K1 nucleic acid sequence of SEQ ID NO: 8. In certain embodiments, the antisense strand comprises a sequence substantially complementary to a S6K1 nucleic acid sequence of SEQ ID NO: 9. In certain embodiments, the antisense strand comprises a sequence substantially complementary to a S6K1 nucleic acid sequence of SEQ ID NO: 10. In certain embodiments, the antisense strand comprises a sequence substantially complementary to a S6K1 nucleic acid sequence of SEQ ID NO: 11. In certain embodiments, the antisense strand comprises a sequence substantially complementary to a S6K1 nucleic acid sequence of SEQ ID NO: 12.

In certain embodiments, the siRNA comprises complementarity to at least 10, 11, 12 or 13 contiguous nucleotides of the S6K1 nucleic acid sequence of any one of SEQ ID NOs: 1-6.

In certain embodiments, the siRNA comprises no more than 3 mismatches with the S6K1 nucleic acid sequence of any one of SEQ ID NOs: 1-6.

In certain embodiments, the siRNA comprises full complementarity to the S6K1 nucleic acid sequence of any one of SEQ ID NOs: 1-6.

In certain embodiments, the siRNA comprises a sense strand and an antisense strand. In certain embodiments, the antisense strand comprises about 15 nucleotides to 25 nucleotides in length (e.g., 15, 16, 17, 18, 19, 20, 21, 22, 23, 24, or 25 nucleotides in length). In certain embodiments, the antisense strand is 20 nucleotides in length, 21 nucleotides in length, or 22 nucleotides in length. In certain embodiments, the sense strand comprises about 15 nucleotides to 25 nucleotides in length (e.g., 15, 16, 17, 18, 19, 20, 21, 22, 23, 24, or 25 nucleotides in length). In certain embodiments, the sense strand is 15 nucleotides in length, 16 nucleotides in length, 18 nucleotides in length, or 20 nucleotides in length.

In certain embodiments, the siRNA comprises a double-stranded region of 15 base pairs to 20 base pairs (e.g., 15, 16, 17, 18, 19, or 20 base pairs). In certain embodiments, the siRNA comprises a double-stranded region of 15 base pairs, 16 base pairs, 18 base pairs, or 20 base pairs.

In certain embodiments, the siRNA comprises at least one blunt-end. In certain embodiments, the siRNA comprises two blunt-ends.

In certain embodiments, the siRNA comprises at least one single stranded nucleotide overhang (also referred to herein as a “single-stranded tail”). In certain embodiments, the siRNA comprises two single stranded nucleotide overhangs. In certain embodiments, the siRNA comprises about a 2-nucleotide to 5-nucleotide single stranded nucleotide overhang (e.g., a 2-, 3-, 4-, or 5-nucleotide overhang). In certain embodiments, the siRNA comprises a 2-nucleotide single stranded nucleotide overhang or a 5-nucleotide single stranded nucleotide overhang.

In certain embodiments, the siRNA comprises naturally occurring nucleotides (i.e., unmodified ribonucleotides).

In certain embodiments, the siRNA comprises at least one modified nucleotide. In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified intemucleotide linkage. In certain embodiments, the modified intemucleotide linkage comprises a phosphorothioate intemucleotide linkage. In certain embodiments, the siRNA comprises 4-16 phosphorothioate intemucleotide linkages. In certain embodiments, the siRNA comprises 8-13 phosphorothioate intemucleotide linkages.

In certain embodiments, the siRNA comprises at least 80% chemically modified nucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% chemically modified nucleotides). In certain embodiments, the siRNA is fully chemically modified.

In certain embodiments, the siRNA comprises at least 70% 2′-O-methyl nucleotide modifications (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the antisense strand comprises at least 70% 2′-O-methyl nucleotide modifications (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the antisense strand comprises about 70% to 90% 2′-O-methyl nucleotide modifications. In certain embodiments, the sense strand comprises at least 65% 2′-O-methyl nucleotide modifications (e.g., 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the sense strand comprises 100% 2′-O-methyl nucleotide modifications.

In certain embodiments, the sense strand comprises one or more nucleotide mismatches between the antisense strand and the sense strand.

In certain embodiments, the antisense strand comprises a 5′ phosphate, a 5′-alkyl phosphonate, a 5′ alkylene phosphonate, or a 5′ alkenyl phosphonate. In certain embodiments, the antisense strand comprises a 5′ vinyl phosphonate.

### Anti-S6K1 Short Hairpin RNA (shRNA) Molecules

In certain featured embodiments, the instant disclosure provides shRNAs capable of mediating RNA silencing of an S6K1 target sequence with enhanced selectivity. In contrast to siRNAs, shRNAs mimic the natural precursors of micro RNAs (miRNAs) and enter at the top of the gene silencing pathway. For this reason, shRNAs are believed to mediate gene silencing more efficiently by being fed through the entire natural gene silencing pathway.

miRNAs are noncoding RNAs of approximately 22 nucleotides, which can regulate gene expression at the post transcriptional or translational level during plant and animal development. One common feature of miRNAs is that they are all excised from an approximately 70 nucleotide precursor RNA stem-loop termed pre-miRNA, probably by Dicer, an RNase III-type enzyme, or a homolog thereof. Naturally-occurring miRNA precursors (pre-miRNA) have a single strand that forms a duplex stem including two portions that are generally complementary, and a loop, which connects the two portions of the stem. In typical pre-miRNAs, the stem includes one or more bulges, e.g., extra nucleotides that create a single nucleotide “loop” in one portion of the stem, and/or one or more unpaired nucleotides that create a gap in the hybridization of the two portions of the stem to each other. Short hairpin RNAs, or engineered RNA precursors, of the present application are artificial constructs based on these naturally occurring pre-miRNAs, but which are engineered to deliver desired RNA silencing agents (e.g., siRNAs of the disclosure). By substituting the stem sequences of the pre-miRNA with sequence complementary to the target mRNA, a shRNA is formed. The shRNA is processed by the entire gene silencing pathway of the cell, thereby efficiently mediating RNAi.

The requisite elements of a shRNA molecule include a first portion and a second portion, having sufficient complementarity to anneal or hybridize to form a duplex or double-stranded stem portion. The two portions need not be fully or perfectly complementary. The first and second “stem” portions are connected by a portion having a sequence that has insufficient sequence complementarity to anneal or hybridize to other portions of the shRNA. This latter portion is referred to as a “loop” portion in the shRNA molecule. The shRNA molecules are processed to generate siRNAs. shRNAs can also include one or more bulges, i.e., extra nucleotides that create a small nucleotide “loop” in a portion of the stem, for example a one-, two- or three-nucleotide loop. The stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

In shRNAs (or engineered precursor RNAs) of the instant disclosure, one portion of the duplex stem is a nucleic acid sequence that is complementary (or anti-sense) to the APP target sequence. In certain embodiments, one strand of the stem portion of the shRNA is sufficiently complementary (e.g., antisense) to a target RNA (e.g., mRNA) sequence to mediate degradation or cleavage of said target RNA via RNA interference (RNAi). Thus, engineered RNA precursors include a duplex stem with two portions and a loop connecting the two stem portions. The antisense portion can be on the 5′ or 3′ end of the stem. The stem portions of a shRNA are about 15 to about 50 nucleotides in length. In certain embodiments, the two stem portions are about 18 or 19 to about 21, 22, 23, 24, 25, 30, 35, 37, 38, 39, or 40 or more nucleotides in length. In certain embodiments, the length of the stem portions should be 21 nucleotides or greater. When used in mammalian cells, the length of the stem portions should be less than about 30 nucleotides to avoid provoking non-specific responses like the interferon pathway. In non-mammalian cells, the stem can be longer than 30 nucleotides. In fact, the stem can include much larger sections complementary to the target mRNA (up to, and including the entire mRNA). In fact, a stem portion can include much larger sections complementary to the target mRNA (up to, and including the entire mRNA).

The two portions of the duplex stem must be sufficiently complementary to hybridize to form the duplex stem. Thus, the two portions can be, but need not be, fully or perfectly complementary. In addition, the two stem portions can be the same length, or one portion can include an overhang of 1, 2, 3, or 4 nucleotides. The overhanging nucleotides can include, for example, uracils (Us), e.g., all Us. The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop in the shRNAs or engineered RNA precursors can be 2, 3, 4, 5, 6, 7, 8, 9, or more, e.g., 15 or 20, or more nucleotides in length.

The loop in the shRNAs or engineered RNA precursors may differ from natural pre-miRNA sequences by modifying the loop sequence to increase or decrease the number of paired nucleotides, or replacing all or part of the loop sequence with a tetraloop or other loop sequences. Thus, the loop portion in the shRNA can be about 2 to about 20 nucleotides in length, i.e., about 2, 3, 4, 5, 6, 7, 8, 9, or more, e.g., 15 or 20, or more nucleotides in length. In certain embodiments, a loop consists of or comprises a “tetraloop” sequence. Exemplary tetraloop sequences include, but are not limited to, the sequences GNRA, where N is any nucleotide and R is a purine nucleotide, GGGG, and UUUU.

In certain embodiments, shRNAs of the present application include the sequences of a desired siRNA molecule described supra. In other embodiments, the sequence of the antisense portion of a shRNA can be designed essentially as described above or generally by selecting an 18, 19, 20, 21 nucleotide, or longer, sequence from within the target RNA (e.g., APP mRNA), for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR. This sequence can optionally follow immediately after a region of the target gene containing two adjacent AA nucleotides. The last two nucleotides of the nucleotide sequence can be selected to be UU. This 21 or so nucleotide sequence is used to create one portion of a duplex stem in the shRNA. This sequence can replace a stem portion of a wild-type pre-miRNA sequence, e.g., enzymatically, or is included in a complete sequence that is synthesized. For example, one can synthesize DNA oligonucleotides that encode the entire stem-loop engineered RNA precursor, or that encode just the portion to be inserted into the duplex stem of the precursor, and using restriction enzymes to build the engineered RNA precursor construct, e.g., from a wild-type pre-miRNA.

Engineered RNA precursors include, in the duplex stem, the 21-22 or so nucleotide sequences of the siRNA or siRNA-like duplex desired to be produced in vivo. Thus, the stem portion of the engineered RNA precursor includes at least 18 or 19 nucleotide pairs corresponding to the sequence of an exonic portion of the gene whose expression is to be reduced or inhibited. The two 3′ nucleotides flanking this region of the stem are chosen so as to maximize the production of the siRNA from the engineered RNA precursor and to maximize the efficacy of the resulting siRNA in targeting the corresponding mRNA for translational repression or destruction by RNAi in vivo and in vitro.

In certain embodiments, shRNAs of the disclosure include miRNA sequences, optionally end-modified miRNA sequences, to enhance entry into RISC. The miRNA sequence can be similar or identical to that of any naturally occurring miRNA (see e.g. The miRNA Registry; Griffiths-Jones S, Nuc. Acids Res., 2004). Over one thousand natural miRNAs have been identified to date and together they are thought to comprise about 1% of all predicted genes in the genome. Many natural miRNAs are clustered together in the introns of pre-mRNAs and can be identified in silico using homology-based searches (Pasquinelli et al., 2000; Lagos-Quintana et al., 2001; Lau et al., 2001; Lee and Ambros, 2001) or computer algorithms (e.g. MiRScan, MiRSeeker) that predict the capability of a candidate miRNA gene to form the stem loop structure of a pri-mRNA (Grad et al., Mol. Cell., 2003; Lim et al., Genes Dev., 2003; Lim et al., Science, 2003; Lai E C et al., Genome Bio., 2003). An online registry provides a searchable database of all published miRNA sequences (The miRNA Registry at the Sanger Institute website; Griffiths-Jones S, Nuc. Acids Res., 2004). Exemplary, natural miRNAs include lin-4, let-7, miR-10, mirR-15, miR-16, miR-168, miR-175, miR-196 and their homologs, as well as other natural miRNAs from humans and certain model organisms including Drosophila melanogaster, Caenorhabditis elegans, zebrafish, Arabidopsis thalania, Mus musculus, and Rattus norvegicus as described in International PCT Publication No. WO 03/029459.

Naturally-occurring miRNAs are expressed by endogenous genes in vivo and are processed from a hairpin or stem-loop precursor (pre-miRNA or pri-miRNAs) by Dicer or other RNAses (Lagos-Quintana et al., Science, 2001; Lau et al., Science, 2001; Lee and Ambros, Science, 2001; Lagos-Quintana et al., Curr. Biol., 2002; Mourelatos et al., Genes Dev., 2002; Reinhart et al., Science, 2002; Ambros et al., Curr. Biol., 2003; Brennecke et al., 2003; Lagos-Quintana et al., RNA, 2003; Lim et al., Genes Dev., 2003; Lim et al., Science, 2003). miRNAs can exist transiently in vivo as a double-stranded duplex, but only one strand is taken up by the RISC complex to direct gene silencing. Certain miRNAs, e.g., plant miRNAs, have perfect or near-perfect complementarity to their target mRNAs and, hence, direct cleavage of the target mRNAs. Other miRNAs have less than perfect complementarity to their target mRNAs and, hence, direct translational repression of the target mRNAs. The degree of complementarity between a miRNA and its target mRNA is believed to determine its mechanism of action. For example, perfect or near-perfect complementarity between a miRNA and its target mRNA is predictive of a cleavage mechanism (Yekta et al., Science, 2004), whereas less than perfect complementarity is predictive of a translational repression mechanism. In certain embodiments, the miRNA sequence is that of a naturally-occurring miRNA sequence, the aberrant expression or activity of which is correlated with a miRNA disorder.

### Modified Anti-S6K1 RNA Silencing Agents

In certain aspects of the disclosure, an RNA silencing agent (or any portion thereof) of the present application, as described supra, may be modified, such that the activity of the agent is further improved. For example, the RNA silencing agents described in Section II supra, may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the present application may be substituted with a destabilizing nucleotide to enhance single nucleotide target discrimination (see U.S. application Ser. No. 11/698,689, filed Jan. 25, 2007, and U.S. Provisional Application No. 60/762,225 filed Jan. 25, 2006, both of which are incorporated herein by reference). Such a modification may be sufficient to abolish the specificity of the RNA silencing agent for a non-target mRNA (e.g., wild-type mRNA), without appreciably affecting the specificity of the RNA silencing agent for a target mRNA (e.g., gain-of-function mutant mRNA).

In certain embodiments, the RNA silencing agents of the present application are modified by the introduction of at least one universal nucleotide in the antisense strand thereof. Universal nucleotides comprise base portions that are capable of base pairing indiscriminately with any of the four conventional nucleotide bases (e.g., A, G, C, U). A universal nucleotide is contemplated because it has relatively minor effect on the stability of the RNA duplex or the duplex formed by the guide strand of the RNA silencing agent and the target mRNA. Exemplary universal nucleotides include those having an inosine base portion or an inosine analog base portion selected from the group consisting of deoxyinosine (e.g., 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In certain embodiments, the universal nucleotide is an inosine residue or a naturally occurring analog thereof.

In certain embodiments, the RNA silencing agents of the disclosure are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destabilizing nucleotide and the specificity-determining nucleotide). In RNA silencing agents having two strands or strand portions (e.g., siRNAs and shRNAs), the destabilizing nucleotide may be introduced in the strand or strand portion that does not contain the specificity-determining nucleotide. In certain embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the disclosure may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the present application or an siRNA produced from a shRNA) into RISC in favor of the sense strand, such that the antisense strand preferentially guides cleavage or translational repression of a target mRNA, and thus increasing or improving the efficiency of target cleavage and silencing. In certain embodiments, the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the present application may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the disclosure may be enhanced such that there is at least one mismatched base pair between the 5′ end of the first or antisense strand and the 3′ end of the sense strand portion. In certain embodiments, the mismatched base pair is selected from the group consisting of G:A, C:A, C:U, G:G, A:A, C:C and U:U. In another embodiment, the asymmetry of an RNA silencing agent of the disclosure may be enhanced such that there is at least one wobble base pair, e.g., G:U, between the 5′ end of the first or antisense strand and the 3′ end of the sense strand portion. In another embodiment, the asymmetry of an RNA silencing agent of the disclosure may be enhanced such that there is at least one base pair comprising a rare nucleotide, e.g., inosine (I). In certain embodiments, the base pair is selected from the group consisting of an I:A, I:U and I:C. In yet another embodiment, the asymmetry of an RNA silencing agent of the disclosure may be enhanced such that there is at least one base pair comprising a modified nucleotide. In certain embodiments, the modified nucleotide is selected from the group consisting of 2-amino-G, 2-amino-A, 2,6-diamino-G, and 2,6-diamino-A.

3) RNA Silencing Agents with Enhanced Stability

The RNA silencing agents of the present application can be modified to improve stability in serum or in growth medium for cell cultures. In order to enhance the stability, the 3′-residues may be stabilized against degradation, e.g., they may be selected such that they consist of purine nucleotides, such as adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a one aspect, the present application features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In one aspect, the present application features RNA silencing agents that are at least 80% chemically modified. In certain embodiments, the RNA silencing agents may be fully chemically modified, i.e., 100% of the nucleotides are chemically modified. In another aspect, the present application features RNA silencing agents comprising 2′-OH ribose groups that are at least 80% chemically modified. In certain embodiments, the RNA silencing agents comprise 2′-OH ribose groups that are about 80%, 85%, 90%, 95%, or 100% chemically modified.

In certain embodiments, the RNA silencing agents may contain at least one modified nucleotide analogue. The nucleotide analogues may be located at positions where the target-specific silencing activity, e.g., the RNAi mediating activity or translational repression activity is not substantially affected, e.g., in a region at the 5′-end and/or the 3′-end of the siRNA molecule. Moreover, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In certain embodiments, the modifications are 2′-fluoro, 2′-amino and/or 2′-thio modifications. Modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a certain embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agent moieties of the instant disclosure. Additional modified residues include, deoxy-abasic, inosine, N3-methyl-uridine, N6,N6-dimethyl-adenosine, pseudouridine, purine ribonucleoside and ribavirin. In a certain embodiment, the 2′ moiety is a methyl group such that the linking moiety is a 2′-O-methyl oligonucleotide.

In a certain embodiment, the RNA silencing agent of the present application comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-0,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the present application comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone ,which is highly resistant to nuclease digestion and imparts improved binding specificity to the molecule (Nielsen, et al., Science, (2001), 254: 1497-1500).

Also contemplated are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the present application includes RNA silencing agents having two complementary strands of nucleic acid, wherein the two strands are crosslinked. The present application also includes RNA silencing agents which are conjugated or unconjugated (e.g., at its 3′ terminus) to another moiety (e.g., a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′ OMe moiety on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a O with an S; (c) replacement of the U with a C5 amino linker; (d) replacement of an A with a G (sequence changes can be located on the sense strand and not the antisense strand in certain embodiments); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a O with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### Heavily Modified RNA Silencing Agents

In certain embodiments, the RNA silencing agent comprises at least 80% chemically modified nucleotides. In certain embodiments, the RNA silencing agent is fully chemically modified, i.e., 100% of the nucleotides are chemically modified.

In certain embodiments, the RNA silencing agent is 2′-O-methyl rich, i.e., comprises greater than 50% 2′-O-methyl content. In certain embodiments, the RNA silencing agent comprises at least about 55%, 60%, 65%, 70%, 75%, 80%, 85%, 90%, 95%, or 100% 2′-O-methyl nucleotide content. In certain embodiments, the RNA silencing agent comprises at least about 70% 2′-O-methyl nucleotide modifications. In certain embodiments, the RNA silencing agent comprises between about 70% and about 90% 2′-O-methyl nucleotide modifications. In certain embodiments, the RNA silencing agent is a dsRNA comprising an antisense strand and sense strand. In certain embodiments, the antisense strand comprises at least about 70% 2′-O-methyl nucleotide modifications. In certain embodiments, the antisense strand comprises between about 70% and about 90% 2′-O-methyl nucleotide modifications. In certain embodiments, the sense strand comprises at least about 70% 2′-O-methyl nucleotide modifications. In certain embodiments, the sense strand comprises between about 70% and about 90% 2′-O-methyl nucleotide modifications. In certain embodiments, the sense strand comprises between 100% 2′-O-methyl nucleotide modifications.

2′-O-methyl rich RNA silencing agents and specific chemical modification patterns are further described in US20200087663 and US20210115442, each of which is incorporated herein by reference.

### Intemucleotide Linkage Modifications

In certain embodiments, at least one intemucleotide linkage, intersubunit linkage, or nucleotide backbone is modified in the RNA silencing agent. In certain embodiments, all of the intemucleotide linkages in the RNA silencing agent are modified. In certain embodiments, the modified intemucleotide linkage comprises a phosphorothioate intemucleotide linkage. In certain embodiments, the RNA silencing agent comprise 1, 2, 3, 4, 5, 6, 7, 8, 9, 10, 11, 12, 13, 14, 15, 16, 17, 18, 19, 20, 21, 22, 23, 24, or 25 phosphorothioate intemucleotide linkages. In certain embodiments, the RNA silencing agent comprises 4-16 phosphorothioate intemucleotide linkages. In certain embodiments, the RNA silencing agent comprises 8-13 phosphorothioate intemucleotide linkages. In certain embodiments, the RNA silencing agent is a dsRNA comprising an antisense strand and a sense strand, each comprising a 5′ end and a 3′ end. In certain embodiments, the nucleotides at positions 1 and 2 from the 5′ end of sense strand are connected to adjacent ribonucleotides via phosphorothioate intemucleotide linkages. In certain embodiments, the nucleotides at positions 1 and 2 from the 3′ end of sense strand are connected to adjacent ribonucleotides via phosphorothioate intemucleotide linkages. In certain embodiments, the nucleotides at positions 1 and 2 from the 5′ end of antisense strand are connected to adjacent ribonucleotides via phosphorothioate intemucleotide linkages. In certain embodiments, the nucleotides at positions 1-2 to 1-8 from the 3′ end of antisense strand are connected to adjacent ribonucleotides via phosphorothioate intemucleotide linkages. In certain embodiments, the nucleotides at positions 1-2, 1-3, 1-4, 1-5, 1-6, 1-7, or 1-8 from the 3′ end of antisense strand are connected to adjacent ribonucleotides via phosphorothioate intemucleotide linkages. In certain embodiments, the nucleotides at positions 1-2 to 1-7 from the 3′ end of antisense strand are connected to adjacent ribonucleotides via phosphorothioate intemucleotide linkages.

In one aspect, the disclosure provides a modified oligonucleotide, said oligonucleotide having a 5′ end, a 3′ end, that is complementary to a target, wherein the oligonucleotide comprises a sense and antisense strand, and at least one modified intersubunit linkage of Formula (I):

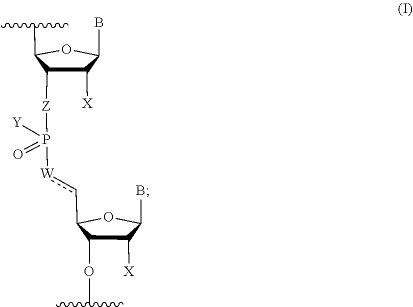

wherein:
B is a base pairing moiety;
W is selected from the group consisting of O, OCH₂, OCH, CH₂, and CH;
X is selected from the group consisting of halo, hydroxy, and C₁₋₆ alkoxy;
Y is selected from the group consisting of O⁻, OH, OR, NH⁻, NH₂, S⁻, and SH;
Z is selected from the group consisting of O and CH₂;
   R is a protecting group; and [symbol]
   is an optional double bond.

In an embodiment of Formula (I), when W is CH, [symbol] is a double bond.

In an embodiment of Formula (I), when W selected from the group consisting of O, OCH₂, OCH, CH₂, [symbol] is a single bond.

In an embodiment of Formula (I), when Y is O⁻, either Z or W is not O.

In an embodiment of Formula (I), Z is CH₂ and W is CH₂. In another embodiment, the modified intersubunit linkage of Formula (I) is a modified intersubunit linkage of Formula (II):

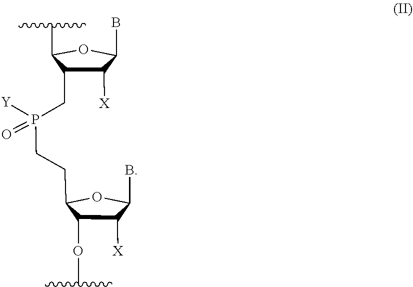

In an embodiment of Formula (I), Z is CH₂ and W is O. In another embodiment, wherein the modified intersubunit linkage of Formula (I) is a modified intersubunit linkage of Formula (III):

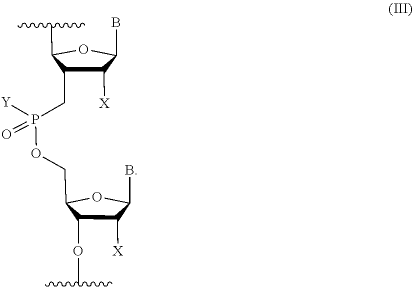

In an embodiment of Formula (I), Z is O and W is CH₂. In another embodiment, the modified intersubunit linkage of Formula (I) is a modified intersubunit linkage of Formula (IV):

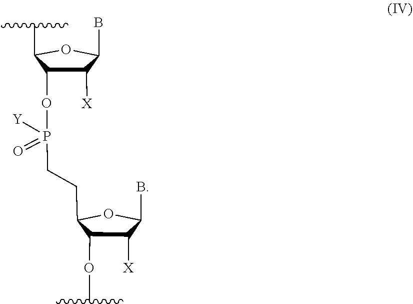

In an embodiment of Formula (I), Z is O and W is CH. In another embodiment, the modified intersubunit linkage of Formula (I) is a modified intersubunit linkage of Formula V:

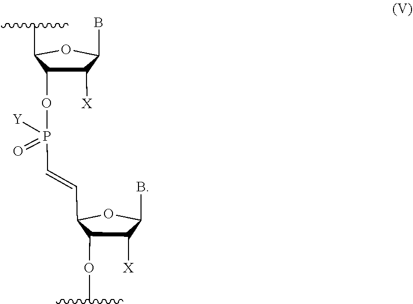

In an embodiment of Formula (I), Z is O and W is OCH₂. In another embodiment, the modified intersubunit linkage of Formula (I) is a modified intersubunit linkage of Formula VI:

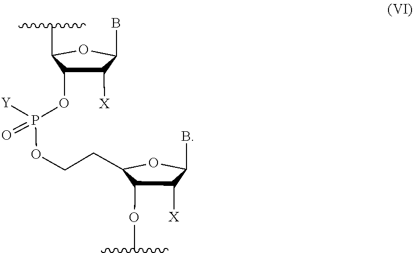

In an embodiment of Formula (I), Z is CH₂ and W is CH. In another embodiment, the modified intersubunit linkage of Formula (I) is a modified intersubunit linkage of Formula VII:

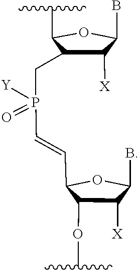

In an embodiment of Formula (I), the base pairing moiety B is selected from the group consisting of adenine, guanine, cytosine, and uracil.

In an embodiment, the modified oligonucleotide is incorporated into siRNA, said modified siRNA having a 5′ end, a 3′ end, that is complementary to a target, wherein the siRNA comprises a sense and antisense strand, and at least one modified intersubunit linkage of any one or more of Formula (I), Formula (II), Formula (III), Formula (IV), Formula (V), Formula (VI), or Formula (VII).

In an embodiment, the modified oligonucleotide is incorporated into siRNA, said modified siRNA having a 5′ end, a 3′ end, that is complementary to a target and comprises a sense and antisense strand, wherein the siRNA comprises at least one modified intersubunit linkage is of Formula VIII:

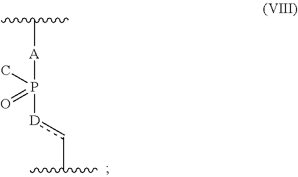

wherein:
D is selected from the group consisting of O, OCH₂, OCH, CH2, and CH;
C is selected from the group consisting of O⁻, OH, OR¹, NH⁻, NH₂, S⁻ , and SH;
A is selected from the group consisting of O and CH₂;
   R¹ is a protecting group; [symbol]
   is an optional double bond; and
   the intersubunit is bridging two optionally modified nucleosides. In an embodiment, when C is O⁻, either A or D is not O.

In an embodiment, D is CH₂. In another embodiment, the modified intersubunit linkage of Formula VIII is a modified intersubunit linkage of Formula (IX):

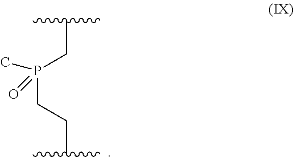 [SMILES: *CCP(C)(=O)C*]

In an embodiment, D is O. In another embodiment, the modified intersubunit linkage of Formula VIII is a modified intersubunit linkage of Formula (X):

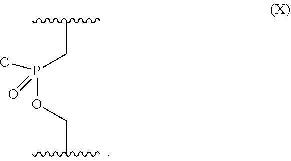 [SMILES: *COP(C)(=O)C*]

In an embodiment, D is CH₂. In another embodiment, the modified intersubunit linkage of Formula (VIII) is a modified intersubunit linkage of Formula (XI):

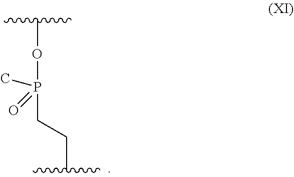 [SMILES: *CCP(C)(=O)O*]

In an embodiment, D is CH. In another embodiment, the modified intersubunit linkage of Formula VIII is a modified intersubunit linkage of Formula (XII):

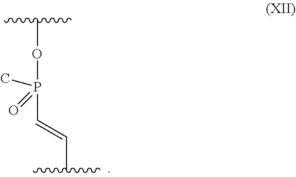 [SMILES: */C=C/P(C)(=O)O*]

In another embodiment, the modified intersubunit linkage of Formula (VII) is a modified intersubunit linkage of Formula (XIV):

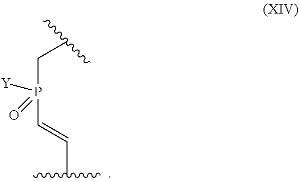

In an embodiment, D is OCH₂. In another embodiment, the modified intersubunit linkage of Formula (VII) is a modified intersubunit linkage of Formula (XIII):

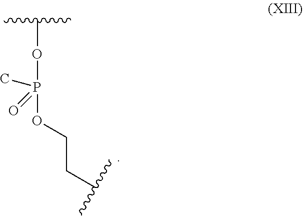 [SMILES: *CCOP(C)(=O)O*]

In another embodiment, the modified intersubunit linkage of Formula (VII) is a modified intersubunit linkage of Formula (XXa):

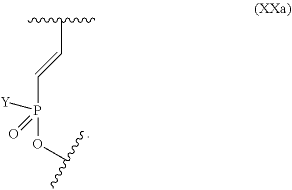

In an embodiment of the modified siRNA linkage, each optionally modified nucleoside is independently, at each occurrence, selected from the group consisting of adenosine, guanosine, cytidine, and uridine.

In certain exemplary embodiments of Formula (I), W is O. In another embodiment, W is CH₂. In yet another embodiment, W is CH.

In certain exemplary embodiments of Formula (I), X is OH. In another embodiment, X is OCH₃. In yet another embodiment, X is halo.

In a certain embodiment of Formula (I), the modified siRNA does not comprise a 2′-fluoro substituent.

In an embodiment of Formula (I), Y is O⁻. In another embodiment, Y is OH. In yet another embodiment, Y is OR. In still another embodiment, Y is NH⁻. In an embodiment, Y is NH₂. In another embodiment, Y is S. In yet another embodiment, Y is SH.

In an embodiment of Formula (I), Z is O. In another embodiment, Z is CH₂.

In an embodiment, the modified intersubunit linkage is inserted on position 1-2 of the antisense strand. In another embodiment, the modified intersubunit linkage is inserted on position 6-7 of the antisense strand. In yet another embodiment, the modified intersubunit linkage is inserted on position 10-11 of the antisense strand. In still another embodiment, the modified intersubunit linkage is inserted on position 19-20 of the antisense strand. In an embodiment, the modified intersubunit linkage is inserted on positions 5-6 and 18-19 of the antisense strand.

In an exemplary embodiment of the modified siRNA linkage of Formula (VIII), C is O⁻. In another embodiment, C is OH. In yet another embodiment, C is OR¹. In still another embodiment, C is NH⁻. In an embodiment, C is NH₂. In another embodiment, C is S⁻. In yet another embodiment, C is SH.

In an exemplary embodiment of the modified siRNA linkage of Formula (VIII), A is O. In another embodiment, A is CH₂. In yet another embodiment, C is OR¹. In still another embodiment, C is NH⁻. In an embodiment, C is NH₂. In another embodiment, C is S. In yet another embodiment, C is SH.

In a certain embodiment of the modified siRNA linkage of Formula (VIII), the optionally modified nucleoside is adenosine. In another embodiment of the modified siRNA linkage of Formula (VIII), the optionally modified nucleoside is guanosine. In another embodiment of the modified siRNA linkage of Formula (VIII), the optionally modified nucleoside is cytidine. In another embodiment of the modified siRNA linkage of Formula (VIII), the optionally modified nucleoside is uridine.

In an embodiment of the modified siRNA linkage, wherein the linkage is inserted on position 1-2 of the antisense strand. In another embodiment, the linkage is inserted on position 6-7 of the antisense strand. In yet another embodiment, the linkage is inserted on position 10-11 of the antisense strand. In still another embodiment, the linkage is inserted on position 19-20 of the antisense strand. In an embodiment, the linkage is inserted on positions 5-6 and 18-19 of the antisense strand.

In certain embodiments of Formula (I), the base pairing moiety B is adenine. In certain embodiments of Formula (I), the base pairing moiety B is guanine. In certain embodiments of Formula (I), the base pairing moiety B is cytosine. In certain embodiments of Formula (I), the base pairing moiety B is uracil.

In an embodiment of Formula (I), W is O. In an embodiment of Formula (I), W is CH₂. In an embodiment of Formula (I), W is CH.

In an embodiment of Formula (I), X is OH. In an embodiment of Formula (I), X is OCH₃. In an embodiment of Formula (I), X is halo.

In an exemplary embodiment of Formula (I), the modified oligonucleotide does not comprise a 2′-fluoro substituent.

In an embodiment of Formula (I), Y is O⁻. In an embodiment of Formula (I), Y is OH. In an embodiment of Formula (I), Y is OR. In an embodiment of Formula (I), Y is NH⁻. In an embodiment of Formula (I), Y is NH₂. In an embodiment of Formula (I), Y is S⁻. In an embodiment of Formula (I), Y is SH.

In an embodiment of Formula (I), Z is O. In an embodiment of Formula (I), Z is CH₂.

In an embodiment of the Formula (I), the linkage is inserted on position 1-2 of the antisense strand. In another embodiment of Formula (I), the linkage is inserted on position 6-7 of the antisense strand. In yet another embodiment of Formula (I), the linkage is inserted on position 10-11 of the antisense strand. In still another embodiment of Formula (I), the linkage is inserted on position 19-20 of the antisense strand. In an embodiment of Formula (I), the linkage is inserted on positions 5-6 and 18-19 of the antisense strand.

Modified intersubunit linkages are further described in U.S. Patent Publication No. 2020/0385740A1, and U.S. Patent Publication No. 2022/0010309, each of which is incorporated herein by reference.

### 4) Conjugated Functional Moieties

In other embodiments, RNA silencing agents may be modified with one or more functional moieties. A functional moiety is a molecule that confers one or more additional activities to the RNA silencing agent. In certain embodiments, the functional moieties enhance cellular uptake by target cells (e.g., neuronal cells). Thus, the disclosure includes RNA silencing agents which are conjugated or unconjugated (e.g., at its 5′ and/or 3′ terminus) to another moiety (e.g., a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a certain embodiment, the functional moiety is a hydrophobic moiety. In a certain embodiment, the hydrophobic moiety is selected from the group consisting of fatty acids, steroids, secosteroids, lipids, gangliosides and nucleoside analogs, endocannabinoids, and vitamins. In a certain embodiment, the steroid selected from the group consisting of cholesterol and lithocholic acid (LA). In a certain embodiment, the fatty acid selected from the group consisting of Eicosapentaenoic acid (EPA), Docosahexaenoic acid (DHA) and Docosanoic acid (DCA). In a certain embodiment, the vitamin selected from the group consisting of choline, vitamin A, vitamin E, and derivatives or metabolites thereof. In a certain embodiment, the vitamin is selected from the group consisting of retinoic acid and alphα-tocopheryl succinate.

In a certain embodiment, an RNA silencing agent of disclosure is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the 3′ end of the sense strand. In certain embodiments, the lipophilic moiety is selected from the group consisting of cholesterol, vitamin E, vitamin K, vitamin A, folic acid, a cationic dye (e.g., Cy3). In an exemplary embodiment, the lipophilic moiety is cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, bomeol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In certain embodiments, the functional moieties may comprise one or more ligands tethered to an RNA silencing agent to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These can be located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., O-phenanthroline), a polyamine, a tripeptide (e.g., lys-tyr-lys tripeptide), or a metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to an RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine, has an increased affinity for the HIV Rev-response element (RRE). In some embodiments, the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, either directly or indirectly, via an intervening tether, to a ligand-conjugated carrier. In certain embodiments, the coupling is through a covalent bond. In certain embodiments, the ligand is attached to the carrier via an intervening tether. In certain embodiments, a ligand alters the distribution, targeting or lifetime of an RNA silencing agent into which it is incorporated. In certain embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine (GalNAc) or derivatives thereof, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu³⁺complexes of tetraazamacrocycles), dinitrophenyl, HRP or AP. In certain embodiments, the ligand is GalNAc or a derivative thereof.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-kB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNF□), interleukin-1 beta, or gamma interferon. In one aspect, the ligand is a lipid or lipid-based molecule. Such a lipid or lipid-based molecule can bind a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid-based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a certain embodiment, the lipid-based ligand binds HSA. A lipid-based ligand can bind HSA with a sufficient affinity such that the conjugate will be distributed to a non-kidney tissue. However, it is contemplated that the affinity is not so strong that the HSA-ligand binding cannot be reversed. In another embodiment, the lipid-based ligand binds HSA weakly or not at all, such that the conjugate will be distributed to the kidney. Other moieties that target to kidney cells can also be used in place of or in addition to the lipid-based ligand.

In another aspect, the ligand is a moiety, e.g., a vitamin, which is taken up by a target cell, e.g., a proliferating cell. These can be useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low-density lipoprotein (LDL).

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an alpha-helical agent, which may have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

In certain embodiments, the functional moiety is linked to the 5′ end and/or 3′ end of the RNA silencing agent of the disclosure. In certain embodiments, the functional moiety is linked to the 5′ end and/or 3′ end of an antisense strand of the RNA silencing agent of the disclosure. In certain embodiments, the functional moiety is linked to the 5′ end and/or 3′ end of a sense strand of the RNA silencing agent of the disclosure. In certain embodiments, the functional moiety is linked to the 3′ end of a sense strand of the RNA silencing agent of the disclosure.

In certain embodiments, the functional moiety is linked to the RNA silencing agent by a linker. In certain embodiments, the functional moiety is linked to the antisense strand and/or sense strand by a linker. In certain embodiments, the functional moiety is linked to the 3′ end of a sense strand by a linker. In certain embodiments, the linker comprises a divalent or trivalent linker. In certain embodiments, the linker comprises an ethylene glycol chain, an alkyl chain, a peptide, RNA, DNA, a phosphodiester, a phosphorothioate, a phosphoramidate, an amide, a carbamate, or a combination thereof. In certain embodiments, the divalent or trivalent linker is selected from:

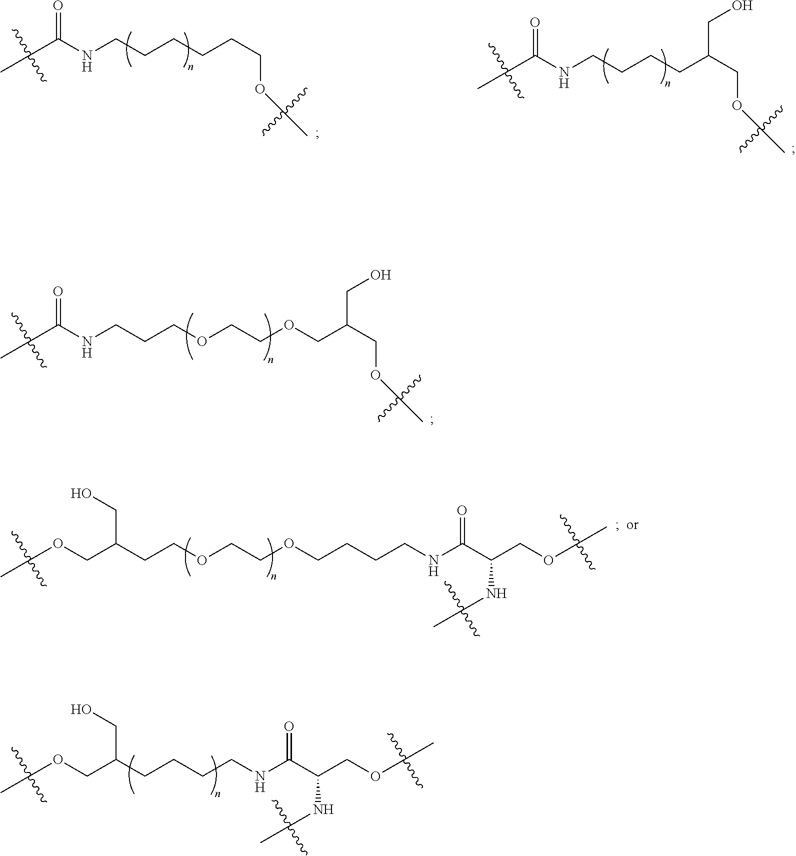

wherein n is 1, 2, 3, 4, or 5.

In certain embodiments, the linker further comprises a phosphodiester or phosphodiester derivative. In certain embodiments, the phosphodiester or phosphodiester derivative is selected from the group consisting of:

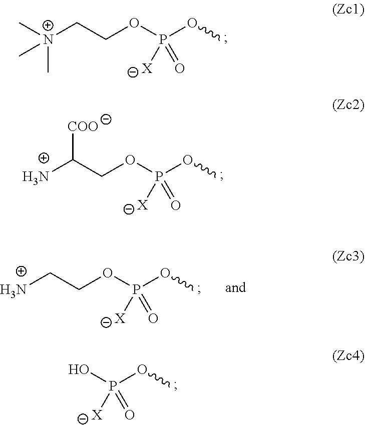

wherein X is O, S or BH₃.

The various functional moieties of the disclosure and means to conjugate them to RNA silencing agents are described in further detail in WO2017/030973A1 and WO2018/031933A2, incorporated herein by reference.

### Anti-S6K1 Oligonucleotide Conjugates For Eye Delivery

The S6K1-targeting oligonucleotides of the disclosure may be linked to a functional moiety for eye delivery. The functional moieties provide enhanced eye delivery of the oligonucleotide, including eye cell-specific delivery.

In certain embodiments, the functional moiety comprises any one of triple amine, retinoic acid (RA), docosahexaenoic acid (DHA), docosanoic acid (DCA), α-tocopheryl succinate (TS), or lithocholic acid (LA).

Each of the functional moieties described above are depicted below structurally. The functional moieties can have different isomeric configurations than the ones presented in this disclosure.

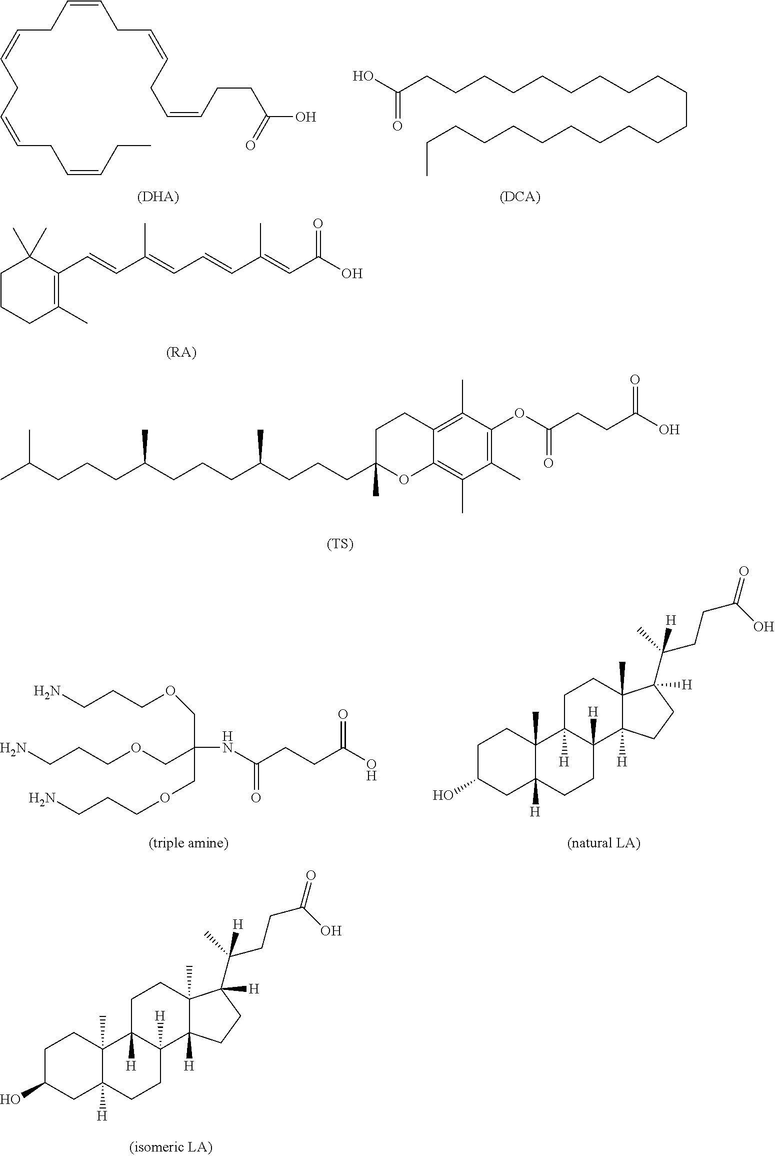

In certain embodiments, two DHA functional moieties are linked to the oligonucleotide.

In certain embodiments, the functional moiety is linked to the 5′ end and/or 3′ end of the oligonucleotide. In certain embodiments, the functional moiety is linked to the 5′ end and/or 3′ end of the sense strand or to the 5′ end and/or 3′ end of the antisense strand. In certain embodiments, the functional moiety is linked to the 3′ end of the sense strand.

In certain embodiments, the functional moiety is linked to the antisense strand and/or sense strand by a linker.

In certain embodiments, the linker comprises a divalent or trivalent linker.

In certain embodiments, the divalent or trivalent linker is selected from the group consisting of:

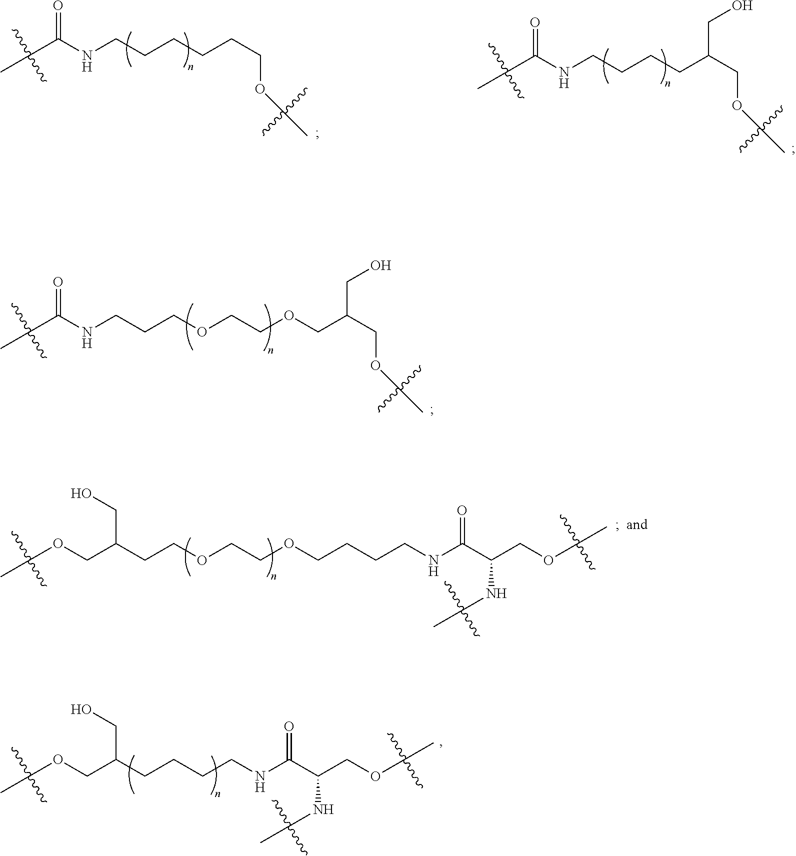

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, when the linker is a trivalent linker, the linker further links a phosphodiester or phosphodiester derivative.

In certain embodiments, the phosphodiester or phosphodiester derivative is selected from the group consisting of:

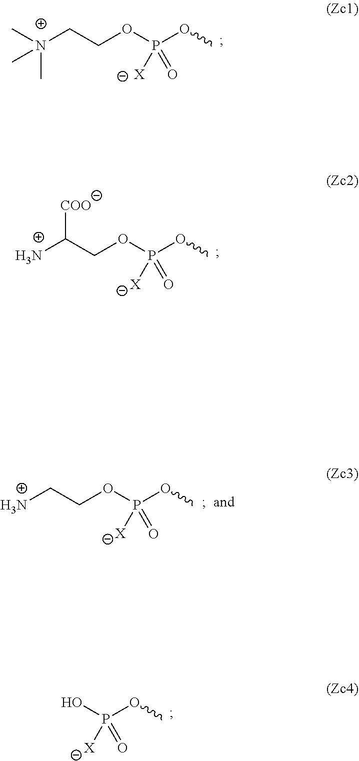

wherein X is O, S or BH₃.

The above recited moiety Zc1 is phosphatidylcholine (PC). Any one of the functional moieties described herein may comprise a phosphatidylcholine (PC) esterified derivative, i.e., phosphatidylcholine (PC) esterified triple amine (PC-triple amine), phosphatidylcholine (PC) esterified retinoic acid (PC-RA), phosphatidylcholine (PC) esterified docosahexaenoic acid (PC-DHA), phosphatidylcholine (PC) esterified docosanoic acid (PC-DCA), phosphatidylcholine (PC) esterified α-tocopheryl succinate (PC-TS), or phosphatidylcholine (PC) esterified lithocholic acid (PC-LA).

In certain embodiments, the S6K1-targeting oligonucleotide conjugate comprises the structure:

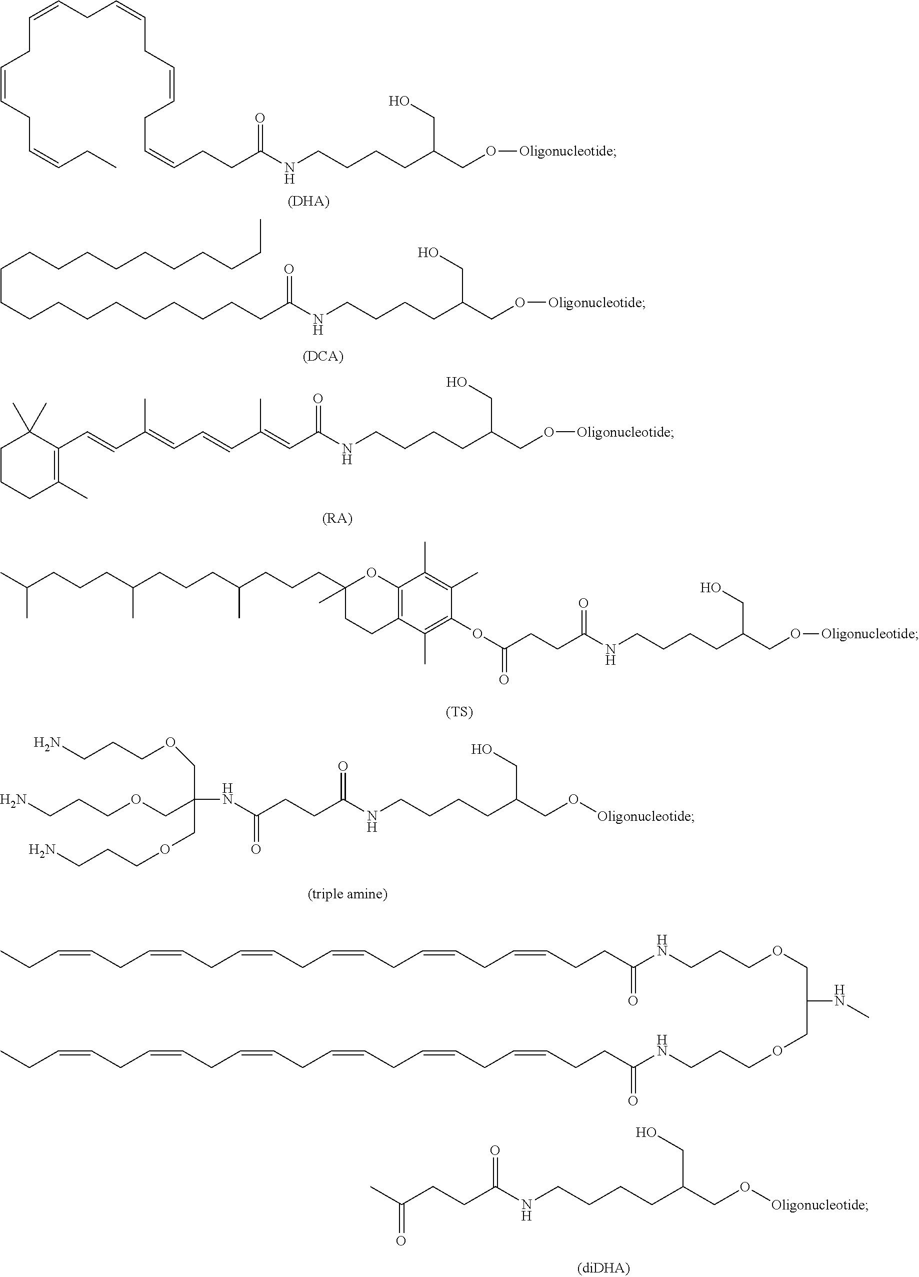

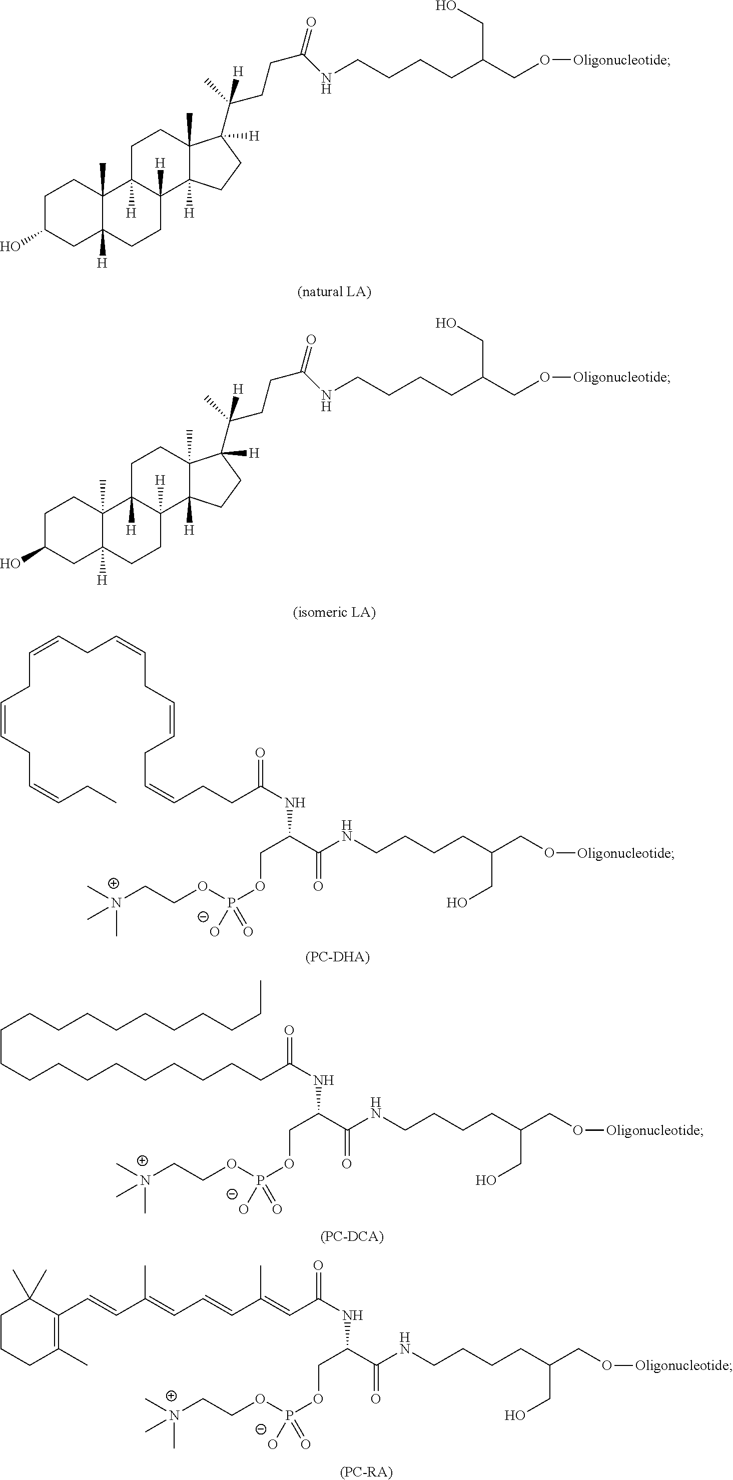

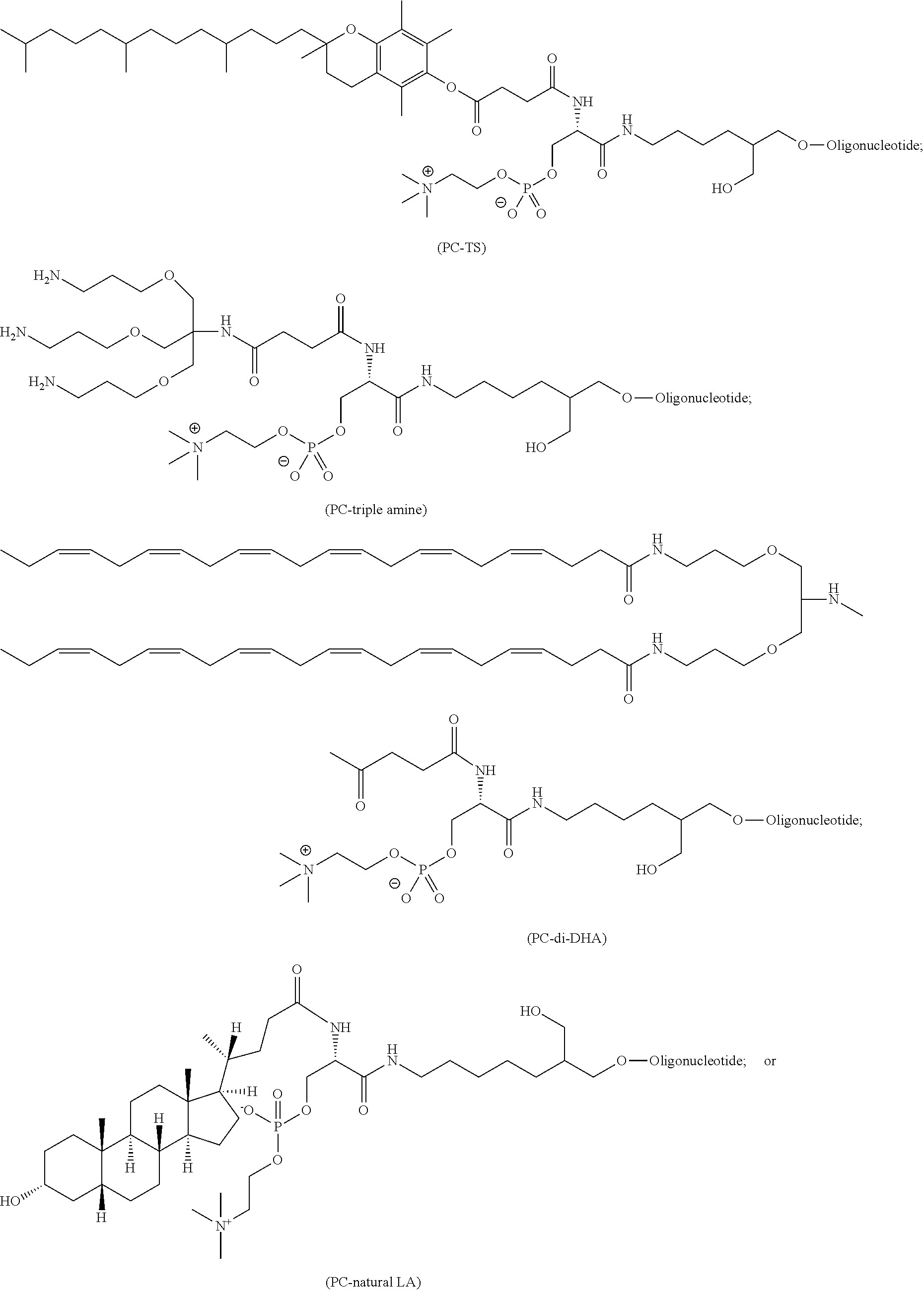

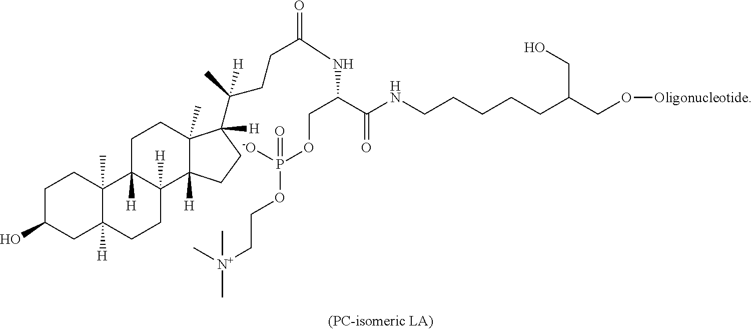

For any of the above recited structures, the term “oligonucleotide” corresponds to an oligonucleotide comprising a sequence substantially complementary to a S6K1 nucleic acid sequence. In certain embodiments, the oligonucleotide is an siRNA comprising an antisense and sense strand. In certain embodiments, the antisense strand comprises a sequence substantially complementary to a S6K1 nucleic acid sequence of any one of SEQ ID NOs: 1-6.

### Branched Oligonucleotides

The S6K1 oligonucleotides described herein may be contained in a bracnhed oligonucleotide structure. The branched oligonucleotides comprise two or more oligonucleotides linked together. The different branched oligonucleotides described herein (e.g., a branched oligonucleotide with two, three, or four oligonucleotides) enhanced eye delivery of the oligonucleotide, including eye cell-specific delivery.

In certain embodiments, the two or more oligonucleotides in the branched oligonucleotide are connected to one another by one or more moieties independently selected from a linker, a spacer and a branching point.

In certain embodiments, the linker comprises an ethylene glycol chain, an alkyl chain, a peptide, an RNA, a DNA, a phosphate, a phosphonate, a phosphoramidate, an ester, an amide, a triazole, or combinations thereof.

In certain embodiments, the branching point comprises a polyvalent organic species or derivative thereof.

In another embodiment, the branching point is an amino acid derivative. In another embodiment of the branching point is selected from the formulas of:

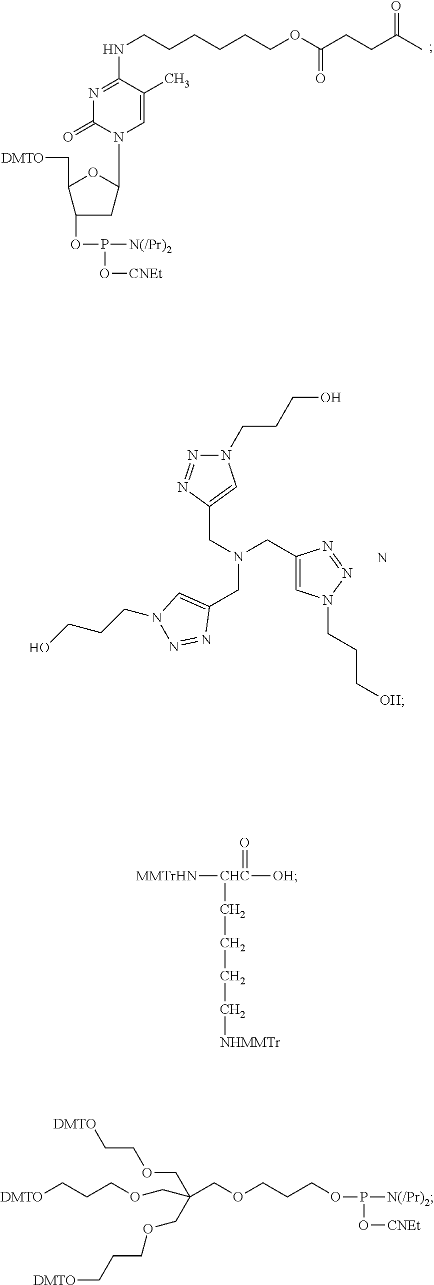

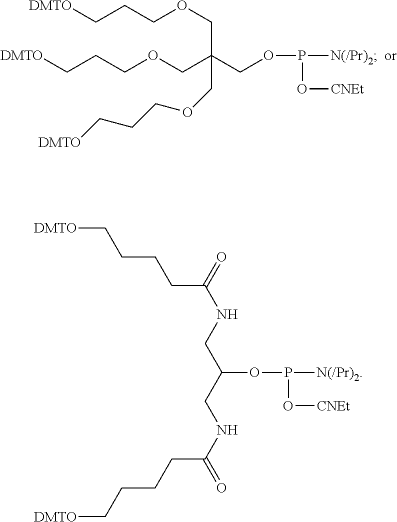

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serine, cysteine, and the like).

In certain embodiments, the spacer comprises an ethylene glycol chain, an alkyl chain, a peptide, an RNA, a DNA, a phosphate, a phosphonate, a phosphoramidate, an ester, an amide, a triazole, or a combination thereof.

In certain embodiments, the linker comprises the structure L1:

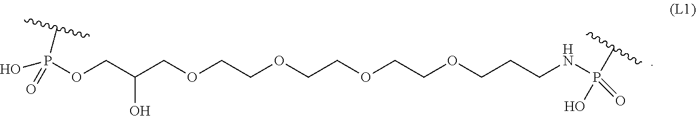 [SMILES: *P(=O)(O)NCCCOCCOCCOCCOCC(O)COP(*)(=O)O]

In certain embodiments, the linker comprises the structure L2:

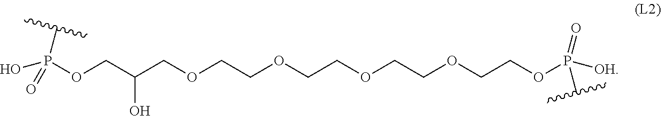 [SMILES: *P(=O)(O)OCCOCCOCCOCCOCC(O)COP(*)(=O)O]

In certain embodiments, the branched oligonucleotide consists of two oligonucleotides. In certain embodiments, the branched oligonucleotide consists of three oligonucleotides. In certain embodiments, the branched oligonucleotide consists of four oligonucleotides. In certain embodiments, the oligonucleotides are siRNA.

In certain embodiments, the branched oligonucleotide comprises the structure:

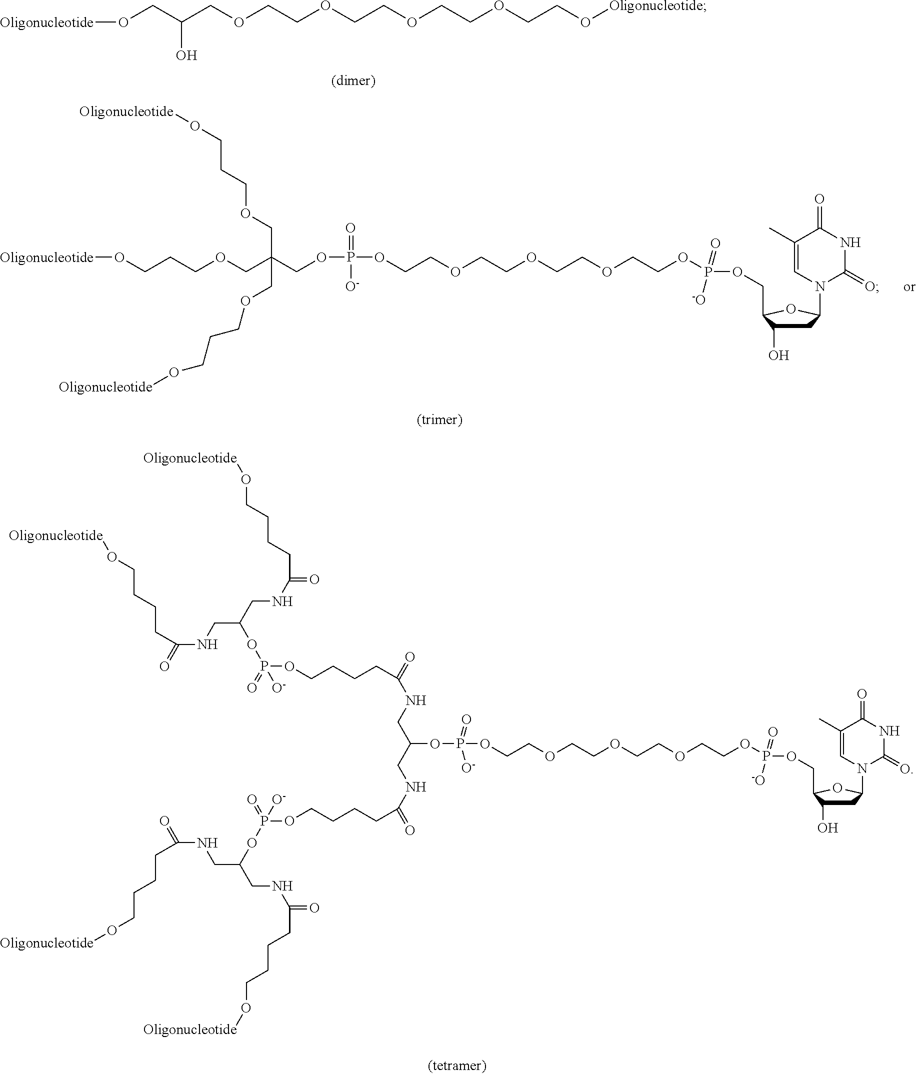

For any of the above recited structures, the term “oligonucleotide” corresponds to any of the oligonucleotides recited herein, e.g., an ASO or siRNA. In certain embodiments, the term “oligonucleotide” in the structures recited above corresponds to the sense strand of an siRNA. In certain embodiments, the oxygen immediately adjacent to the term “oligonucleotide” in the structures is linked to the 3′ end of a sense strand of an siRNA.

Branched oligonucleotides, including synthesis and methods of use, are described in greater detail in WO2017/132669, incorporated herein by reference. Further details regarding synthesis are provided in the Materials and Methods section of the Examples.

### Methods of Introducing Nucleic Acids, Vectors and Host Cells

RNA silencing agents of the disclosure may be directly introduced into the cell (e.g., an eye cell) (i.e., intracellularly); or introduced extracellularly into a cavity, interstitial space, into the circulation of an organism, introduced orally, or may be introduced by bathing a cell or organism in a solution containing the nucleic acid. Vascular or extravascular circulation, the blood or lymph system, and the cerebrospinal fluid are sites where the nucleic acid may be introduced.

The RNA silencing agents of the disclosure can be introduced using nucleic acid delivery methods known in art including injection of a solution containing the nucleic acid, bombardment by particles covered by the nucleic acid, soaking the cell or organism in a solution of the nucleic acid, or electroporation of cell membranes in the presence of the nucleic acid. Other methods known in the art for introducing nucleic acids to cells may be used, such as lipid-mediated carrier transport, chemical-mediated transport, and cationic liposome transfection such as calcium phosphate, and the like. The nucleic acid may be introduced along with other components that perform one or more of the following activities: enhance nucleic acid uptake by the cell or other-wise increase inhibition of the target gene.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus, the RNA may be introduced along with components that perform one or more of the following activities: enhance RNA uptake by the cell, inhibit annealing of single strands, stabilize the single strands, or other-wise increase inhibition of the target gene.

The cell having the target gene may be from the germ line or somatic, totipotent or pluripotent, dividing or non-dividing, parenchyma or epithelium, immortalized or transformed, or the like. The cell may be a stem cell or a differentiated cell. Cell types that are differentiated include eye cells, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands.

Depending on the particular target gene and the dose of double stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, Enzyme Linked ImmunoSorbent Assay (ELISA), Western blotting, RadiolmmunoAssay (RIA), other immunoassays, and Fluorescence Activated Cell Sorting (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present disclosure. Lower doses of injected material and longer times after administration of RNAi agent may result in inhibition in a smaller fraction of cells (e.g., at least 10%, 20%, 50%, 75%, 90%, or 95% of targeted cells). Quantization of gene expression in a cell may show similar amounts of inhibition at the level of accumulation of target mRNA or translation of target protein. As an example, the efficiency of inhibition may be determined by assessing the amount of gene product in the cell; mRNA may be detected with a hybridization probe having a nucleotide sequence outside the region used for the inhibitory double-stranded RNA, or translated polypeptide may be detected with an antibody raised against the polypeptide sequence of that region.

The RNA may be introduced in an amount which allows delivery of at least one copy per cell. Higher doses (e.g., at least 5, 10, 100, 500 or 1000 copies per cell) of material may yield more effective inhibition; lower doses may also be useful for specific applications.

In an exemplary aspect, the efficacy of an RNAi agent of the disclosure (e.g., an siRNA targeting an S6K1 target sequence) is tested for its ability to specifically degrade mutant mRNA (e.g., S6K1 mRNA and/or the production of S6K1protein) in cells, such as cells in the central nervous system. In certain embodiments, cells in the central nervous system include, but are not limited to, neurons (e.g., striatal or cortical neuronal clonal lines and/or primary neurons), glial cells, and astrocytes. Also suitable for cell-based validation assays are other readily transfectable cells, for example, HeLa cells or COS cells. Cells are transfected with human wild type or mutant cDNAs (e.g., human wild type or mutant S6K1cDNA). Standard siRNA, modified siRNA or vectors able to produce siRNA from U-looped mRNA are co-transfected. Selective reduction in target mRNA (e.g., S6K1mRNA) and/or target protein (e.g., S6K1protein) is measured. Reduction of target mRNA or protein can be compared to levels of target mRNA or protein in the absence of an RNAi agent or in the presence of an RNAi agent that does not target S6K1 mRNA. Exogenously-introduced mRNA or protein (or endogenous mRNA or protein) can be assayed for comparison purposes. When utilizing neuronal cells, which are known to be fairly resistant to standard transfection techniques, it may be desirable to introduce RNAi agents (e.g., siRNAs) by passive uptake.

### Recombinant Adeno-Associated Viruses and Vectors

In certain exemplary embodiments, recombinant adeno-associated viruses (rAAVs) and their associated vectors can be used to deliver one or more siRNAs into cells, e.g., neural cells (e.g., brain cells). AAV is able to infect many different cell types, although the infection efficiency varies based upon serotype, which is determined by the sequence of the capsid protein. Several native AAV serotypes have been identified, with serotypes 1-9 being the most commonly used for recombinant AAV. AAV-2 is the most well-studied and published serotype. The AAV-DJ system includes serotypes AAV-DJ and AAV-DJ/8. These serotypes were created through DNA shuffling of multiple AAV serotypes to produce AAV with hybrid capsids that have improved transduction efficiencies in vitro (AAV-DJ) and in vivo (AAV-DJ/8) in a variety of cells and tissues.

In certain embodiments, widespread central nervous system (CNS) delivery can be achieved by intravascular delivery of recombinant adeno-associated virus 7 (rAAV7), RAAV9 and rAAV10, or other suitable rAAVs (Zhang et al. (2011) Mol. Ther. 19(8):1440-8. doi: 10.1038/mt.2011.98. Epub 2011 May 24). rAAVs and their associated vectors are well-known in the art and are described in US Patent Applications 2014/0296486, 2010/0186103, 2008/0269149, 2006/0078542 and 2005/0220766, each of which is incorporated herein by reference in its entirety for all purposes.

rAAVs may be delivered to a subject in compositions according to any appropriate methods known in the art. An rAAV can be suspended in a physiologically compatible carrier (i.e., in a composition), and may be administered to a subject, i.e., a host animal, such as a human, mouse, rat, cat, dog, sheep, rabbit, horse, cow, goat, pig, guinea pig, hamster, chicken, turkey, a non-human primate (e.g., Macaque) or the like. In certain embodiments, a host animal is a non-human host animal.

Delivery of one or more rAAVs to a mammalian subject may be performed, for example, by intramuscular injection or by administration into the bloodstream of the mammalian subject. Administration into the bloodstream may be by injection into a vein, an artery, or any other vascular conduit. In certain embodiments, one or more rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer virions into the vasculature of an isolated limb to potentially enhance transduction into muscle cells or tissue. Moreover, in certain instances, it may be desirable to deliver virions to the central nervous system (CNS) of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

The compositions of the disclosure may comprise an rAAV alone, or in combination with one or more other viruses (e.g., a second rAAV encoding having one or more different transgenes). In certain embodiments, a composition comprises 1, 2, 3, 4, 5, 6, 7, 8, 9, 10 or more different rAAVs each having one or more different transgenes.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of one or more rAAVs is generally in the range of from about 1 ml to about 100 ml of solution containing from about 10⁹ to 10¹⁶ genome copies. In some cases, a dosage between about 10¹¹ to 10¹² rAAV genome copies is appropriate. In certain embodiments, 10¹² rAAV genome copies is effective to target heart, liver, and pancreas tissues. In some cases, stable transgenic animals are produced by multiple doses of an rAAV.

In some embodiments, rAAV compositions are formulated to reduce aggregation of AAV particles in the composition, particularly where high rAAV concentrations are present (e.g., about 10 13 genome copies/mL or more). Methods for reducing aggregation of rAAVs are well known in the art and, include, for example, addition of surfactants, pH adjustment, salt concentration adjustment, etc. (See, e.g., Wright et al. (2005) Molecular Therapy 12:171-178, the contents of which are incorporated herein by reference.)

“Recombinant AAV (rAAV) vectors” comprise, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule (e.g., siRNA) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are usually about 145 basepairs in length. In certain embodiments, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including mammalian AAV types described further herein.

Methods of Treatment

In one aspect, the present disclosure provides for both prophylactic and therapeutic methods of treating a subject at risk of (or susceptible to) developing a disease associated with S6K1 expression. In one embodiment, the disease is cancer. In one embodiment, the disease is obesity. In one embodiment, the disease is diabetes. In one embodiment, the disease is an eye disease. In one embodiment, the eye disease is selected from the group consisting of age-related macular degeneration, diabetic retinopathy, central cataract, normal-tension glaucoma, macular edema, and glaucoma.

“Treatment,” or “treating,” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the disclosure provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the disclosure pertains to methods treating subjects therapeutically, i.e., alter onset of symptoms of the disease or disorder. In an exemplary embodiment, the modulatory method of the disclosure involves contacting a CNS cell expressing S6K1 with a therapeutic agent (e.g., a RNAi agent or vector or transgene encoding same) that is specific for a target sequence within the gene (e.g., S6K1target sequences of Table 1), such that sequence specific interference with the gene is achieved. These methods can be performed in vitro (e.g., by culturing the cell with the agent) or, alternatively, in vivo (e.g., by administering the agent to a subject).

### Pharmaceutical Compositions and Methods of Administration

The disclosure pertains to uses of the above-described agents for prophylactic and/or therapeutic treatments as described infra. Accordingly, the modulators (e.g., RNAi agents) of the present disclosure can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intravitreal, intradermal, subcutaneous, intraperitoneal, intramuscular, oral (e.g., inhalation), transdermal (topical), and transmucosal administration. In certain exemplary embodiments, the pharmaceutical composition of the disclosure is administered intravenously and is capable of crossing the blood brain barrier to enter the central nervous system In certain exemplary embodiments, a pharmaceutical composition of the disclosure is delivered to the cerebrospinal fluid (CSF) by a route of administration that includes, but is not limited to, intrastriatal (IS) administration, intracerebroventricular (ICV) administration and intrathecal (IT) administration (e.g., via a pump, an infusion or the like).

The nucleic acid molecules of the disclosure can be inserted into expression constructs, e.g., viral vectors, retroviral vectors, expression cassettes, or plasmid viral vectors, e.g., using methods known in the art, including but not limited to those described in Xia et al., (2002), Supra. Expression constructs can be delivered to a subject by, for example, inhalation, orally, intravenous injection, local administration (see U.S. Pat. No. 5,328,470) or by stereotactic injection (see e.g., Chen et al. (1994), Proc. Natl. Acad. Sci. USA, 91, 3054-3057). The pharmaceutical preparation of the delivery vector can include the vector in an acceptable diluent, or can comprise a slow-release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The nucleic acid molecules of the disclosure can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al. (2002), Science, 296, 550-553; Lee et al, (2002). supra; Miyagishi and Taira (2002), Nature Biotechnol., 20, 497-500; Paddison et al. (2002), supra; Paul (2002), supra; Sui (2002) supra; Yu et al. (2002), supra.

The expression constructs may be any construct suitable for use in the appropriate expression system and include, but are not limited to retroviral vectors, linear expression cassettes, plasmids and viral or virally-derived vectors, as known in the art. Such expression constructs may include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct, Tuschl (2002), Supra.

In certain embodiments, a composition that includes a compound of the disclosure can be delivered to the nervous system of a subject by a variety of routes. Exemplary routes include intrathecal, parenchymal (e.g., in the brain), nasal, and ocular delivery. The composition can also be delivered systemically, e.g., by intravenous, subcutaneous or intramuscular injection. One route of delivery is directly to the brain, e.g., into the ventricles or the hypothalamus of the brain, or into the lateral or dorsal areas of the brain. The compounds for neural cell delivery can be incorporated into pharmaceutical compositions suitable for administration.

For example, compositions can include one or more species of a compound of the disclosure and a pharmaceutically acceptable carrier. The pharmaceutical compositions of the present disclosure may be administered in a number of ways depending upon whether local or systemic treatment is desired and upon the area to be treated. Administration may be topical (including ophthalmic, intranasal, transdermal), oral or parenteral. Parenteral administration includes intravenous drip, subcutaneous, intraperitoneal or intramuscular injection, intrathecal, or intraventricular (e.g., intracerebroventricular) administration. In certain exemplary embodiments, an RNA silencing agent of the disclosure is delivered across the Blood-Brain-Barrier (BBB) suing a variety of suitable compositions and methods described herein.

The route of delivery can be dependent on the disorder of the patient. For example, a subject diagnosed with a neurodegenerative disease can be administered an anti-S6K1 compounds of the disclosure directly into the brain (e.g., into the globus pallidus or the corpus striatum of the basal ganglia, and near the medium spiny neurons of the corpus striatum). In addition to a compound of the disclosure, a patient can be administered a second therapy, e.g., a palliative therapy and/or disease-specific therapy. The secondary therapy can be, for example, symptomatic (e.g., for alleviating symptoms), neuroprotective (e.g., for slowing or halting disease progression), or restorative (e.g., for reversing the disease process). Other therapies can include psychotherapy, physiotherapy, speech therapy, communicative and memory aids, social support services, and dietary advice.

A compound of the disclosure can be delivered to neural cells of the brain. In certain embodiments, the compounds of the disclosure may be delivered to the brain without direct administration to the central nervous system, i.e., the compounds may be delivered intravenously and cross the blood brain barrier to enter the brain. Delivery methods that do not require passage of the composition across the blood-brain barrier can be utilized. For example, a pharmaceutical composition containing a compound of the disclosure can be delivered to the patient by injection directly into the area containing the disease-affected cells. For example, the pharmaceutical composition can be delivered by injection directly into the brain. The injection can be by stereotactic injection into a particular region of the brain (e.g., the substantia nigra, cortex, hippocampus, striatum, or globus pallidus). The compound can be delivered into multiple regions of the central nervous system (e.g., into multiple regions of the brain, and/or into the spinal cord). The compound can be delivered into diffuse regions of the brain (e.g., diffuse delivery to the cortex of the brain).

In one embodiment, the compound can be delivered by way of a cannula or other delivery device having one end implanted in a tissue, e.g., the brain, e.g., the substantia nigra, cortex, hippocampus, striatum or globus pallidus of the brain. The cannula can be connected to a reservoir containing the compound. The flow or delivery can be mediated by a pump, e.g., an osmotic pump or minipump, such as an Alzet pump (Durect, Cupertino, CA). In one embodiment, a pump and reservoir are implanted in an area distant from the tissue, e.g., in the abdomen, and delivery is effected by a conduit leading from the pump or reservoir to the site of release. Devices for delivery to the brain are described, for example, in U.S. Pat. Nos. 6,093,180, and 5,814,014.

### EXAMPLES

### Materials and Methods

### Synthesis of Lipid Functionalized Solid Supports

Non-phosphocholine (PC) lipid moieties (except α-tocopheryl succinate) were directly attached via a peptide bond to a controlled pore glass (CPG) functionalized by a C7 linker, as described previously (Nikan M, Osborn MF, Coles A H, et al. Docosahexaenoic acid conjugation enhances distribution and safety of siRNA upon local administration in mouse brain. Mol. Ther. Nucleic Acids. 2016; 5:e344). To synthesize PC derivatives, amino C7 CPG was first functionalized with phosphocholine (Nikan M, Osborn MF, Coles AH, et al. Synthesis and evaluation of parenchymal retention and efficacy of a metabolically stable O-Phosphocholine-N-docosahexaenoyl-l-serine siRNA conjugate in mouse brain. Bioconjug. Chem. 2017; 28:758-1766). Briefly, Fmoc-L-serine tert-butyl (TCI America) was phosphitylated using 2′-cyanoethyl-N,N-diisopropylchlorophosphoramidite (ChemGenes). The resulting phosphoramidite was coupled to choline p-toluenesulfonate (Alfa Aesar) using 5-(ethylthio)-1H-tetrazole (ETT) as an activator. The phosphine ester was then oxidized, and the carboxylic acid and phosphate ester groups were deprotected (i.e., tert-butyl and cyanoethyl groups removed). The resulting intermediate was attached to the amino C7 CPG via a peptide bond to form phosphocholine-functionalized CPG. The Fmoc group was removed, and the selected lipid moiety was attached via a peptide bond to the CPG. All lipid-functionalized solid supports were obtained with a loading of 55 Nmol/g.

Synthesis of α-tocopheryl Succinate-Conjugated Oligonucleotides

α-tocopheryl succinate was attached to the amino group at the 3′end of the purified oligocnucleotide synthesized on amino C7 CPG or phosphocholine-functionalized amino C7 CPG. N-hydroxysuccinimide α-tocopheryl succinate and purified oligonucleotides were combined in a solution of 0.1 M sodium bicarbonate, 20% (v/v) dimethylformamide and incubated overnight at room temperature. One-tenth volume of 3M sodium acetate (pH 5.2) was added to obtain a final concentration of 0.3M sodium acetate. Three volumes 95% (v/v) ethanol were added, and the mixture was vortexed and then placed for 1 h at −80° C. The solution was pelleted by centrifugation for 30 min at 5200×g. The pellet containing the lipid-conjugated siRNA sense strand was dissolved in water, purified, and desalted as described below.

### Oligonucleotide Synthesis

Oligonucleotides were synthesized by phosphoramidite solid-phase synthesis on a Dr Oligo 48 (Biolytic, Fremont, CA) or MerMadel2 (Biosearch Technologies, Novato, CA), using 2′-F or 2′-O-Me modified phosphoramidites with standard protecting groups. 5′-(E)-Vinyl tetra phosphonate (pivaloyloxymethyl) 2′-O-methyl-uridine 3′-CE phosphoramidite (VP) for in vivo unconjugated oligonucleotides was purchased from Hongene Biotech, USA, Quasar 570 CE phosphoramidite (Cy3) was purchased from GenePharma, Shanghai, China. Bis-cyanoethyl-N,N-diisopropyl CED phosphoramidite (5′P) for in vitro unconjugated oligonucleotides and all other phosphoramidites used were purchased from ChemGenes, Wilmington, MA. Phosphoramidites were prepared at 0.1 M in anhydrous acetonitrile (ACN), except for 2′-O-methyl-uridine phosphoramidite dissolved in anhydrous ACN containing 15% dimethylformamide. 5-(Benzylthio)-1H-tetrazole (BTT) was used as the activator at 0.25 M, and the coupling time for all phosphoramidites was 4 min, using 10eq. Detritylations were performed using 3% trichloroacetic acid in dichloromethane. Capping reagents used were CAP A (20% n-methylimidazole in ACN) and CAP B (20% acetic anhydride and 30% 2,6-lutidine in ACN). Reagents for capping and detritylation were purchased from AIC, Framingham, MA. Phosphite oxidation to convert to phosphate or phosphorothioate was performed with 0.05 M iodine in pyridine-H20 (9:1, v/v) or 0.1 M solution of 3-[(dimethylaminomethylene)amino]-3H-1,2,4-dithiazole-5-thione (DDTT) in pyridine (ChemGenes) for 4 min. Unconjugated oligonucleotides were synthesized on 500Å long-chain alkyl amine (LCAA) controlled pore glass (CPG) functionalized with Unylinker terminus (ChemGenes). Cholesterol conjugated oligonucleotides were synthesized on a 500Å LCAA-CPG support, where the cholesterol moiety is bound to tetra-ethylenglycol through a succinate linker (ChemGenes, Wilmington, MA). Lipid conjugated oligonucleotides were synthesized on modified solid support (synthesis described above). Divalent oligonucleotides (Dimer) were synthesized on modified solid support, synthesis described previously (Alterman JF, Godinho BMDC, Hassler MR, et al. A divalent siRNA chemical scaffold for potent and sustained modulation of gene expression throughout the central nervous system. Nat Biotechnol 37, 884-894 (2019)).

### Branched Oligonucleotides Synthesis

Synthesis of branched oligonucleotides was performed by phosphoramidite solid-phase, on an AKTA Oligoplilot 10 (Cytiva, Marlborough, MA) with the parameters described above, or otherwise specified here. Trimer and Tetramer branched oligonucleotides were prepared using commercial trebler and doubler phosphoramidites respectively purchased from Glen Research, Sterling, VA. Trimer linker was produced in two steps as follows; on a 1000Å Thymidine 3′-LCAA-CPG (ChemGenes), DMT-tetraethyloxy-Glycol CED phosphoramidite (ChemGenes) was coupled first for 8 min, using 10 eq, followed by the coupling of the trebler phosphoramidite for 8 min, using 10 eq. Trivalent oligonucleoides were grown afterwards on this linker using 30 eq. Tetramer linker was produced in three steps as follows; first, on a 1000Å Thymidine 3′-LCAA-CPG, DMT-tetraethyloxy-Glycol CED phosphoramidite was coupled for 8 min, using 10 eq, second, the coupling of doubler phosphoramidite for 8 min, using 10 eq, and third, a subsequent coupling of doubler phosphoramidite for 8 min, using 20 eq was performed. Tetravalent oligonucleotides were grown afterwards using 40 eq.

### Deprotection and Purification of Oligonucleotides for Screening of Sequences

Prior to the deprotection, synthesis columns containing oligonucleotides were treated with 10% diethylamine (DEA) in ACN to deprotect cyanoethyl groups. In synthesis columns, both unconjugated and cholesterol conjugated oligonucleotides on solid support were then deprotected with methylamine gas (Airgas) for an hour at room temperature. Deprotected oligonucleotides released from the solid support were precipitated on the support by passing solution of (i) a mixture of 0.1 M sodium acetate in 85% ethanol and then (ii) 85% ethanol to the synthesis column. The excess ethanol on solid support was dried by air flow and the oligonucleotides were flushed out by passing water through the column. This procedure renders pure oligonucleotides used for in vitro experiments.

### Deprotection and Purification of Oligonucleotides for In Vivo Experiments

Prior to the deprotection, synthesis columns containing oligonucleotides were treated with 10% diethylamine (DEA) in ACN to deprotect cyanoethyl groups. Cy3 labeled and lipid conjugated oligonucleotides were cleaved and deprotected in 28-30% ammonium hydroxide, 40% aq. methylamine (1:1, v/v) (AMA) for 2 h at room temperature. Cy3 labeled and non-labeled unconjugated, divalent, trivalent, and tetravalent oligonucleotides were cleaved and deprotected by AMA treatment at 45° C. for 2 h. The VP containing oligonucleotides did not have a pretreatment with DEA post-synthesis and were cleaved, and deprotected as described previously (O'Shea J, Theile C S, Das R, et al, An efficient deprotection method for 5′[O,O-bis(pivaloyloxymethyl)]-(E)-vinylphosphonate containing oligonucleotides. Tetrahedron 74, 6182-6186 (2018)). Briefly, CPG with VP-oligonucleotides was treated with a solution of 3% DEA in 28-30% ammonium hydroxide at 35° C. for 20 hours.

All solutions containing cleaved oligonucleotides were filtered to remove the CPG and dried under vacuum. The resulting pellets were re-suspended in 5% ACN in water. Purifications were performed on an Agilent 1290 Infinity II HPLC system. VP and non-labeled unconjugated, divalent, trivalent and tetravalent oligonucleotides were purified using a custom 25×150mm column packed with Source 15Q anion exchange resin (Cytiva, Marlborough, MA); running conditions: eluent A, 10 mM Tris-HC1 buffer (pH 9) in 7.5% ACN in water; eluent B, 1 M sodium perchlorate in 10 mM Tris-HC buffer (pH 9) in 7.5% ACN in water; linear gradient, 12 to 35% B in 40 min at 50° C. Lipid conjugated and Cy3 labeled oligonucleotides were purified using a 21.2×150mm PRP-C18 column (Hamilton Co, Reno, NV); running conditions: eluent A, 50 mM sodium acetate (pH 6) in 5% ACN in water; eluent B, 100% ACN; linear gradient, 15 to 60% B in 40 min at 60° C. Flow was 40mL/min in both methods and peaks were monitored at 260 nm for non-labeled oligonucleotides and 550 nm for labeled oligonucleotides. A separate column was used for Cy3 labeled oligonucleotides to avoid cross-contamination. Fractions were analyzed by liquid chromatography mass spectrometry (LC-MS), pure fractions combined and dried under vacuum. Oligonucleotides were re-suspended in 5% ACN and desalted by size exclusion on a 25×250 mm custom column packed with Sephadex G-25 media (Cytiva, Marlborough, MA), using isocratic method with HPLC grade water (Honeywell Chemicals, Charlotte, NC) and finally oligonucleotides were lyophilized.

### LC-MS Analysis of Oligonucleotides

The identity of oligonucleotides was verified by LC-MS analysis on an Agilent 6530 accurate mass Q-TOF using the following conditions: buffer A: 100 mM 1,1,1,3,3,3-hexafluoroisopropanol (HFIP) and 9 mM triethylamine (TEA) in LC-MS grade water; buffer B:100 mM HFIP and 9 mM TEA in LC-MS grade methanol; column, Agilent AdvanceBio oligonucleotides C18; linear gradient O-40% B 5 min (Unconjugated, divalent, trivalent and tetravalent oligonucleotides); linear gradient 50-100% B 5 min (Lipid conjugated and Cy3 labeled oligonucleotides); temperature, 60° C.; flow rate, 0.85 ml/min. LC peaks were monitored at 260 nm and for labeled oligonucleotides at 550 nm. MS parameters: Source, electrospray ionization; ion polarity, negative mode; range, 100-3,200 m/z; scan rate, 2 spectra/s; capillary voltage, 4,000; fragmentor, 200 V; and gas temperature: 325° C.

### In Vivo Experiments

Intravitreal injections into adult mice were performed as previously described (Venkatesh A, Ma S, Langellotto F, et al. Retinal gene delivery by rAAV and DNA electroporation. Curr Protoc Microbio: 2013; Chapter 14:Unit 14D 14.). Injections were performed with glass needles (Clunbury Scientific LLC; Cat no. B100-58-50) using the FemtoJet from Eppendorf with a constant pressure and injection time of 300 psi and 1.5 s, respectively, to deliver ˜2 μL of fluid into the vitreous. All concentrations were adjusted to use a 2 μL injection volume for the desired amount of siRNA. Intravitreal injections into adult pigs used an Insulin injection needle to inject 100 μL of siRNA ˜2-3mm from the temporal limbus into the vitreous. Anesthesia and euthanasia of pigs was performed by animal medicine according to standard procedures. Cornea was treated with proparacaine and ophthalmic Betadine before injection of the siRNA. After injection eyes were rinsed with saline eye wash solution. Enucleated pig and mouse eyes were processed as described (Venkatesh A, Ma S, Langellotto F, et al. Retinal gene delivery by rAAV and DNA electroporation. Curr Protoc Microbio: 2013;Chapter 14:Unit 14D 14.).

### Example 1: Delivery of siRNA for Eye Diseases

An initial screen of different siRNA, all targeting Htt gene with the same sequence, was conducted in order to study their distribution and efficiency on knockdown in the eye.

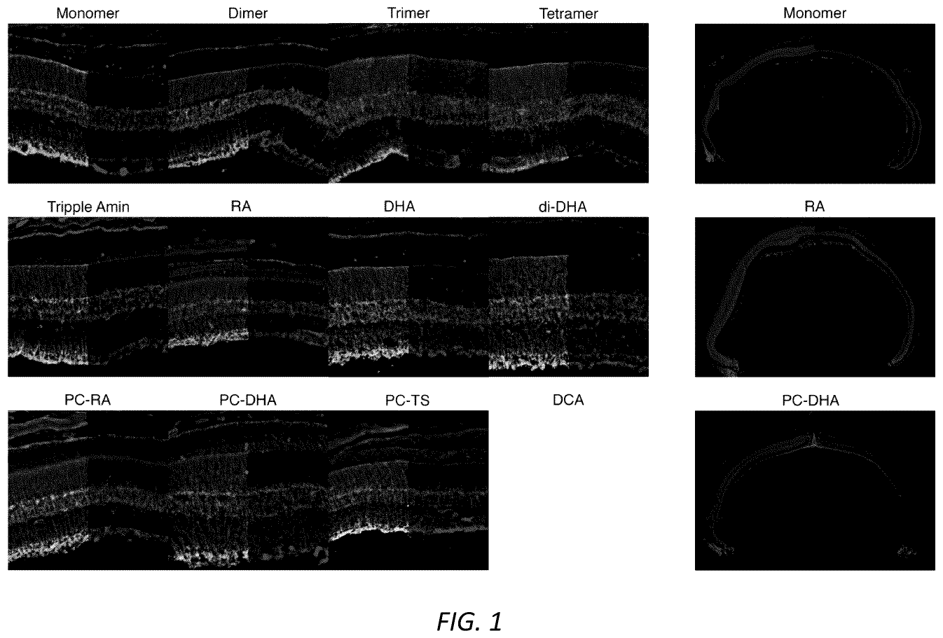

The cellular distribution is difficult to ascertain from the Cy3 label alone. Therefore, a lower dose of the siRNA compounds (0.1 nanomole) was injected (siRNAs labeled with Cy3 are shown in red in FIG. 1). 3 days later, the tissue was dissociated, Cy3 positive cells were FACS sorted, and cell type specific antibodies were used to determine which cell types were enriched by which siRNA modification.

FIG. 1 shows all siRNAs are labeled with Cy3 and in red, meanwhile glutamine synthetase (GS) expression, which is specific to Müller glia cells, is shown in green, and nuclear DAPI is shown in blue. All siRNA can be seen across the entire retinal cross section with slightly different cellular distribution. The right of FIG. 1 displays one example per group showing entire retinal cross section with distribution across the entire retina: half of the section shows nuclear DAPI, GS and the siRNA, the other half only the siRNA.

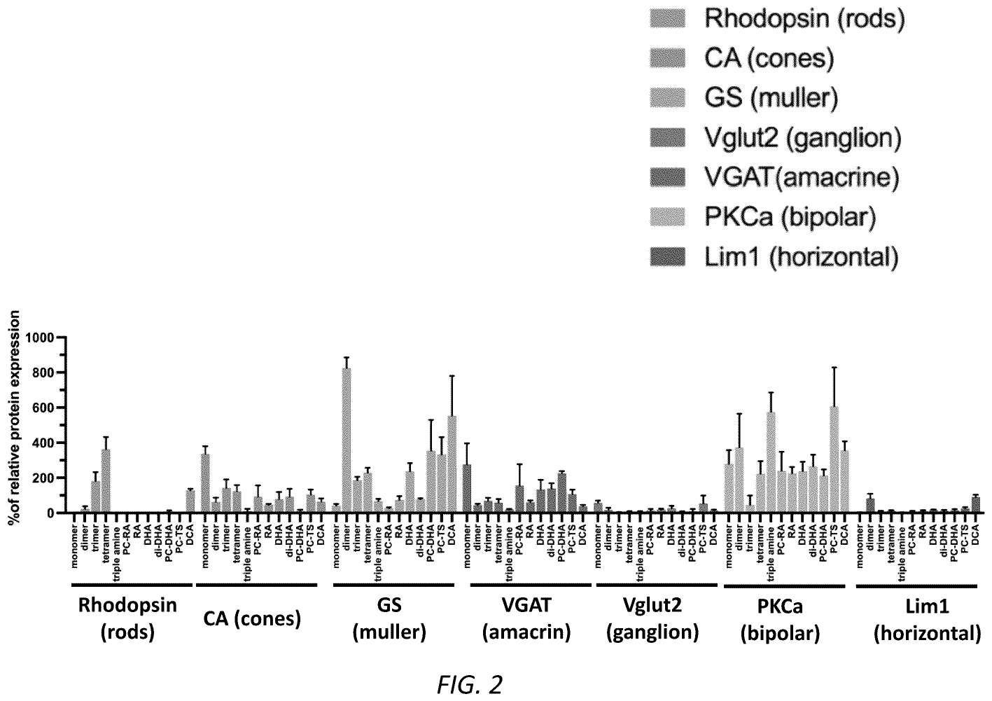

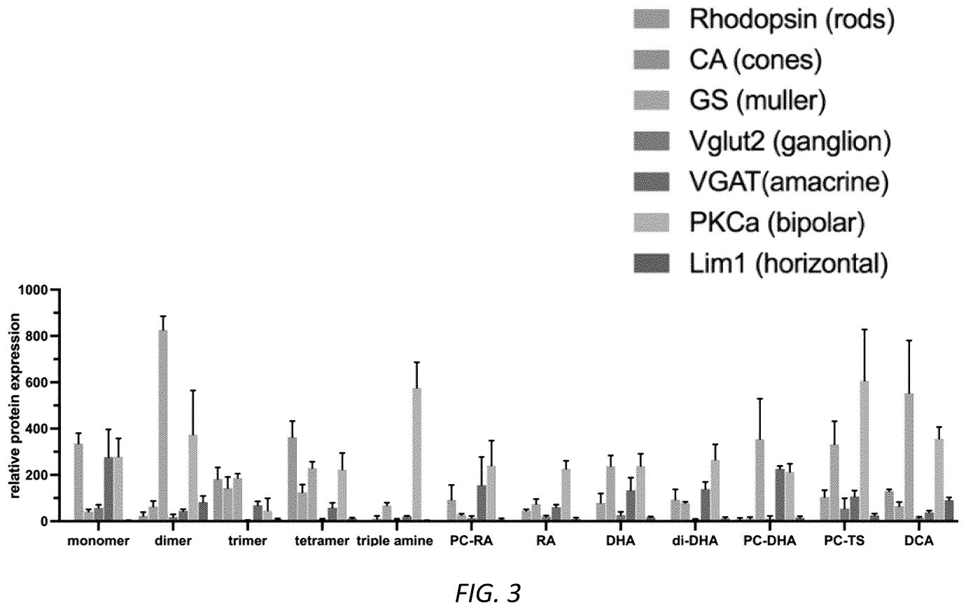

The overall goal was A) to determine the cellular distribution and B) to identify the modifications that allow for best cell entry into cone and rod photoreceptors and Müller glia cells, as these three cell types are the most important ones to target for many retinal diseases. FIGS. 2 and 3 show the outcome of the enrichment of siRNA in different retinal cell types arranged by cell type (FIG. 2) and by modification (FIG. 3). The findings suggest that the Monomer configuration is good for cone photoreceptors, the tetramer is good for rod photoreceptors and the dimer is best for Müller glia cells. Other good Müller glia cell compounds are PC-TS, PC-DHA and DCA.

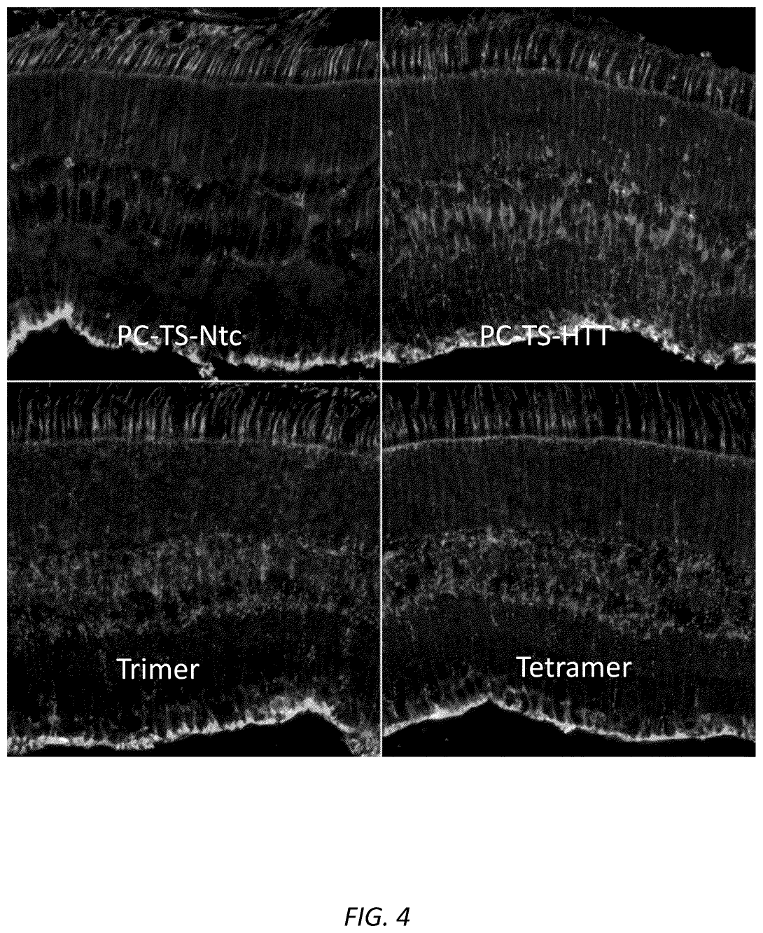

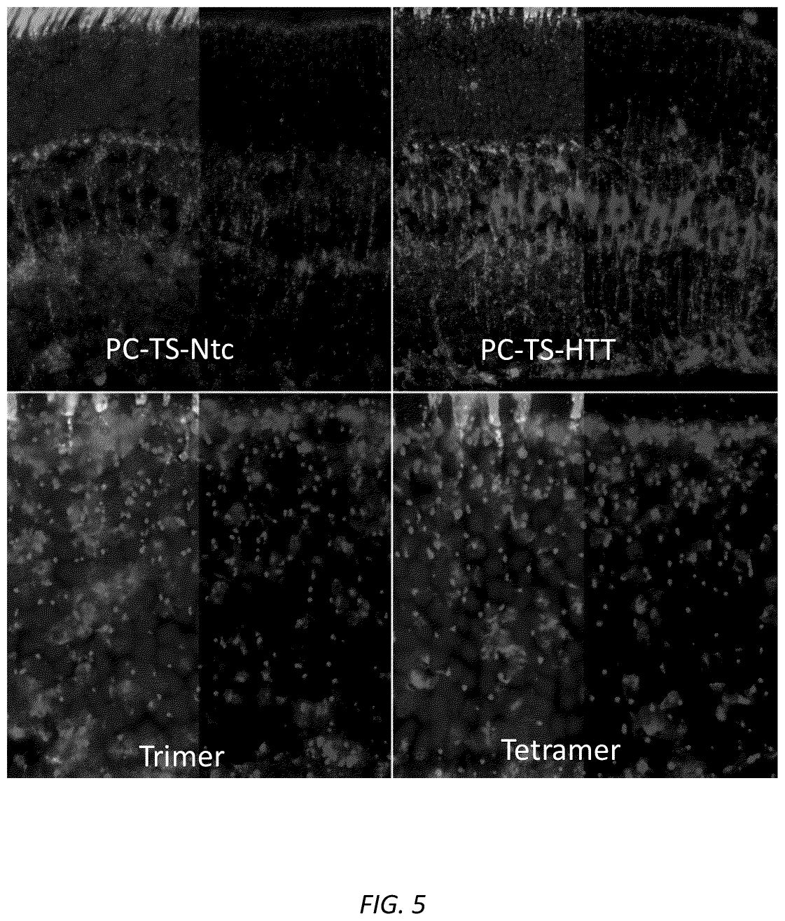

Repeat injections and higher magnifications of a subset of these compounds show that PC-TS accumulates well in Müller Glia cells and the trimer and tetramer accumulates well in rod photoreceptors (FIGS. 4 and 5).

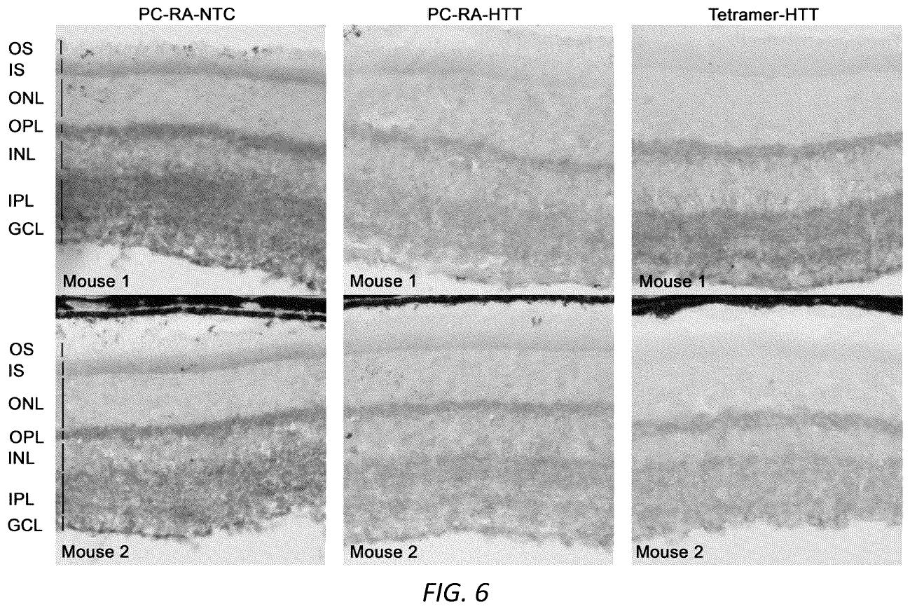

To determine if the tetramer is able to knockdown HTT protein better in photoreceptors than other configurations, an antibody staining was performed against the HTT protein two weeks after intravitreal injection of 0.3 nanomoles of the siRNAs shown in FIG. 6. First column in FIG. 6 shows antibody staining in control mice injected with the NTC-siRNA. HTT protein has a pan-retinal expression with particular enrichment in the photoreceptor inner segments (IS), in the outer plexiform layer (OPL) where photoreceptors make synaptic connections with the bipolar cells and the horizontal cells, an intermediate enrichment in the inner nuclear layer (INL), where bipolar cell, amacrine cell, horizontal cell and Mueller glia cell bodies reside, and a strong enrichment in the inner plexiform layer (IPL) where synaptic connections of amacrine, bipolar and ganglion cells reside. Second column, expression of HTT protein after knockdown with PC-RA-Htt siRNA. This siRNA tends to accumulate preferentially in bipolar and amacrine cells, as shown in FIGS. 2 & 3. Thus, expression in the OPL and IPL are reduced more efficiently with this siRNA. Third column shows expression of HTT protein in 2 different mice each injected with ˜0.3 nanomoles of the Htt-siRNA after knockdown with Tetramer-Htt siRNA. This configuration tends to accumulate efficiently in rod photoreceptors and bipolar cells. Consistent with that expression in photoreceptor ISs is reduced much more than with the PC-RA-Htt siRNA.

Quantification by Western Blotting of total HTT protein remaining from total retinal extracts was also performed two weeks after injection with the Htt-siRNA using the same experimental setting done is FIG. 6 using different mice from the same injected batch. Note that the knockdown is at about 50% relative expression at 2 weeks post injection when quantifying total HTT protein. The two configurations targeted the different cell population of the retina at different efficiencies. Nonetheless, the overall knockdown is similar due to the ubiquitous expression of HTT in the retina.

### Example 2: Assessment of Htt mRNA Knockdown by bDNA Assays

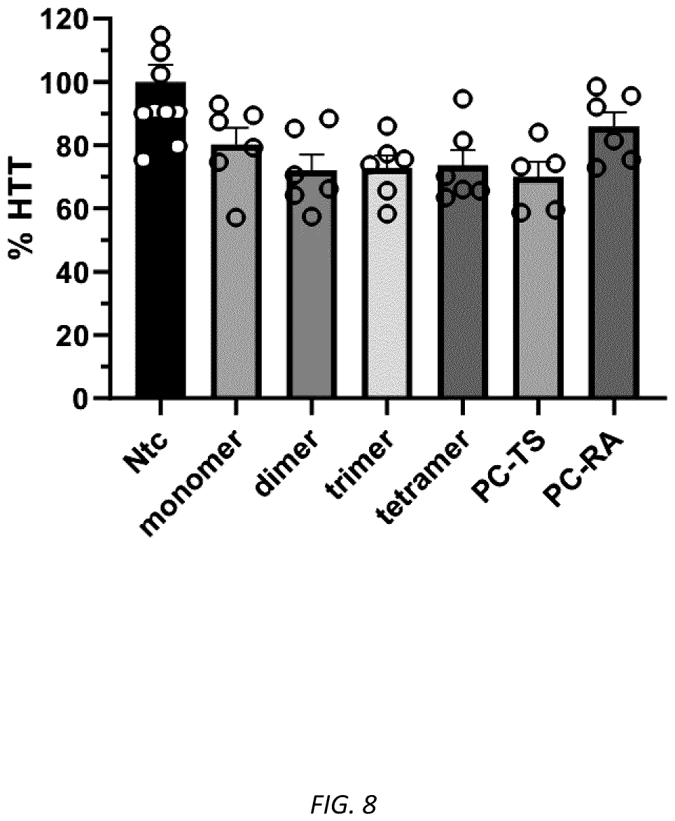

bDNA assay was used to quantify total Htt mRNA levels two weeks after injection with the Htt-siRNA using 0.1 nanomole per injection of stated siRNA modification. 0.1 nanomole resulted in about 20%-30% knockdown at 2 weeks post injection when quantifying Htt mRNA levels as shown in FIG. 8.

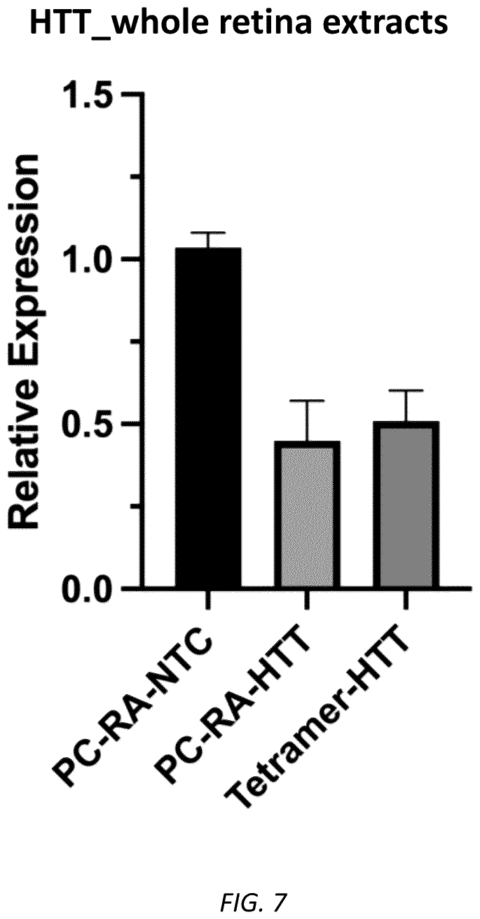

bDNA assay was also used to quantify total Htt mRNA levels 3 days after injection with the Htt-siRNA using 0.3 nanomoles per injection of stated siRNA modification. Note, 0.3 nanomoles results in about 30%-60% knockdown at 3 days post injection when quantifying Htt mRNA levels. Also, PC-RA shows similar percentage knockdown when compared to total protein measurements at 2 weeks post injection (FIG. 7: 60% knockdown), indicating that both quantifications methods are similar for the Htt gene in the retina and that there is a direct correlation between mRNA and protein levels for this gene (each dot represents 1 retina).

### Example 3: Long-Term Assessment of Htt mRNA Knockdown by bDNA Assays

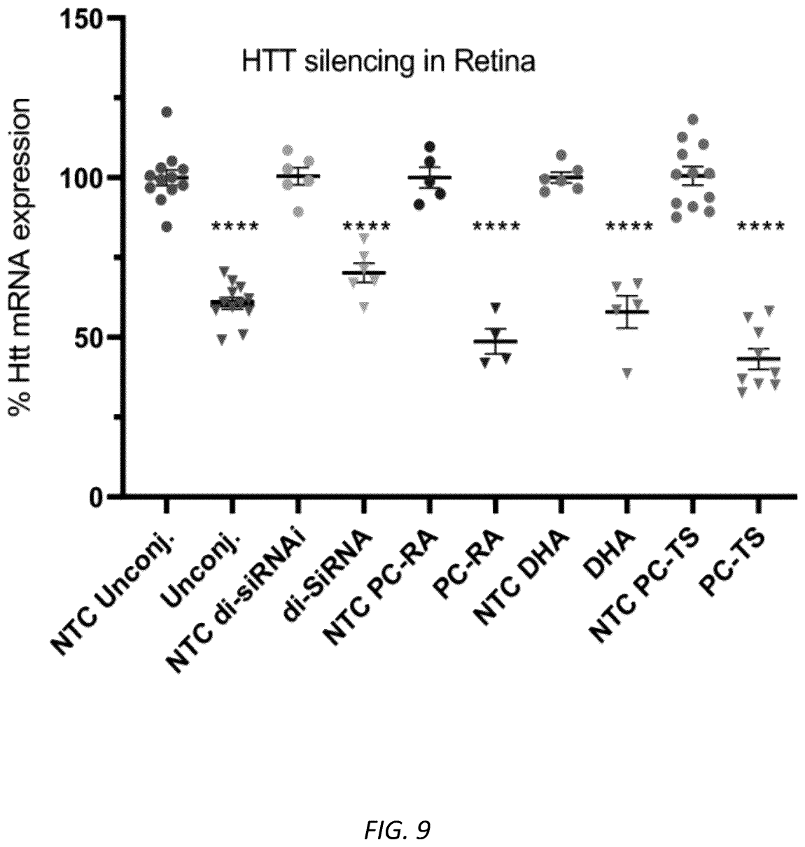

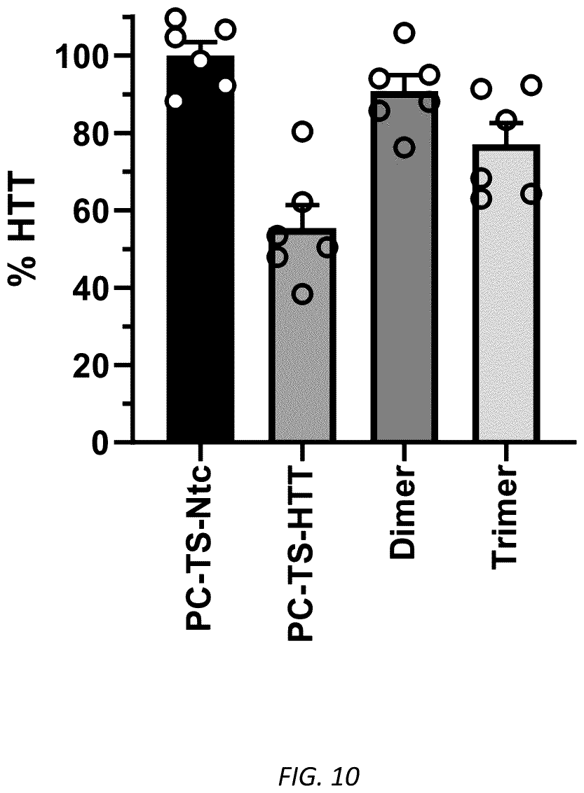

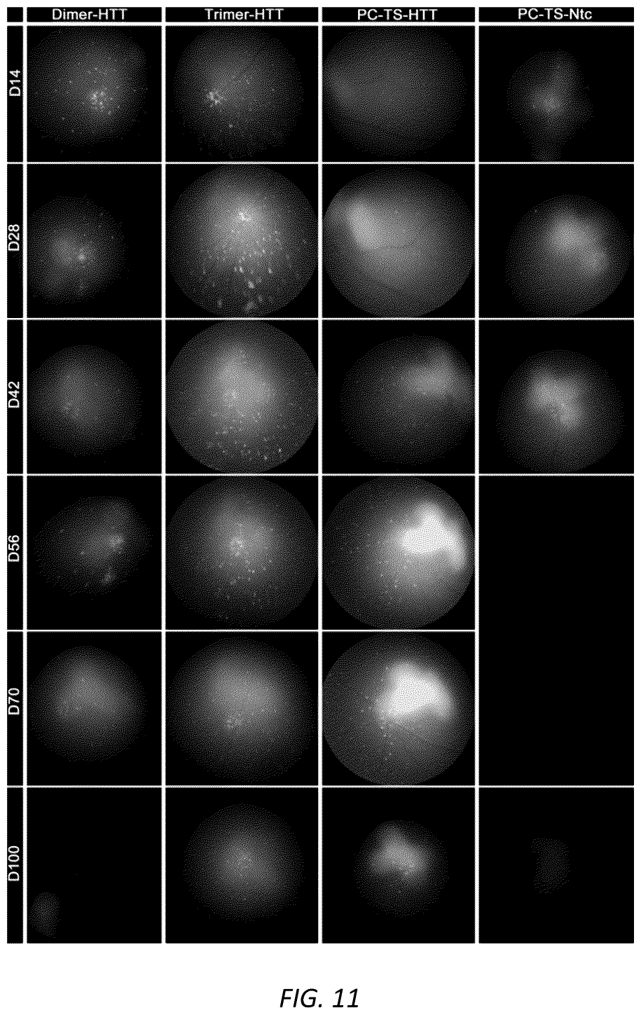

FIG. 10 shows the results of the quantification by bDNA assay performed to quantify total Htt mRNA levels 100 days after injection with the Htt-siRNA using 0.3 nanomoles per injection of stated siRNA modification. Note, compared to FIG. 9 the knockdown effect changed only by ˜10% over a time window of ˜100 days (60% to 50% knockdown), indicating that the knockdown is very stable (each dot represents 1 retina in FIG. 10). FIG. 11 shows representative fundus images over time of eyes injected with the Cy3 labeled siRNAs with modifications as indicated on the figure. Exposure of fluorescence signal is the same for all 4 siRNA at any given time point, but not over time. FIG. 11 complements FIG. 10 showing the fundus images of the mice used in FIG. 10. All mice were injected with 0.3 nanomoles of siRNA intravitreally.

### Example 4: Dose Escalation Study of HTT Knockdown in Mouse with Tetramer Configuration

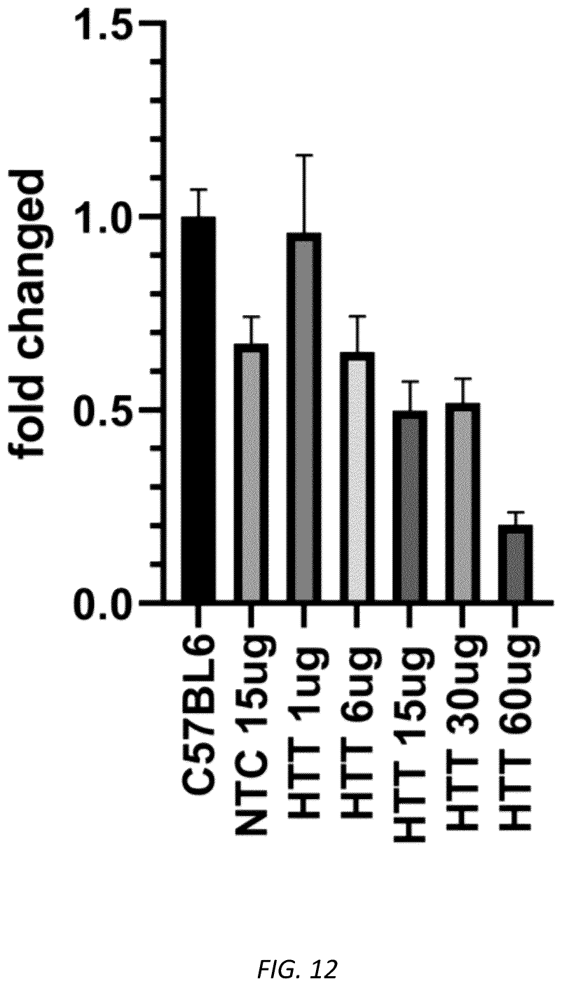

FIG. 12 shows the results of the dose escalation study of for HTT-knockdown in retina. Mice were injected with amounts indicated in the FIG. 1-60 of Cy3 labeled Tetramer with Htt-siRNA) in a total volume of 2 microliter. Five mice were injected per amount of siRNA. Tissue was harvested at 2 weeks post-injection to perform quantification by western blotting of remaining HTT protein in retina. Injections with 15-30 microgram correspond roughly to the same knockdown seen with ˜0.3 nanomoles in previous experiments.

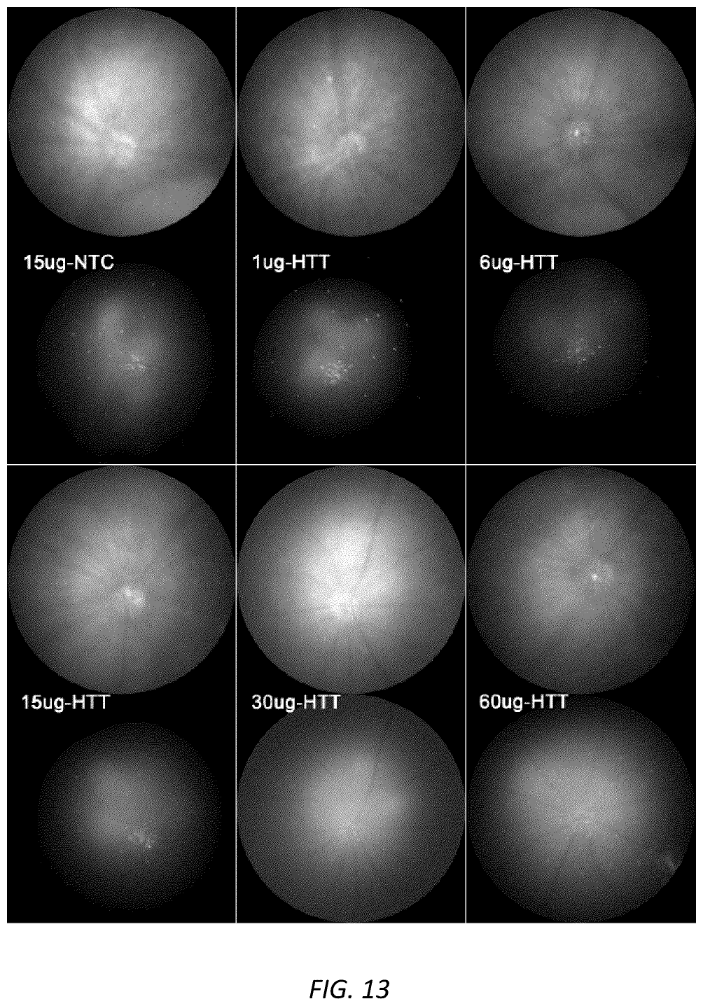

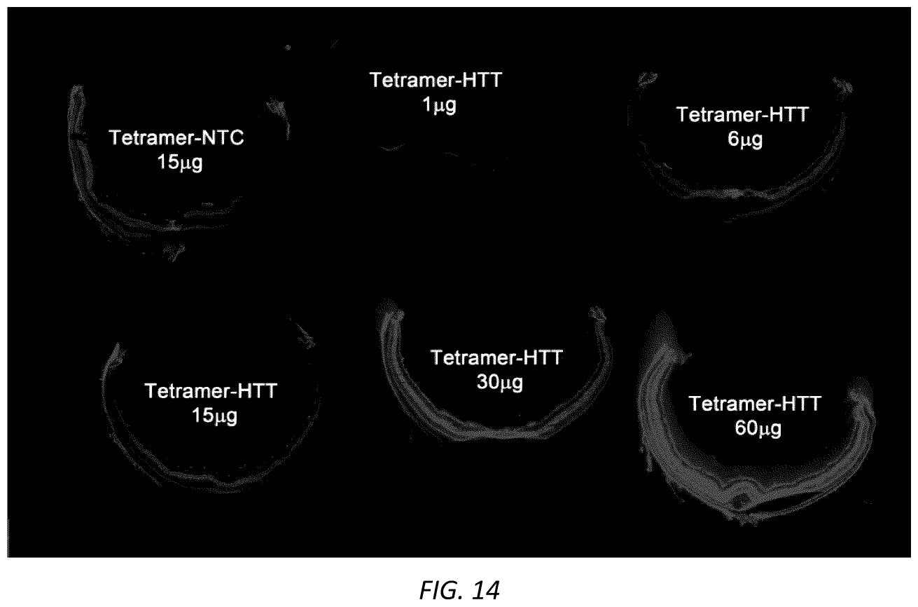

FIG. 14 displays retinal cross sections of eyes from the dose escalation study of the HTT knockdown in mouse with the Tetramer configuration whose results are shown in FIGS. 12 and 13. Images show Cy3 distribution across entire retinal section, indicating that the siRNA is taken up uniformly across the entire eye.

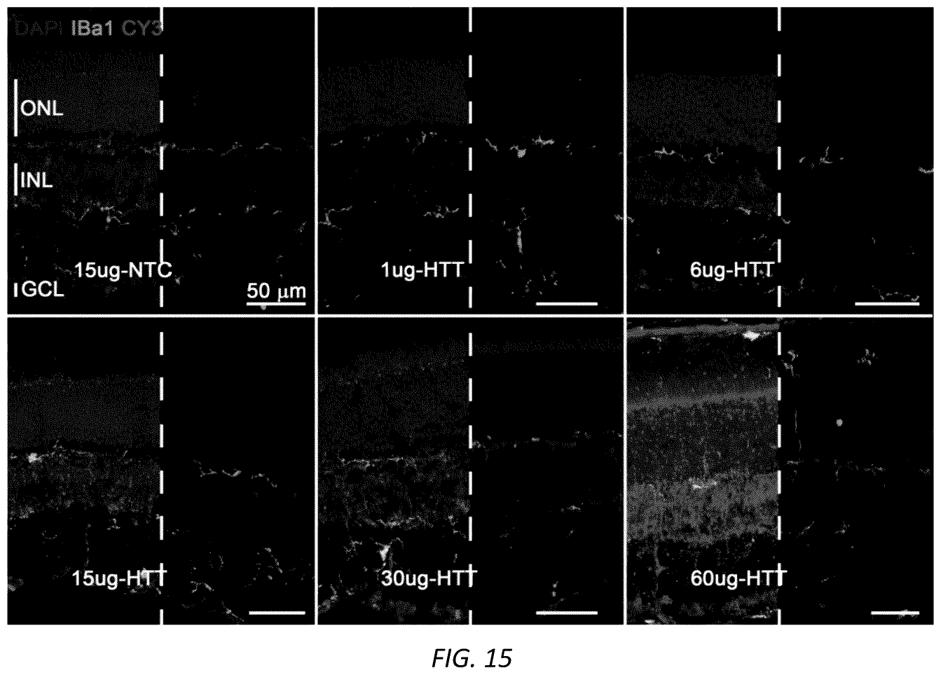

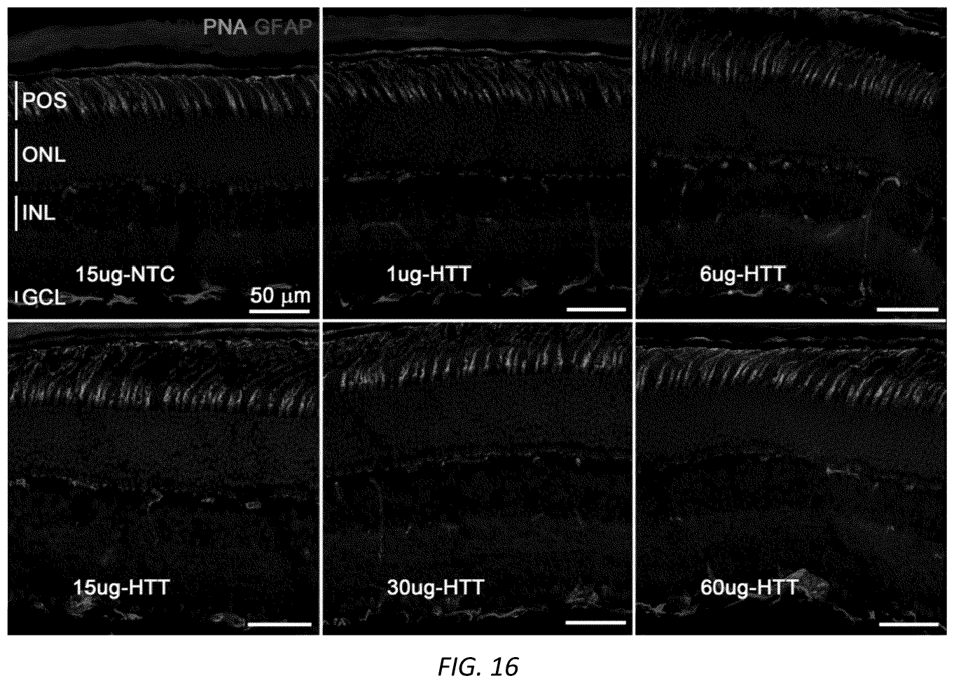

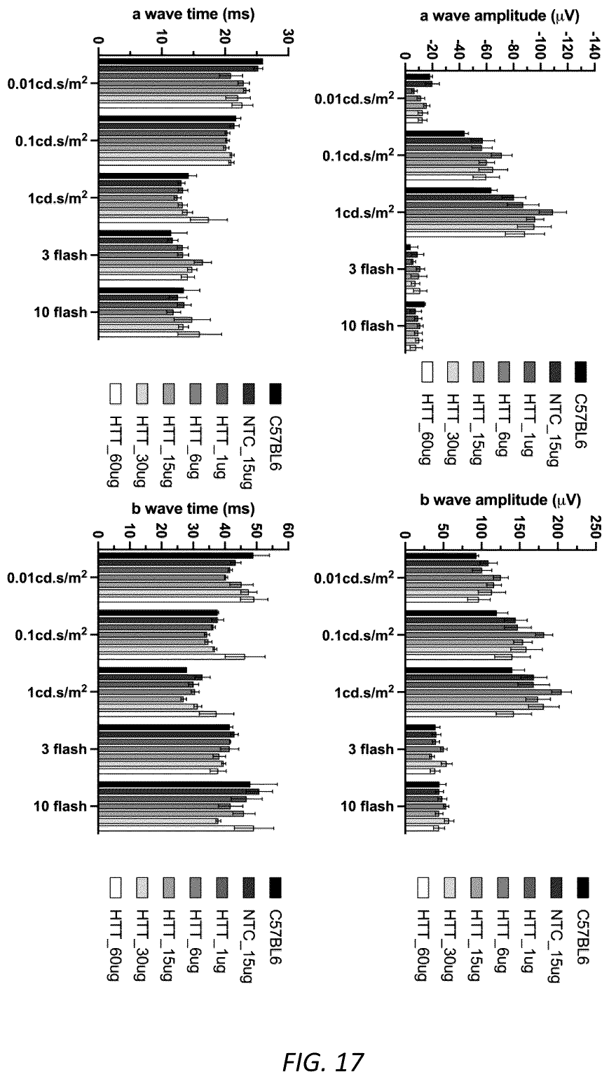

To determine toxicity, antibody staining was performed on retinal sections of eyes shown in FIG. 14 to identify Iba1 positive cells as well as changes in GFAP. FIG. 15 displays these retinal cross sections of eyes stained with Iba1 (green) to identify Iba1 positive cells that migrate to the outer nuclear layer (ONL) where photoreceptors reside. Iba1 positive cells in the ONL are seen at 60 microgram and occasionally at 30 microgram per injection indicating an inflammatory response at 60 microgram and a mild response at 30 microgram. Half of each panel (dotted line on FIG. 15) shows only the Iba1 signal to better visualize the signal Blue shows nuclear DAPI. FIG. 16 displays retinal cross sections of eyes from of dose escalation study shown in FIG. 14 stained with GFAP (red) to identify reactive gliosis in Müller glia cells. While a slight increase in GFAP expression is seen at the ganglion cell layer (GCL) level where astrocytes reside, the expression does not extent upwards into Müller glia cells. Expression of GFAP in astrocytes is normal. The expression is increased with 60 microgram, which is consistent with the results seen with Iba1. However, the absence of reactive gliosis indicates that there are no severe retinal degenerative events induced by the siRNA. siRNA is not shown as these are sections from the same eyes as shown in FIG. 15. In FIG. 16, the color blue indicates nuclear DAPI and green marks cone photoreceptor segment with peanut agglutinin lectin (PNA). FIG. 17 presents the measurements of photoreceptor and retinal function by electroretinography under scotopic (0.01cd.s/m2-1cd.s/m2) and photopic conditions (3 & 10 flashes). A-waves and b-waves recording show normal photoreceptor and inner retinal function, respectively for all amounts injected. There is no statistically significant difference between the recordings (n=5 mice per amount of siRNA) as seen in the upper two graphs. Implicit time of a- and b-waves (lower two graphs) are also not significantly different between the different groups of mice injected.

### Example 5: siRNA in Eye of a Large Animal Model: The Swine (All Data Shown Below is Generated With the Tetramer-Htt-siRNA-Cy3 and its NTC in Swine)

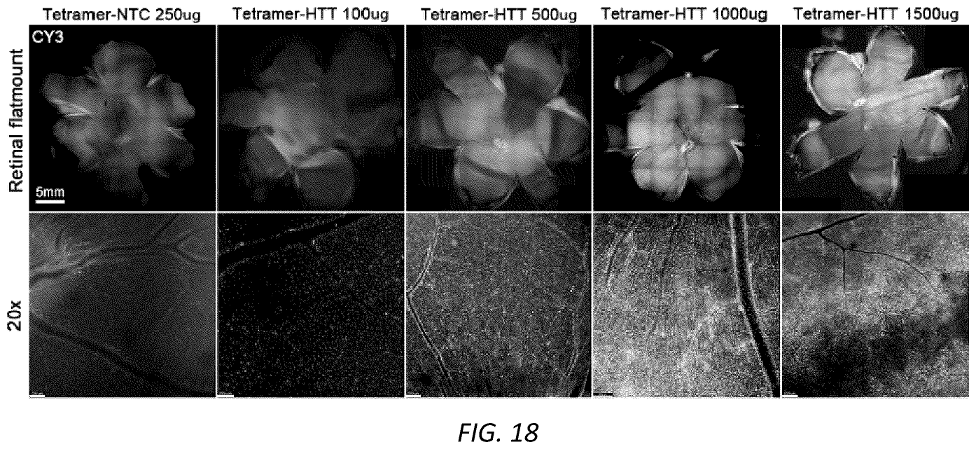

For translational purposes of the siRNA technology, the suitability of the technique in a large animal model was tested to determine distribution, knockdown efficiency and toxicity. To that end, the pig model was chosen as pigs have eyes similar in size to humans (35 kg pigs were used). The only difference is the absence of a fovea. As an initial test run, 3 pigs were injected with 5 different amounts of siRNA of the same chemical configuration, keeping the injection volume constant at 100 microliter. The following data represent a summary of the injections in pigs with the siRNA against HTT in the Tetramer configuration. All siRNA molecules were also labeled with Cy3. Pigs were euthanized 10 days after the intravitreal injection. FIG. 18 shows the fluorescence intensity of Tetramer-Htt-Cy3 after intravitreal delivery in pig eye. Delivery of amount of siRNA is shown on top of each panel in FIG. 18 (100-1500 microgram of Tetramer). Fluorescence intensity is well distributed across the entire eye. Top row in FIG. 18 shows the Cy3 fluorescence of unfixed tissue right after opening the eye, meanwhile the bottom panel is a higher magnification of a region from the top panel.

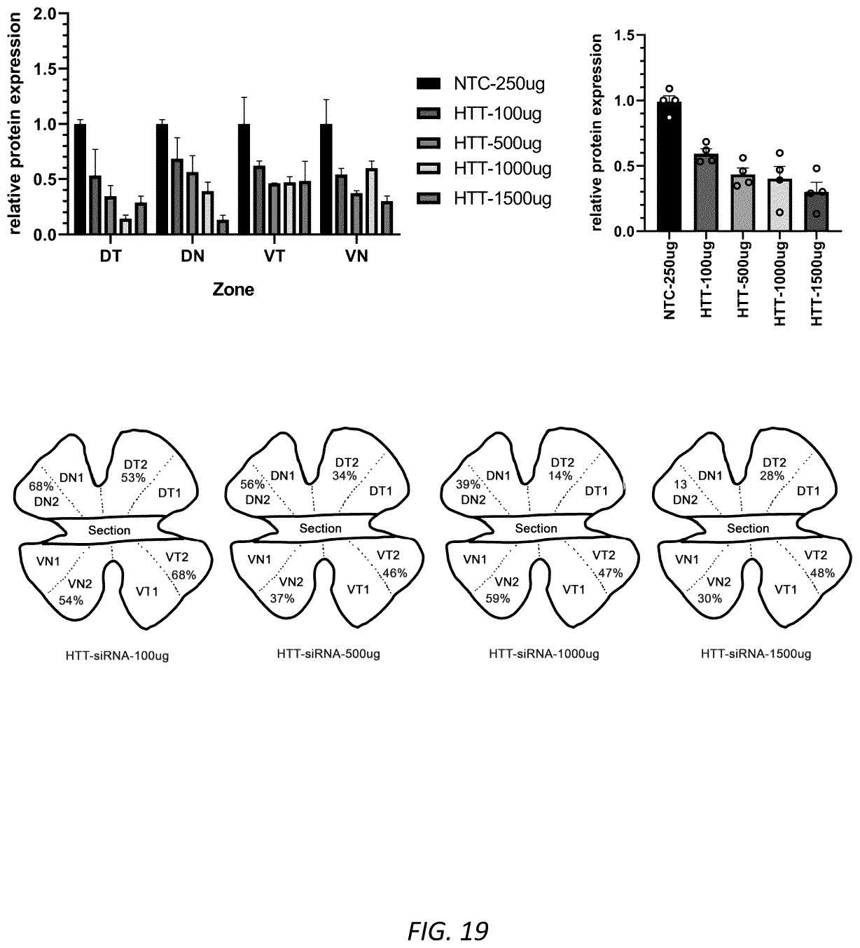

Knockdown of Huntington protein in Swine was measured from pigs' eyes shown in FIG. 18 by western blot analysis. Knockdown was compared to Huntington protein levels in the NTC that was injected with 250 ug of the Tetramer-siRNA-Cy3. In FIG. 19, Top figure shows knockdown in bar graphs seen in the four major retinal quadrants (DT: Dorsal-Temporal; DN: Dorsal-Nasal; VT: Temporal-Nasal; VN: Ventral-Nasal) with the error bars being generated by technical replicates. The knockdown efficiency in each quadrant is dependent on the positioning of the needle and the angle of insertion. Needles were generally inserted from the temporal side and pointed towards the center of the eye. Middle panel in FIG. 19 shows knockdown on a flat mount cartoon with corresponding values of the regional knockdown shown in the bar graph. Bottom panel in FIG. 19 shows the average knockdown of Huntington protein across the entire retina calculated by averaging the knockdown seen in each quadrant per retina with the error bars being generated by averaging the 4 data points for each quadrant per retina. Data shown in FIG. 19's bottom panel represents one biological sample for each amount of siRNA delivered.

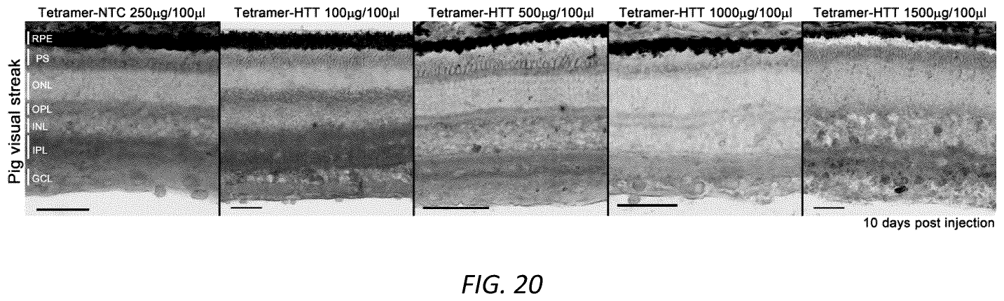

FIG. 20 displays antibody staining for Huntington protein on section of eyes injected with different amounts as shown in FIG. 19. The area of the sections of eyes is shown in the middle panel of FIG. 19. Huntington knockdown is seen across all retinal layers and in particular in the Inner and Outer Plexiform Layers (IPL, OPL), and where the photoreceptor segment (PS) is located.

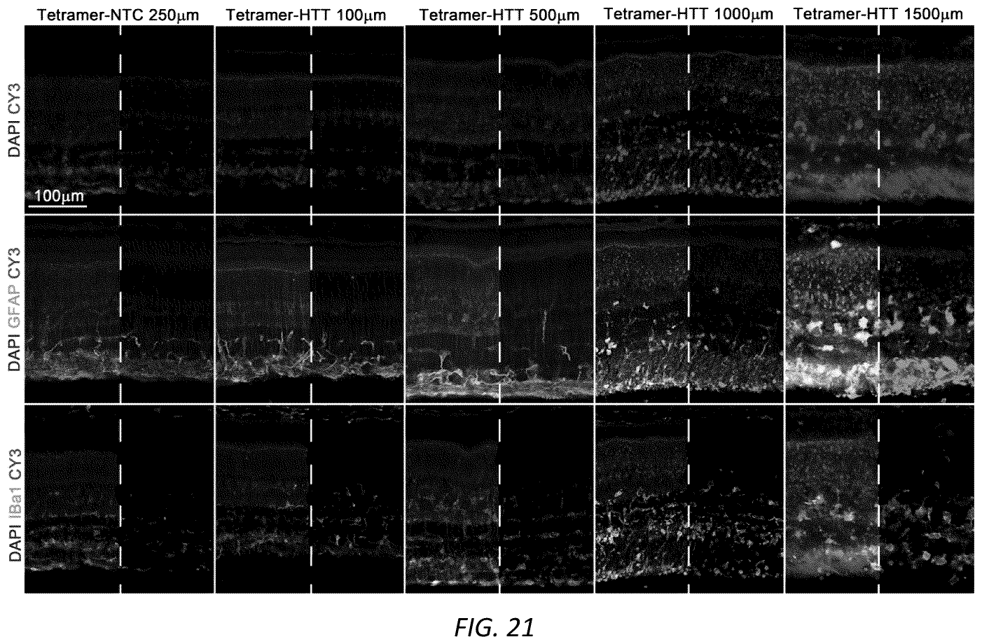

FIG. 21 displays antibody staining for GFAP (glial fibrillary acidic protein) and Iba1 (ionized calcium binding adaptor protein 1) (as shown in mouse on FIGS. 15 and 16) expression on retinal section of eyes injected with different amount as shown in FIGS. 18 and 19 to determine dose dependent toxicity. GFAP and Iba1 are both shown in green in FIG. 21 as indicated to the left of each row. Red staining in FIG. 21 shows siRNA distribution across the retinal section while nuclei are marked with nuclear DAPI. There is a clear dose dependent increase of GFAP and Iba1 expression. Up to 500 ug of siRNA expression of IBA1 and GFAP is rarely seen to progress into the outer nuclear layer (ONL) where photoreceptors reside. At 1000 ug and 1500 ug there is a visible increase in GFAP and Iba1 expression in the ONL. Additionally, a lot of the siRNA appears be taken up by Iba1 positive cells, which reflect likely macrophages that take up excess extracellular material. Half of each panel in FIG. 21 shows only the signal of interest (siRNA, GFAP or Iba1) to better visualize the signal.

Summary of Pig data: The Tetramer-Htt-siRNA distributes well across the entire retina after one intravitreal delivery in a large eye as the swine. This is particularly important as pig eyes are of similar size as human eyes. Besides lacking a fovea, the pig eye is the closest animal model to the human eye. For distribution studies it is more relevant due to the similar size when compared to most lab NHPs. The dose response in FIG. 19 and the toxicity in FIG. 21 show that for this particular compound doses in the range of 100-500 μg might be used for further studies. This should result to an approximate 50% knockdown of HTT which is similar to what is seen in mouse with the tetramer. Toxicity may be reduced by the removal of the Cy3 molecule, which was still attached in the current study.

### Example 6: Development of an siRNA Against S6K1 (RPS6KB1: Ribosomal Protein S6 Kinase B1

An initial bioinformatics screen was performed to identify potential siRNA sequences for S6K1. A list of sequences that were identified in the initial bioinformatics screen are shown below in Table 1 and Table 2.

TABLE 1
S6K1 45-nucleotide gene region target sites

| Oligo ID | Gene region |
|---|---|
| Rps6kb1_4456 | TTATTTTCTTTAAAATCAGCTATTACAGGATATTTT |
| | TTTATTTTC (SEQ ID NO: 13) |
| Rps6kb1_459 | AATACTGGGAAGATATTTGCCATGAAGGTGCTTAAA |
| | AAGGCAATG (SEQ ID NO: 1) |
| Rps6kb1_291 | GTTGGACCATATGAACTTGGCATGGAACATTGTGAG |
| | AAATTTGAA |
| | (SEQ ID NO: 2) |
| Rps6kb1_4893 | TTCCTAGGTTACAAGGGCTAGATCTAAGATTATTCT |
| | CATGAGAAA (SEQ ID NO: 14) |
| Rps6kb1_431 | TTTTCAAGTACGAAAAGTAACAGGAGCAAATACTGG |
| | GAAGATATT (SEQ ID NO: 15) |
| Rps6kb1_4561 | TAAGGTTTGTAGTGTTACAGAATAACTAAACTGGGA |
| | TTTATAAAC (SEQ ID NO: 16) |
| Rps6kb1_2104 | TTGCCTGTAATACTTGCAACTAAGGACAAATTAGCA |
| | TGCAAGCTT |
| | (SEQ ID NO: 3) |
| Rps6kb1_1217 | TCCCTTTAAGCCTCTGTTGCAATCTGAAGAGGATGT |
| | GAGTCAGTT (SEQ ID NO: 17) |
| Rps6kb1_2200 | ATTGATGTTTTACGTGCAAACAACCTGAATCTTTTT |
| | TTTATATAA |
| | (SEQ ID NO: 4) |
| Rps6kb1_1364 | ATCTGTACTTGAAAGTGTGAAAGAAAAGTTTTCATT |
| | TGAACCAAA |
| | (SEQ ID NO: 5) |
| Rps6kb1_3733 | GACTGTAGCAGCATTTTGAGAACTTCATAATTGTAG |
| | CAGTAAATT (SEQ ID NO: 18) |
| Rps6kb1_4801 | ATAAACAAAATGTATCTTAGCATTAATATCTTGAGC |
| | CTTGAACAT (SEQ ID NO: 19) |
| Rps6kb1_3133 | GGGTTTAATATCTGCAGAGCTTTATAAAATATACTG |
| | CAGTGCATA (SEQ ID NO: 20) |
| Rps6kb1_4476 | TATTACAGGATATTTTTTTATTTTCTACATTCTGTT |
| | TTTTAATTA (SEQ ID NO: 21) |
| Rps6kb1_4378 | TTCTAAGAGAGGTGTTCATGCTTGTACCAGGTAAGT |
| | GAATAAAAA (SEQ ID NO: 22) |
| Rps6kb1_2809 | TACTGTTATGCTGTTTACCTTCCTTAACAATTTTC |
| | TTTTTTGAGA (SEQ ID NO: 23) |
| Rps6kb1_2539 | CGTAACTGCAAGCCTTGGGACAGGCAGAAGTTGTAT |
| | GATCTACAT (SEQ ID NO: 24) |
| Rps6kb1_4323 | TTTATTTGTAAAAACTGAAGCATAATTTAAAGTGTA |
| | TATCAATAC (SEQ ID NO: 25) |
| Rps6kb1_2049 | CCGTTGAAATCTGATGATGTCAAATAAGGGTTATCC |
| | TAATAGGCA |
| | (SEQ ID NO: 6) |
| Rps6kb1_3965 | AATTCCAATAAAACTTTTACACTAAATGATTCCTCC |
| | TTAGCCCTA (SEQ ID NO: 26) |
| Rps6kb1_1893 | AAACCCACAAAAAACTCAAGCAAAATAGTATTGTGG |
| | AATCCACAG (SEQ ID NO: 27) |

For the above recited 45-nucleotide gene regions, the sequences correspond to the DNA gene sequence, however the mRNA encoded by the S6K1 gene will have the same sequences with T nucleotides replaced with U nucleotides. Accordingly, and by way of example, an siRNA with an antisense strand that targets SEQ ID NO: 1 will target the mRNA sequence that corresponds to the gene region of SEQ ID NO: 1.

TABLE 2
S6K1 20-nucleotide target sites

| Oligo ID | Sequence |
|---|---|
| Rps6kb1_4456 | UCAGCUAUUACAGGAUAUUU (SEQ ID NO: 28) |
| Rps6kb1_459 | UUUGCCAUGAAGGUGCUUAA (SEQ ID NO: 7) |
| Rps6kb1_291 | CUUGGCAUGGAACAUUGUGA (SEQ ID NO: 8) |
| Rps6kb1_4893 | GGCUAGAUCUAAGAUUAUUC (SEQ ID NO: 29) |
| Rps6kb1_431 | AGUAACAGGAGCAAAUACUG (SEQ ID NO: 30) |
| Rps6kb1_4561 | UACAGAAUAACUAAACUGGG (SEQ ID NO: 31) |
| Rps6kb1_2104 | GCAACUAAGGACAAAUUAGC (SEQ ID NO: 9) |
| Rps6kb1_1217 | GUUGCAAUCUGAAGAGGAUG (SEQ ID NO: 32) |
| Rps6kb1_2200 | GCAAACAACCUGAAUCUUUU (SEQ ID NO: 10) |
| Rps6kb1_1364 | UGUGAAAGAAAAGUUUUCAU (SEQ ID NO: 11) |
| Rps6kb1_3733 | UUGAGAACUUCAUAAUUGUA (SEQ ID NO: 33) |
| Rps6kb1_4801 | CUUAGCAUUAAUAUCUUGAG (SEQ ID NO: 34) |
| Rps6kb1_3133 | AGAGCUUUAUAAAAUAUACU (SEQ ID NO: 35) |
| Rps6kb1_4476 | UUUUAUUUUCUACAUUCUGU (SEQ ID NO: 36) |
| Rps6kb1_4378 | UCAUGCUUGUACCAGGUAAG (SEQ ID NO: 37) |
| Rps6kb1_2809 | UACCUUCCUUAACAAUUUUC (SEQ ID NO: 38) |
| Rps6kb1_2539 | UGGGACAGGCAGAAGUUGUA (SEQ ID NO: 39) |
| Rps6kb1_4323 | UGAAGCAUAAUUUAAAGUGU (SEQ ID NO: 40) |
| Rps6kb1_2049 | GAUGUCAAAUAAGGGUUAUC (SEQ ID NO: 12) |
| Rps6kb1_3965 | UUUACACUAAAUGAUUCCUC (SEQ ID NO: 41) |
| Rps6kb1_1893 | UCAAGCAAAAUAGUAUUGUG (SEQ ID NO: 42) |

TABLE 3
S6K1 sense strands

| Duplex | Oligo | |
|---|---|---|
| # | ID | Sense Sequence |
| 1 | Rps6kb1_ | (mU)#(mA)#(mU)(mU)(fA)(fC)(fA)(mG)(fG)(mA)(mU)(mA) |
| | 4456 | (mU)#(mU)#(mA)-TegChol (SEQ ID NO: 43) |
| 2 | Rps6kb1_ | (mC)#(mA)#(mU)(mG)(fA)(fA)(fG)(mG)(fU)(mG)(mC)(mU) |
| | 459 | (mU)#(mA)#(mA)-TegChol (SEQ ID NO: 44) |
| 3 | Rps6kb1_ | (mC)#(mA)#(mU)(mG)(fG)(fA)(fA)(mC)(fA)(mU)(mU)(mG) |
| | 291 | (mU)#(mG)#(mA)-TegChol (SEQ ID NO: 45) |
| 4 | Rps6kb1_ | (mG)#(mA)#(mU)(mC)(fU)(fA)(fA)(mG)(fA)(mU)(mU)(mA) |
| | 4893 | (mU)#(mU)#(mA)-TegChol (SEQ ID NO: 46) |
| 5 | Rps6kb1_ | (mC)#(mA)#(mG)(mG)(fA)(fG)(fC)(mA)(fA)(mA)(mU)(mA) |
| | 431 | (mC)#(mU)#(mA)-TegChol (SEQ ID NO: 47) |
| 6 | Rps6kb1_ | (mA)#(mA)#(mU)(mA)(fA)(fC)(fU)(mA)(fA)(mA)(mC)(mU) |
| | 4561 | (mG)#(mG)#(mA)-TegChol (SEQ ID NO: 48) |
| 7 | Rps6kb1_ | (mU)#(mA)#(mA)(mG)(fG)(fA)(fC)(mA)(fA)(mA)(mU)(mU) |
| | 2104 | (mA)#(mG)#(mA)-TegChol (SEQ ID NO: 49) |
| 8 | Rps6kb1_ | (mA)#(mA)#(mU)(mC)(fU)(fG)(fA)(mA)(fG)(mA)(mG)(mG) |
| | 1217 | (mA)#(mU)#(mA)-TegChol (SEQ ID NO: 50) |
| 9 | Rps6kb1_ | (mC)#(mA)#(mA)(mC)(fC)(fU)(fG)(mA)(fA)(mU)(mC)(mU) |
| | 2200 | (mU)#(mU)#(mA)-TegChol (SEQ ID NO: 51) |
| 10 | Rps6kb1_ | (mA)#(mA)#(mG)(mA)(fA)(fA)(fA)(mG)(fU)(mU)(mU)(mU) |
| | 1364 | (mC)#(mA)#(mA)-TegChol (SEQ ID NO: 52) |
| 11 | Rps6kb1_ | (mA)#(mA)#(mC)(mU)(fU)(fC)(fA)(mU)(fA)(mA)(mU)(mU) |
| | 3733 | (mG)#(mU)#(mA)-TegChol (SEQ ID NO: 53) |
| 12 | Rps6kb1_ | (mC)#(mA)#(mU)(mU)(fA)(fA)(fU)(mA)(fU)(mC)(mU)(mU) |
| | 4801 | (mG)#(mA)#(mA)-TegChol (SEQ ID NO: 54) |
| 13 | Rps6kb1_ | (mU)#(mU)#(mU)(mA)(fU)(fA)(fA)(mA)(fA)(mU)(mA)(mU) |
| | 3133 | (mA)#(mC)#(mA)-TegChol (SEQ ID NO: 55) |
| 14 | Rps6kb1_ | (mU)#(mU)#(mU)(mU)(fC)(fU)(fA)(mC)(fA)(mU)(mU)(mC) |
| | 4476 | (mU)#(mG)#(mA)-TegChol (SEQ ID NO: 56) |
| 15 | Rps6kb1_ | (mC)#(mU)#(mU)(mG)(fU)(fA)(fC)(mC)(fA)(mG)(mG)(mU) |
| | 4378 | (mA)#(mA)#(mA)-TegChol(SEQ ID NO: 57) |
| 16 | Rps6kb1_ | (mU)#(mC)#(mC)(mU)(fU)(fA)(fA)(mC)(fA)(mA)(mU)(mU) |
| | 2809 | (mU)#(mU)#(mA)-TegChol (SEQ ID NO: 58) |
| 17 | Rps6kb1_ | (mC)#(mA)#(mG)(mG)(fC)(fA)(fG)(mA)(fA)(mG)(mU)(mU) |
| | 2539 | (mG)#(mU)#(mA)-TegChol (SEQ ID NO: 59) |
| 18 | Rps6kb1_ | (mC)#(mA)#(mU)(mA)(fA)(fU)(fU)(mU)(fA)(mA)(mA)(mG) |
| | 4323 | (mU)#(mG)#(mA)-TegChol (SEQ ID NO: 60) |
| 19 | Rps6kb1_ | (mC)#(mA)#(mA)(mA)(fU)(fA)(fA)(mG)(fG)(mG)(mU)(mU) |
| | 2049 | (mA)#(mU)#(mA)-TegChol (SEQ ID NO: 61) |
| 20 | Rps6kb1_ | (mA)#(mC)#(mU)(mA)(fA)(fA)(fU)(mG)(fA)(mU)(mU)(mC) |
| | 3965 | (mC)#(mU)#(mA)-TegChol (SEQ ID NO: 62) |
| 21 | Rps6kb1_ | (mC)#(mA)#(mA)(mA)(fA)(fU)(fA)(mG)(fU)(mA)(mU)(mU) |
| | 1893 | (mG)#(mU)#(mA)-TegChol (SEQ ID NO: 63) |

TABLE 4
S6K1 sense strands

| Duplex | Oligo | |
|---|---|---|
| # | ID | Antisense Sequence |
| 1 | Rps6kb1_ | P(mU)#(fA)#(mA)(mU)(mA)(fU)(mC)(mC)(mU)(mG)(mU)(mA) |
| | 4456 | (mA)#(fU)#(mA)#(fG)#(mC)#(mU)#(mG)#(fA) (SEQ ID NO: 64) |
| 2 | Rps6kb1_ | P(mU)#(fU)#(mA)(mA)(mG)(fC)(mA)(mC)(mC)(mU)(mU)(mC) |
| | 459 | (mA)#(fU)#(mG)#(fG)#(mC)#(mA)#(mA)#(fA) (SEQ ID NO: 65) |
| 3 | Rps6kb1_ | P(mU)#(fC)#(mA)(mC)(mA)(fA)(mU)(mG)(mU)(mU)(mC)(mC) |
| | 291 | (mA)#(fU)#(mG)#(fC)#(mC)#(mA)#(mA)#(fG) (SEQ ID NO: 66) |
| 4 | Rps6kb1_ | P(mU)#(fA)#(mA)(mU)(mA)(fA)(mU)(mC)(mU)(mU)(mA)(mG) |
| | 4893 | (mA)#(fU)#(mC)#(fU)#(mA)#(mG)#(mC)#(fC) (SEQ ID NO: 67) |
| 5 | Rps6kb1_ | P(mU)#(fA)#(mG)(mU)(mA)(fU)(mU)(mU)(mG)(mC)(mU)(mC) |
| | 431 | (mC)#(fU)#(mG)#(fU)#(mU)#(mA)#(mC)#(fU) (SEQ ID NO: 68) |
| 6 | Rps6kb1_ | P(mU)#(fC)#(mC)(mA)(mG)(fU)(mU)(mU)(mA)(mG)(mU)(mU) |
| | 4561 | (mA)#(fU)#(mU)#(fC)#(mU)#(mG)#(mU)#(fA) (SEQ ID NO: 69) |
| 7 | Rps6kb1_ | P(mU)#(fC)#(mU)(mA)(mA)(fU)(mU)(mU)(mG)(mU)(mC)(mC) |
| | 2104 | (mU)#(fU)#(mA)#(fG)#(mU)#(mU)#(mG)#(fC) (SEQ ID NO: 70) |
| 8 | Rps6kb1_ | P(mU)#(fA)#(mU)(mC)(mC)(fU)(mC)(mU)(mU)(mC)(mA)(mG) |
| | 1217 | (mA)#(fU)#(mU)#(fG)#(mC)#(mA)#(mA)#(fC) (SEQ ID NO: 71) |
| 9 | Rps6kb1_ | P(mU)#(fA)#(mA)(mA)(mG)(fA)(mU)(mU)(mC)(mA)(mG)(mG) |
| | 2200 | (mU)#(fU)#(mG)#(fU)#(mU)#(mU)#(mG)#(fC) (SEQ ID NO: 72) |
| 10 | Rps6kb1_ | P(mU)#(fU)#(mG)(mA)(mA)(fA)(mA)(mC)(mU)(mU)(mU)(mU) |
| | 1364 | (mC)#(fU)#(mU)#(fU)#(mC)#(mA)#(mC)#(fA) (SEQ ID NO: 73) |
| 11 | Rps6kb1_ | P(mU)#(fA)#(mC)(mA)(mA)(fU)(mU)(mA)(mU)(mG)(mA)(mA) |
| | 3733 | (mG)#(fU)#(mU)#(fC)#(mU)#(mC)#(mA)#(fA) (SEQ ID NO: 74) |
| 12 | Rps6kb1_ | P(mU)#(fU)#(mC)(mA)(mA)(fG)(mA)(mU)(mA)(mU)(mU)(mA) |
| | 4801 | (mA)#(fU)#(mG)#(fC)#(mU)#(mA)#(mA)#(fG) (SEQ ID NO: 75) |
| 13 | Rps6kb1_ | P(mU)#(fG)#(mU)(mA)(mU)(fA)(mU)(mU)(mU)(mU)(mA)(mU) |
| | 3133 | (mA)#(fA)#(mA)#(fG)#(mC)#(mU)#(mC)#(fU) (SEQ ID NO: 76) |
| 14 | Rps6kb1_ | P(mU)#(fC)#(mA)(mG)(mA)(fA)(mU)(mG)(mU)(mA)(mG)(mA) |
| | 4476 | (mA)#(fA)#(mA)#(fU)#(mA)#(mA)#(mA)#(fA) (SEQ ID NO: 77) |
| 15 | Rps6kb1_ | P(mU)#(fU)#(mU)(mA)(mC)(fC)(mU)(mG)(mG)(mU)(mA)(mC) |
| | 4378 | (mA)#(fA)#(mG)#(fC)#(mA)#(mU)#(mG)#(fA) (SEQ ID NO: 78) |
| 16 | Rps6kb1_ | P(mU)#(fA)#(mA)(mA)(mA)(fU)(mU)(mG)(mU)(mU)(mA)(mA) |
| | 2809 | (mG)#(fG)#(mA)#(fA)#(mG)#(mG)#(mU)#(fA) (SEQ ID NO: 79) |
| 17 | Rps6kb1_ | P(mU)#(fA)#(mC)(mA)(mA)(fC)(mU)(mU)(mC)(mU)(mG)(mC) |
| | 2539 | (mC)#(fU)#(mG)#(fU)#(mC)#(mC)#(mC)#(fA) (SEQ ID NO: 80) |
| 18 | Rps6kb1_ | P(mU)#(fC)#(mA)(mC)(mU)(fU)(mU)(mA)(mA)(mA)(mU)(mU) |
| | 4323 | (mA)#(fU)#(mG)#(fC)#(mU)#(mU)#(mC)#(fA) (SEQ ID NO: 81) |
| 19 | Rps6kb1_ | P(mU)#(fA)#(mU)(mA)(mA)(fC)(mC)(mC)(mU)(mU)(mA)(mU) |
| | 2049 | (mU)#(fU)#(mG)#(fA)#(mC)#(mA)#(mU)#(fC) (SEQ ID NO: 82) |
| 20 | Rps6kb1_ | P(mU)#(fA)#(mG)(mG)(mA)(fA)(mU)(mC)(mA)(mU)(mU)(mU) |
| | 3965 | (mA)#(fG)#(mU)#(fG)#(mU)#(mA)#(mA)#(fA) (SEQ ID NO: 83) |
| 21 | Rps6kb1_ | P(mU)#(fA)#(mC)(mA)(mA)(fU)(mA)(mC)(mU)(mA)(mU)(mU) |
| | 1893 | (mU)#(fU)#(mG)#(fC)#(mU)#(mU)#(mG)#(fA) (SEQ ID NO: 84) |

For the sense and antisense sequences of Table 3 and 3, “m” corresponds to a 2′-O-methyl modified nucleotide, “f” corresponds to a 2′-fluoro modified nucleotide, “#” corresponds to a phosphorothioate internucleotide linkage, “P” corresponds to a 5′ phosphate, and “TegChol” corresponds to a tri- or tetra-ethylene glycol linked cholesterol moiety.

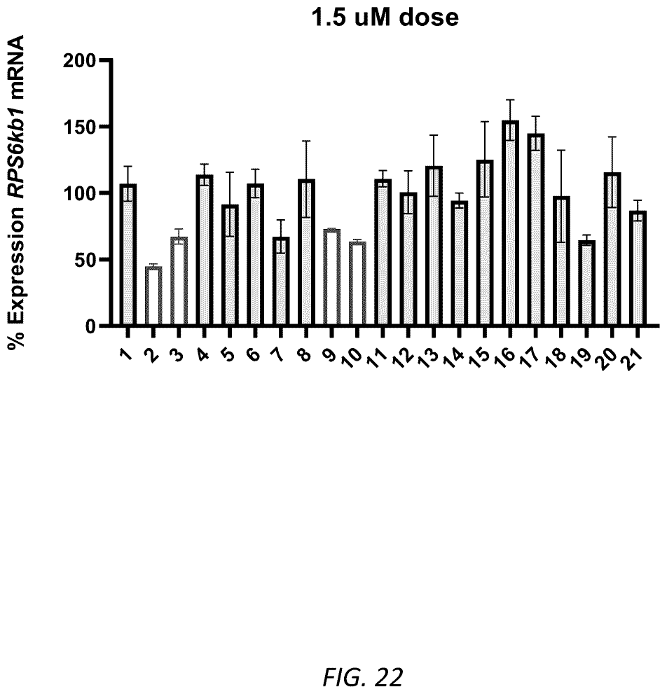

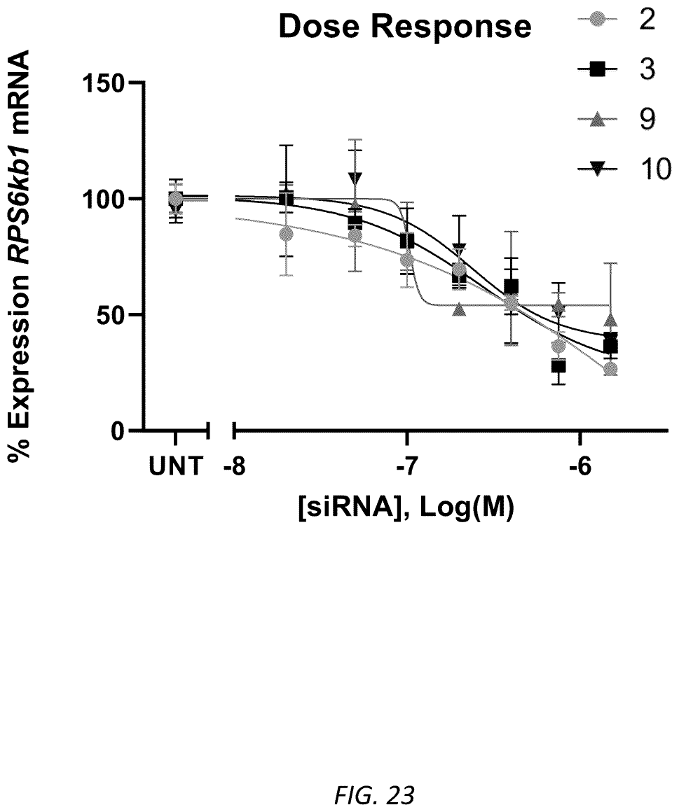

FIG. 22 shows the initial knockdown efficiency in vitro of the duplexes formed from the sense and antisense strands shown in Table 3 and 4. Candidates with the best knockdown results were duplexes 2, 3, 7, 9, 10, and 19. FIG. 23 shows dose response curves for the 4 sequences highlighted in red in FIG. 22. Duplex 2 (Rps6k1b_459) showed the most consistent response and was therefore selected for further studies in vivo.

The primary objective of the siRNA for S6K1 is to knockdown S6K1 in photoreceptors for the treatment of AMD. Based on the data generated by the different HTT-siRNA conjugates, an siRNA was developed initially in the tetramer configuration without any Cy3 label to reduce toxicity. In vivo data from mouse and NHP was then generated, based on the tetramer configuration for duplex 2 (Rps6k1b_459).

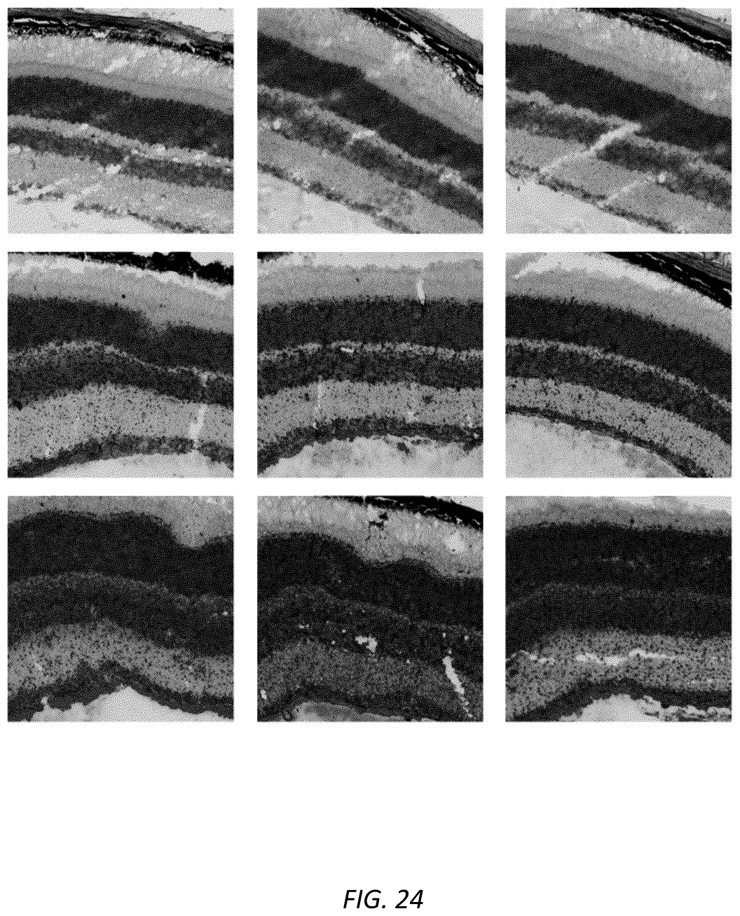

FIG. 24 shows an RNA-Scope in situ hybridization on retinal cross-sections of mice to detect the siRNA-Tetramer against S6K1. The top row in FIG. 24 shows sections from 3 mice injected with the NTC for S6K1 in the tetramer configuration; the middle row shows sections from 3 mice injected with the 3 μg/eye with the siRNA against S6K1 in the tetramer configuration; and the last row shows sections from 3 mice injected with the 6 ug/eye with the siRNA against S6K1 in the tetramer configuration. siRNA was delivered intravitreally and animals were euthanized 2 weeks post injection.

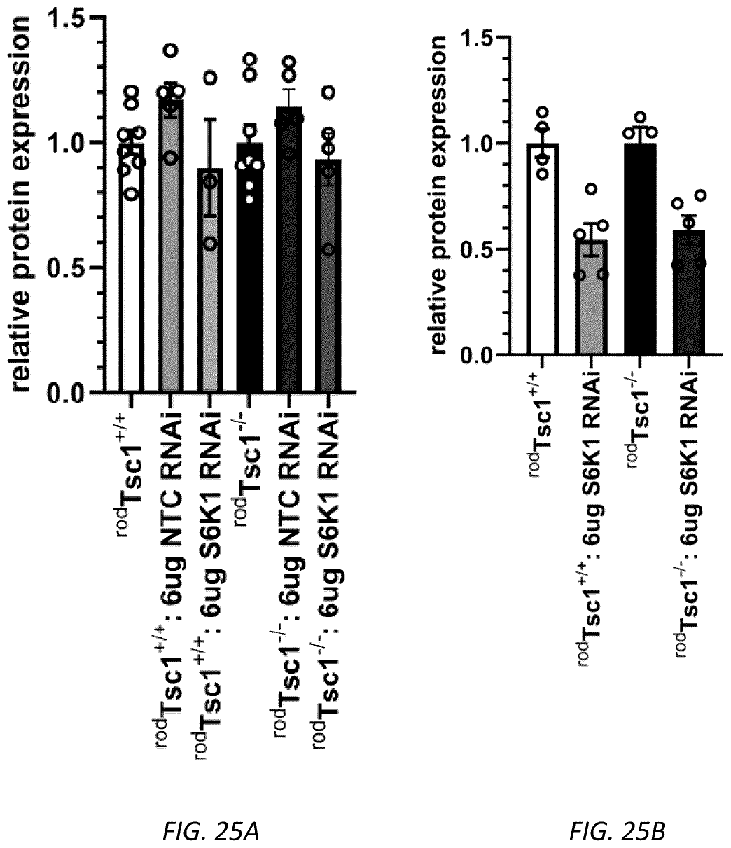

FIG. 25A and FIG. 25B display knockdown of S6K1 in mouse after intravitreal injection of 6 μg of siRNA in the Tetramer configuration. Both graphs in FIG. 25 use rodTSC1−/− mice that have been shown to develop age-related macular degeneration like pathologies. The rodTSC1+/+ mice serve are Cre-negative littermate controls that do not develop pathologies. FIG. 25A shows S6K1 protein levels as detected by western blot 2-weeks post injection. A small tendency of reduced S6K1 protein is seen when compared to uninjected littermates or NTC mice. FIG. 25B shows similar data as first graph at 2 months post-injection. A strong knockdown is seen (40-45%) with 6 μg of siRNA. Each dot in the graphs in FIG. 25 represent one biological sample (retina) from one animal.

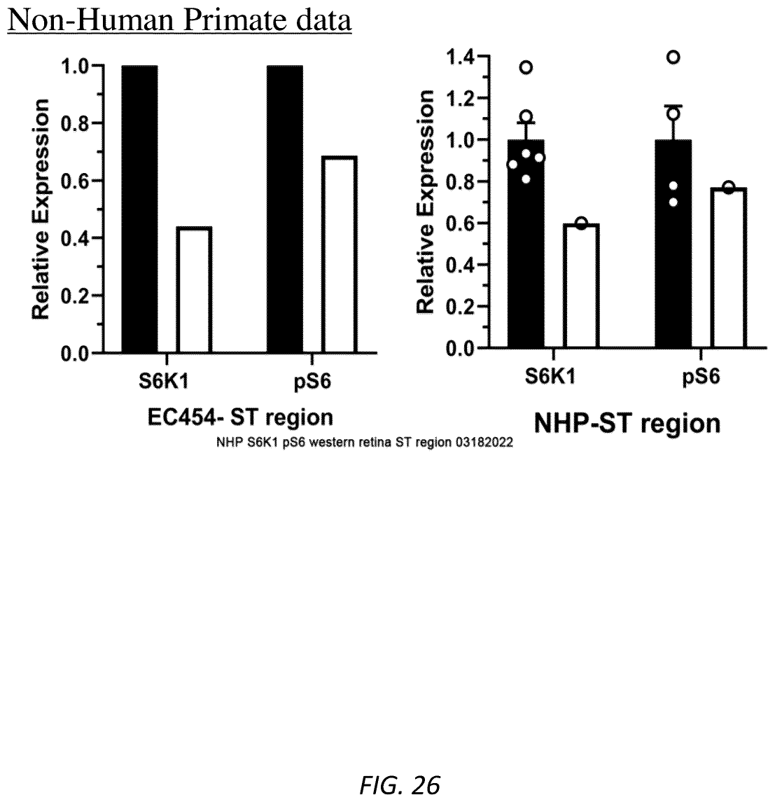

FIG. 26 shows the knockdown of S6K1 protein in non-human primate (NHP). Western blot data with retinal protein extracts form the superior-temporal (ST) regions (AKA: dorsal-temporal) of one NHP injected intravitreally with 225 μg of S6K1-tetramer (in 75 uL) and 6 naïve NHP retinas from the same region. First set of bar graphs shows a comparison between the uninjected contralateral eye and the S6K1 siRNA injected one to allow for a direct intra-animal comparison between both eyes. The second bar graph shows a comparison between the 6 naïve NHPs and the S6K1 siRNA injected one. The knockdown efficiency of S6K1 appears in both cases around 50%. NHP eyes were harvested 1-month post-injection. Shown is also the decrease in phosphorylation of ribosomal protein S6, which is a canonical target of S6K1. Similar to the S6K1 knockdown data, intra-animal comparison is shown to the left and comparison with several NHPs is shown to the right.

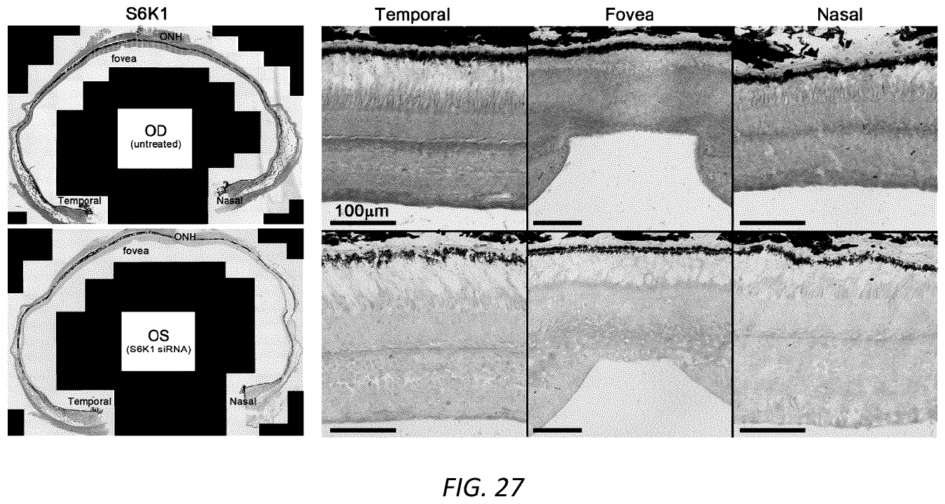

FIG. 27 displays the knockdown of S6K1 protein on retinal cross section of non-human primate (NHP) after siRNA treatment. Data is generated with the one injected eye (see also FIG. 26) and the uninjected contralateral eye. Sections were obtained from the central regions as shown for the pig in FIG. 19. Entire cross section encompassing the fovea are shown to the left in FIG. 27. Higher magnification of temporal and nasal regions as well as the fovea are shown to the right in FIG. 27. Top row of FIG. 27 shows uninjected eye and bottom row eye injected intravitreally with 225 μg of S6K1-tetramer (in 75 μL). Consistent with the western blot data presented in FIG. 26 that was generated with the superior-temporal region of the same eye, there is a clear reduction in signal for S6K1 protein expression. The reduction is also noticeable in the fovea, which contains only cones, indicating the knockdown in cones is equally efficient as in rods.

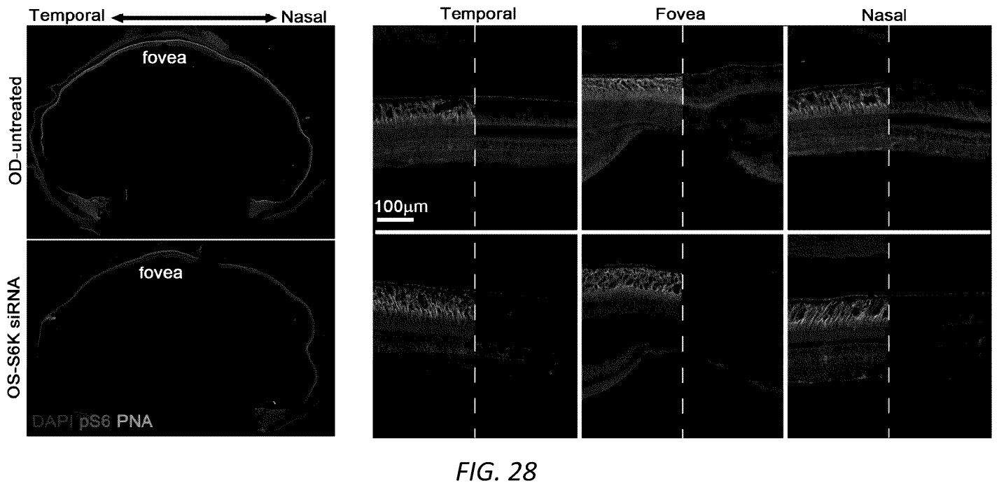

FIG. 28 shows the reduction in phosphorylated S6 protein (pS6) on retinal cross section of non-human primate (NHP) after siRNA treatment. Data in FIG. 28 is the same as shown in FIG. 27, with the exception that the staining probes for the expression of pS6 (red signal). A clear decrease of pS6 is seen across the entire retina, in particular also in photoreceptors, including foveal cones. In each panel in FIG. 28 green and blue signals have been removed from half the panel (dotted line) to better visualize the knockdown of pS6. Blue color in FIG. 28 shows nuclear DAPI while green shows cones segments marked with peanut agglutinin lectin (PNA).

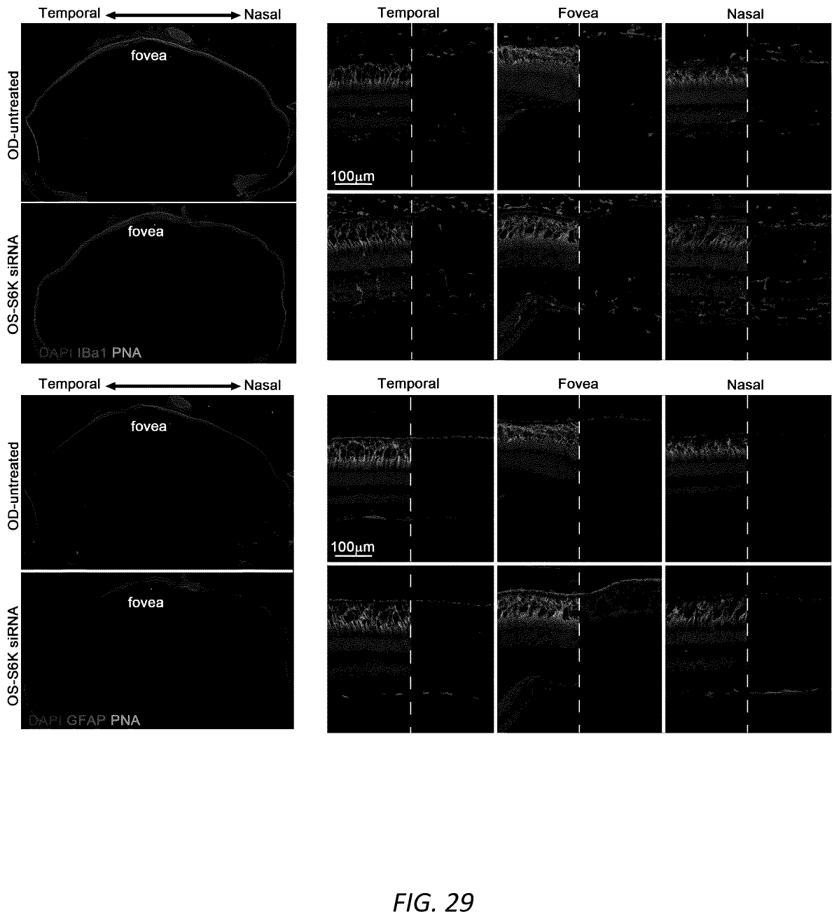

FIG. 29 shows the expression of inflammatory markers in NHP after siRNA treatment with S6K1 siRNA (75 μL, 225 μg of siRNA in tetramer configuration). Data in FIG. 29 is the same as shown in FIGS. 27 and 28, with the exception that the staining probes for the expression of Iba1 (red signal, first set) and GFAP (red signal, second set). Untreated contralateral eye is in first row of each set and the treated one in the second row. There is a slight increase in Iba1 positive cells that migrate towards the photoreceptor layer in the siRNA treated eye. However, there is no reactive gliosis as seen by GFAP staining. The staining is only slightly increased where astrocytes reside in the ganglion cell layer. There is no increase in the fovea. The data presented in FIG. 29 suggests that is no severe adverse reaction to the siRNA treatment of the knockdown of S6K1. In each panel in FIG. 29, green and blue signal have been removed from half the panel (dotted line) to better visualize the Iba1 and GFAP signal. In FIG. 29, the blue color indicates nuclear DAPI meanwhile the green color indicates cones segments marked with peanut agglutinin lectin (PNA).

Summary on S6K1-siRNA data. The Tetramer-S6K1-siRNA distributes well across the entire retina after one intravitreal delivery of 6 μg in the mouse eye. Knockdown efficiency appears slow initially but very robust over time. Therapeutically, ˜50% knockdown of S6K1 protein in photoreceptors needs to be attained, which seems to be achievable. Duplex 2 selected from the initial screen worked very efficiently in vivo, demonstrating that the S6K1 target site of SEQ ID NO: 1 is a useful target for the knockdown of S6K1. Based on the dose response curve for HTT-Tetramer in mouse, an injection of 25 μg/eye in a subset of mice will be performed and analyzed in the near future for S6K1 knockdown and for markers of age-related macular degeneration, to determine if disease progression is ameliorated. Injections in NHP confirm that knockdown is working equally efficient in a large eye, that distribution is widespread, the therapeutic range can be achieved and that there is no serious inflammatory response to the treatment. Overall, the data shows that gene knockdown in humans is feasible for the treatment of various retinal diseases.

1. An siRNA comprising a sense strand and an antisense strand, wherein the antisense strand comprises a sequence substantially complementary to a S6K1 nucleic acid sequence of any one of SEQ ID NOs: 1-6.

2. The siRNA of claim 1, wherein the antisense strand comprises a sequence substantially complementary to a S6K1 nucleic acid sequence of any one of SEQ ID NOs: 7-12.

3. The siRNA of claim 1, comprising complementarity to at least 10, 11, 12 or 13 contiguous nucleotides of the S6K1 nucleic acid sequence of any one of SEQ ID NOs: 1-6.

4. The siRNA of claim 1, comprising no more than 3 mismatches with the S6K1 nucleic acid sequence of any one of SEQ ID Nos: 1-6.

5. (canceled)

6. The siRNA of claim 1, wherein:
the antisense strand and/or the sense strand comprise about 15 nucleotides to 25 nucleotides in length;
the siRNA comprises a double-stranded region of 15 base pairs to 20 base pairs;
the siRNA comprises at least one blunt-end;
the siRNA comprises least one single stranded nucleotide overhang; and/or
the siRNA comprises naturally occurring nucleotides.

7-25. (canceled)

26. The siRNA of claim 1, wherein:
the siRNA comprises at least one modified nucleotide;
the siRNA comprises at least one modified internucleotide linkage;
the sense strand comprises one or more nucleotide mismatches between the antisense strand and the sense strand; and/or
a functional moiety is linked to a 5′ end and/or 3′ end of the antisense strand and/or sense strand.

27. The siRNA of claim 26, wherein the siRNA comprises at least one modified nucleotide, wherein said modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof

28. (canceled)

29. The siRNA of claim 26, wherein the siRNA comprises at least one modified internucleotide linkage, wherein said modified internucleotide linkage comprises a phosphorothioate internucleotide linkage.

30-47. (canceled)

48. The siRNA of claim 26, wherein the functional moiety is linked to the 5′ end and/or 3′ end of the antisense strand and/or sense strand, and wherein:
the functional moiety comprises a hydrophobic moiety; or
the functional moiety comprises a hydrophobic moiety selected from the group consisting of fatty acids, steroids, secosteroids, lipids, gangliosides, nucleoside analogs, endocannabinoids, vitamins, and a mixture thereof.

49-54. (canceled)

55. The siRNA of claim 26, wherein the functional moiety is linked to the 5′ end and/or 3′ end of the antisense strand and/or sense strand, and wherein the functional moiety is linked to the antisense strand and/or sense strand by a linker.

56. The siRNA of claim 55, wherein the linker comprises a divalent or trivalent linker.

57. The siRNA of claim 56, wherein the divalent or trivalent linker is selected from the group consisting of: 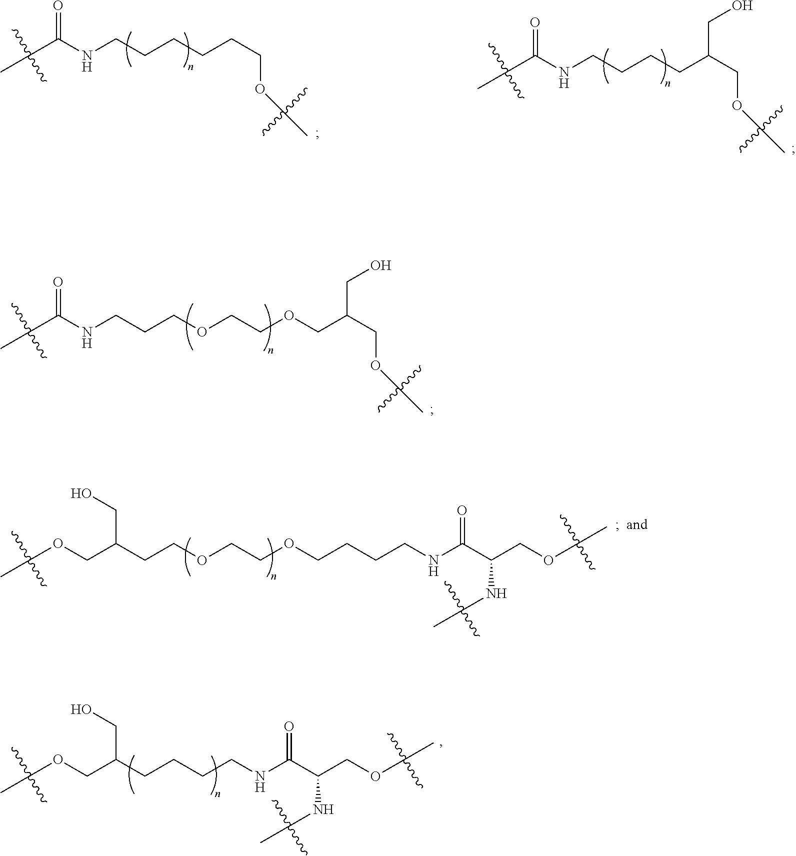
wherein n is 1, 2, 3, 4, or 5.

58-61. (canceled)

62. A pharmaceutical composition for inhibiting the expression of a S6K1 gene in an organism, comprising the siRNA of claim 1 and a pharmaceutically acceptable carrier.

63-64. (canceled)

65. A method for inhibiting expression of a S6K1 gene in a cell, the method comprising:
(a) introducing into the cell the siRNA of claim 1; and
(b) maintaining the cell produced in step (a) for a time sufficient to obtain degradation of the mRNA transcript of the S6K1 gene, thereby inhibiting expression of the S6K1 gene in the cell.

66. A method of treating or managing an eye disorder comprising administering to a patient in need of such treatment a therapeutically effective amount of the siRNA of claim 1.

67. The method of claim 66, wherein:
the siRNA is administered to the eye of the patient or by intravitreal injection; and/or
the siRNA inhibits the expression of a S6K1 gene by at least 20% or at least 50%.

68-70. (canceled)

71. A vector comprising a regulatory sequence operably linked to a nucleotide sequence that encodes an siRNA substantially complementary to a S6K1 nucleic acid sequence of SEQ ID NOs: 1-6.

72-74. (canceled)

75. A cell comprising the vector of claim 71.

76. A recombinant adeno-associated virus (rAAV) comprising the vector of claim 71 and an AAV capsid.

77. A branched RNA compound comprising two or more of the siRNA of claim 1 covalently bound to one another.

78. (canceled)


SEQUENCE LISTING (84 sequences)

SEQ ID NO: 1 - RNA, 45 nt, synthetic construct
  source 1..45: mol_type = other RNA
aatactgggaagatatttgccatgaaggtgcttaaaaaggcaatg

SEQ ID NO: 2 - RNA, 45 nt, synthetic construct
  source 1..45: mol_type = other RNA
gttggaccatatgaacttggcatggaacattgtgagaaatttgaa

SEQ ID NO: 3 - RNA, 45 nt, synthetic construct
  source 1..45: mol_type = other RNA
ttgcctgtaatacttgcaactaaggacaaattagcatgcaagctt

SEQ ID NO: 4 - RNA, 45 nt, synthetic construct
  source 1..45: mol_type = other RNA
attgatgttttacgtgcaaacaacctgaatcttttttttatataa

SEQ ID NO: 5 - RNA, 45 nt, synthetic construct
  source 1..45: mol_type = other RNA
atctgtacttgaaagtgtgaaagaaaagttttcatttgaaccaaa

SEQ ID NO: 6 - RNA, 45 nt, synthetic construct
  source 1..45: mol_type = other RNA
ccgttgaaatctgatgatgtcaaataagggttatcctaataggca

SEQ ID NO: 7 - RNA, 20 nt, synthetic construct
  source 1..20: mol_type = other RNA
tttgccatgaaggtgcttaa

SEQ ID NO: 8 - RNA, 20 nt, synthetic construct
  source 1..20: mol_type = other RNA
cttggcatggaacattgtga

SEQ ID NO: 9 - RNA, 20 nt, synthetic construct
  source 1..20: mol_type = other RNA
gcaactaaggacaaattagc

SEQ ID NO: 10 - RNA, 20 nt, synthetic construct
  source 1..20: mol_type = other RNA
gcaaacaacctgaatctttt

SEQ ID NO: 11 - RNA, 20 nt, synthetic construct
  source 1..20: mol_type = other RNA
tgtgaaagaaaagttttcat

SEQ ID NO: 12 - RNA, 20 nt, synthetic construct
  source 1..20: mol_type = other RNA
gatgtcaaataagggttatc

SEQ ID NO: 13 - RNA, 45 nt, synthetic construct
  source 1..45: mol_type = other RNA
ttattttctttaaaatcagctattacaggatatttttttattttc

SEQ ID NO: 14 - RNA, 45 nt, synthetic construct
  source 1..45: mol_type = other RNA
ttcctaggttacaagggctagatctaagattattctcatgagaaa

SEQ ID NO: 15 - RNA, 45 nt, synthetic construct
  source 1..45: mol_type = other RNA
ttttcaagtacgaaaagtaacaggagcaaatactgggaagatatt

SEQ ID NO: 16 - RNA, 45 nt, synthetic construct
  source 1..45: mol_type = other RNA
taaggtttgtagtgttacagaataactaaactgggatttataaac

SEQ ID NO: 17 - RNA, 45 nt, synthetic construct
  source 1..45: mol_type = other RNA
tccctttaagcctctgttgcaatctgaagaggatgtgagtcagtt

SEQ ID NO: 18 - RNA, 45 nt, synthetic construct
  source 1..45: mol_type = other RNA
gactgtagcagcattttgagaacttcataattgtagcagtaaatt

SEQ ID NO: 19 - RNA, 45 nt, synthetic construct
  source 1..45: mol_type = other RNA
ataaacaaaatgtatcttagcattaatatcttgagccttgaacat

SEQ ID NO: 20 - RNA, 45 nt, synthetic construct
  source 1..45: mol_type = other RNA
gggtttaatatctgcagagctttataaaatatactgcagtgcata

SEQ ID NO: 21 - RNA, 45 nt, synthetic construct
  source 1..45: mol_type = other RNA
tattacaggatatttttttattttctacattctgttttttaatta

SEQ ID NO: 22 - RNA, 45 nt, synthetic construct
  source 1..45: mol_type = other RNA
ttctaagagaggtgttcatgcttgtaccaggtaagtgaataaaaa

SEQ ID NO: 23 - RNA, 45 nt, synthetic construct
  source 1..45: mol_type = other RNA
tactgttatgctgtttaccttccttaacaattttcttttttgaga

SEQ ID NO: 24 - RNA, 45 nt, synthetic construct
  source 1..45: mol_type = other RNA
cgtaactgcaagccttgggacaggcagaagttgtatgatctacat

SEQ ID NO: 25 - RNA, 45 nt, synthetic construct
  source 1..45: mol_type = other RNA
tttatttgtaaaaactgaagcataatttaaagtgtatatcaatac

SEQ ID NO: 26 - RNA, 45 nt, synthetic construct
  source 1..45: mol_type = other RNA
aattccaataaaacttttacactaaatgattcctccttagcccta

SEQ ID NO: 27 - RNA, 45 nt, synthetic construct
  source 1..45: mol_type = other RNA
aaacccacaaaaaactcaagcaaaatagtattgtggaatccacag

SEQ ID NO: 28 - RNA, 20 nt, synthetic construct
  source 1..20: mol_type = other RNA
tcagctattacaggatattt

SEQ ID NO: 29 - RNA, 20 nt, synthetic construct
  source 1..20: mol_type = other RNA
ggctagatctaagattattc

SEQ ID NO: 30 - RNA, 20 nt, synthetic construct
  source 1..20: mol_type = other RNA
agtaacaggagcaaatactg

SEQ ID NO: 31 - RNA, 20 nt, synthetic construct
  source 1..20: mol_type = other RNA
tacagaataactaaactggg

SEQ ID NO: 32 - RNA, 20 nt, synthetic construct
  source 1..20: mol_type = other RNA
gttgcaatctgaagaggatg

SEQ ID NO: 33 - RNA, 20 nt, synthetic construct
  source 1..20: mol_type = other RNA
ttgagaacttcataattgta

SEQ ID NO: 34 - RNA, 20 nt, synthetic construct
  source 1..20: mol_type = other RNA
cttagcattaatatcttgag

SEQ ID NO: 35 - RNA, 20 nt, synthetic construct
  source 1..20: mol_type = other RNA
agagctttataaaatatact

SEQ ID NO: 36 - RNA, 20 nt, synthetic construct
  source 1..20: mol_type = other RNA
ttttattttctacattctgt

SEQ ID NO: 37 - RNA, 20 nt, synthetic construct
  source 1..20: mol_type = other RNA
tcatgcttgtaccaggtaag

SEQ ID NO: 38 - RNA, 20 nt, synthetic construct
  source 1..20: mol_type = other RNA
taccttccttaacaattttc

SEQ ID NO: 39 - RNA, 20 nt, synthetic construct
  source 1..20: mol_type = other RNA
tgggacaggcagaagttgta

SEQ ID NO: 40 - RNA, 20 nt, synthetic construct
  source 1..20: mol_type = other RNA
tgaagcataatttaaagtgt

SEQ ID NO: 41 - RNA, 20 nt, synthetic construct
  source 1..20: mol_type = other RNA
tttacactaaatgattcctc

SEQ ID NO: 42 - RNA, 20 nt, synthetic construct
  source 1..20: mol_type = other RNA
tcaagcaaaatagtattgtg

SEQ ID NO: 43 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
tattacaggatatta

SEQ ID NO: 44 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
catgaaggtgcttaa

SEQ ID NO: 45 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
catggaacattgtga

SEQ ID NO: 46 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2- Prime-fluoro nucleotides
gatctaagattatta

SEQ ID NO: 47 - RNA, 15 nt, synthetic construct
  source 1..15: mol_type = other RNA
  modified_base order(1..13,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
caggagcaaatacta

SEQ ID NO: 48 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
aataactaaactgga

SEQ ID NO: 49 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
taaggacaaattaga

SEQ ID NO: 50 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
aatctgaagaggata

SEQ ID NO: 51 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
caacctgaatcttta

SEQ ID NO: 52 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
aagaaaagttttcaa

SEQ ID NO: 53 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
aacttcataattgta

SEQ ID NO: 54 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
cattaatatcttgaa

SEQ ID NO: 55 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
tttataaaatataca

SEQ ID NO: 56 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
ttttctacattctga

SEQ ID NO: 57 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
cttgtaccaggtaaa

SEQ ID NO: 58 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
tccttaacaatttta

SEQ ID NO: 59 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
caggcagaagttgta

SEQ ID NO: 60 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
cataatttaaagtga

SEQ ID NO: 61 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
caaataagggttata

SEQ ID NO: 62 - RNA, 15 nt, synthetic construct
  source 1..15: mol_type = other RNA
  modified_base order(1..3,13..15): mod_base = OTHER; note = Phosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
actaaatgattccta

SEQ ID NO: 63 - RNA, 15 nt, synthetic construct
  source 1..15: mol_type = other RNA
  modified_base order(1..3,13..15): mod_base = OTHER; note = hosphorothioate internucleotide linkage
  modified_base order(1..4,8,10..15): mod_base = OTHER; note = 2-Prime-O-methyl nucleotides
  modified_base order(5..7,9): mod_base = OTHER; note = 2-Prime-fluoro nucleotides
caaaatagtattgta

SEQ ID NO: 64 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
taatatcctgtaatagctga

SEQ ID NO: 65 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
ttaagcaccttcatggcaaa

SEQ ID NO: 66 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tcacaatgttccatgccaag

SEQ ID NO: 67 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
taataatcttagatctagcc

SEQ ID NO: 68 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = Prime-O-methyl nucleotides
  modified_base order(2,6,14,16,20): mod_base = OTHER; note = 2-Prime-fluoro nucleotides
tagtatttgctcctgttact

SEQ ID NO: 69 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tccagtttagttattctgta

SEQ ID NO: 70 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tctaatttgtccttagttgc

SEQ ID NO: 71 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tatcctcttcagattgcaac

SEQ ID NO: 72 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = Phosphorothioate internucleotide linkage
  modified_base order(2,6,14,16,20): mod_base = OTHER; note = 2-Prime-fluoro nucleotides
taaagattcaggttgtttgc

SEQ ID NO: 73 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
ttgaaaacttttctttcaca

SEQ ID NO: 74 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tacaattatgaagttctcaa

SEQ ID NO: 75 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
ttcaagatattaatgctaag

SEQ ID NO: 76 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tgtatattttataaagctct

SEQ ID NO: 77 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tcagaatgtagaaaataaaa

SEQ ID NO: 78 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tttacctggtacaagcatga

SEQ ID NO: 79 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
taaaattgttaaggaaggta

SEQ ID NO: 80 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tacaacttctgcctgtccca

SEQ ID NO: 81 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tcactttaaattatgcttca

SEQ ID NO: 82 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tataacccttatttgacatc

SEQ ID NO: 83 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
taggaatcatttagtgtaaa

SEQ ID NO: 84 - RNA, 20 nt, synthetic construct
  source 1..20: mol_type = other RNA
  modified_base order(1..3,13..20): mod_base = OTHER; note = Phosphorothioate internucleotide linkage
  modified_base order(1,3..5,7..13,15,17..19): mod_base = OTHER; note = 2-Prime-O-methyl nucleotides
  modified_base order(2,6,14,16,20): mod_base = OTHER; note = 2-Prime-fluoro nucleotides
tacaatactattttgcttga